(19) United States
(12) Patent Application Publication
(10) Pub. No.: US 2026/0364039 A1
(43) Pub. Date: Nov. 19, 2026

(54) Ophthalmic Compositions to Reduce Preservative-Induced Ocular Surface Damage and Methods of Use Thereof

(71) Applicant: Augusta University Research Institute, Inc., Augusta, GA (US)

(72) Inventors: Shruti Sharma, Martinez, GA (US); Ashok Sharma, Martinez, GA (US); Richard Kontoh-Twumasi, Augusta, GA (US); Amy Estes, Augusta, GA (US)

(21) Appl. No.: 19/664,670

(22) Filed: Apr. 30, 2026

Related U.S. Application Data
(60) Provisional application No. 63/797,739, filed on Apr. 30, 2025.

(51) Int. Cl. A61K 31/205; A61K 31/221; A61P 27/04

(52) U.S. Cl. CPC A61K 31/205; A61K 31/221; A61P 27/04

(57) ABSTRACT

Disclosed are ophthalmic compositions for reducing or preventing ocular surface damage and hyperosmolarity associated with preservatives. The compositions include (i) one or more preservatives, such as benzalkonium chloride (BAC), in an amount effective to reduce microbial contamination, and (ii) one or more osmoprotective agents, such as L-carnitine or a derivative thereof (e.g., acetylcarnitine, propionylcarnitine, butyrylcarnitine, or valerylcarnitine), in an amount effective to mitigate preservative-induced damage. The osmoprotective agent is present in an amount effective to reduce markers of toxicity such as epithelial cell apoptosis, disruption of adherens junctions, tear film instability, and inflammation. The compositions can be formulated as artificial tears, eye drops, contact lens solutions, and ocular wound dressings for topical ocular administration. Methods of using the ophthalmic compositions for reducing preservative-induced ocular surface damage and hyperosmolarity associated with treatments for ocular conditions such as dry eye disease, allergic conjunctivitis, and contact lens discomfort, are also provided.

### CROSS REFERENCE TO RELATED APPLICATIONS

This application claims the benefit of priority to U.S. Provisional Application No. 63/797,739, filed Apr. 30, 2025, the contents of which are specifically incorporated herein by reference in their entirety.

### STATEMENT REGARDING FEDERALLY SPONSORED RESEARCH

This invention was made with government support under grant EY036114 awarded by the National Institutes of Health. The government has certain rights in the invention.

### SEQUENCE LISTING

This application contains a sequence listing filed in ST.26 format entitled “AURI_2025-014_US_ST26.xml” created on Apr. 28, 2026 and having 16,972 bytes. The content of the sequence listing is incorporated herein in its entirety.

### FIELD OF THE INVENTION

The disclosed invention is generally in the field of ophthalmic formulations and specifically in the area of formulations to reduce preservative-induced ocular surface damage and methods of use thereof.

### BACKGROUND OF THE INVENTION

Ophthalmic formulations, including artificial tears and topical medications for conditions such as dry eye disease (DED), glaucoma, and allergic conjunctivitis, frequently include benzalkonium chloride (BAC) as a preservative. BAC is a quaternary ammonium compound widely used for its potent antimicrobial properties, which help prevent microbial contamination in multi-dose containers. Despite its efficacy, BAC has been shown to exacerbate ocular surface toxicity, particularly with prolonged or repeated use.

BAC exerts cytotoxic effects on the corneal and conjunctival epithelium, leading to increased epithelial permeability, apoptosis, inflammation, disruption of tight junctions, and loss of goblet cells. Clinically, these effects contribute to symptoms such as tear film instability, ocular irritation, increased corneal staining, and epithelial erosions. Patients with pre-existing ocular surface disorders such as dry eye disease are particularly vulnerable to these adverse effects. Chronic use of BAC-containing eye drops, which is common in the treatment of eye diseases such as glaucoma, can significantly worsen ocular surface health, reduce patient compliance, and impair visual outcomes.

Despite widespread recognition of benzalkonium chloride's ocular toxicity, current strategies to improve preservative tolerability remain suboptimal. For example, preservative-free formulations have been introduced to improve tolerability; however, they present challenges related to microbial control, increased manufacturing costs, reduced shelf life, and limited feasibility in multi-dose packaging. Similarly, alternative preservatives such as Polyquad®, Purite®, and SofZia® offer only modest improvements in ocular tolerability and still elicit inflammatory or cytotoxic responses, particularly in sensitive or compromised patients.

There remains an urgent need for improved ophthalmic formulations that retain the antimicrobial efficacy of BAC; reduces or eliminates its cytotoxic effects on the ocular surface; improves epithelial barrier function and tear film stability; and is compatible with long-term, repeated use, for example in vulnerable patient populations.

Therefore, it is an object of the invention to provide improved ophthalmic compositions that retain the antimicrobial efficacy of preservatives e.g., BAC, while minimizing their cytotoxic effects.

It is also an object of the invention to provide improved ophthalmic formulations suitable for long-term use and that improve epithelial barrier integrity, reduce inflammation, and improve tear film stability.

It is another object of the invention to provide methods of treating or preventing preservative-induced ocular surface damage.

### SUMMARY OF THE INVENTION

Ophthalmic compositions for reducing or preventing hyperosmolarity and/or ocular surface damage associated with preservatives (e.g., BAC) that induce hyperosmolar stress are provided. In some forms, the preservative causes or increases dry eye. The ophthalmic composition contains: (i) one or more preservatives, in an amount effective to prevent or reduce microbial contamination, and (ii) one or more osmoprotective agents, in an amount effective to reduce preservative-induced hyperosmolarity and/or ocular surface damage. In some forms, the preservative is benzalkonium chloride (BAC). In some forms, the one or more osmoprotective agents are L-carnitine and/or an L-carnitine derivative/analog (such as acetylcarnitine, propionylcarnitine, butyrylcarnitine, or valerylcarnitine). The reduction and/or prevention of preservative-induced hyperosmolarity and/or ocular surface damage of the disclosed formulations can be in comparison to the same formulation without the osmoprotective agent.

The preservative (such as BAC) is typically present in the ophthalmic composition in a concentration ranging from about 0.001% wt/v to about 0.02% wt/v and the osmoprotective agent (such as L-carnitine or a derivative/analog thereof) is typically present in the ophthalmic composition in a concentration ranging from about 0.1% to about 5.0% wt/v.

The compositions can be formulated as aqueous solutions, suspensions, gels, ointments, or emulsions suitable for topical ocular administration, and can optionally include one or more pharmaceutically acceptable carriers, one or more viscosity modifiers, one or more lubricants, one or more buffering agents, and/or one or more additional active ingredients such as anti-inflammatory drugs, anti-glaucoma agents, antibiotics, or antihistamines. In some forms, the compositions can be formulated into various ocular forms including but not limited to eye drops, artificial tears, contact lens solutions, ocular inserts, or ocular wound dressings. Formulations containing the ophthalmic compositions generally have an osmolality ranging from 150 mOsm/kg to 500 mOsm/kg, optionally from 280 mOsm/kg to 320 mOsm/kg.

Methods of using the ophthalmic compositions are also provided. The disclosed methods include administering to the eye of a subject in need thereof, an effective amount of the ophthalmic compositions described above, to reduce and/or prevent ocular surface hyperosmolarity and related damage. In some forms of the disclosed methods, the preservative and the osmoprotective agent are administered in separate compositions, e.g., concurrently or sequentially. The methods are effective in reducing hyperosmolarity that is induced by preservatives (e.g., benzalkonium chloride) and optionally other ingredients in ophthalmic formulations used to treat ocular conditions. The reduction and/or prevention of preservative-induced hyperosmolarity and/or ocular surface damage can be compared to a subject being administered the same formulation without the osmoprotective agent. Exemplary conditions for which the ophthalmic compositions can be used include, but are not limited to, dry eye disease, allergic conjunctivitis, blepharitis, corneal defects, contact lens discomfort, post-operative inflammation, and ocular surface disease associated with autoimmune disorders. In some forms, the compositions are not used to treat the primary condition itself, but are instead used to prevent or reduce hyperosmolarity-induced ocular surface damage, that occurs secondary to the treatment or management of these conditions.

Additional advantages of the disclosed method and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings illustrate several embodiments of the disclosed method and compositions and together with the description, serve to explain the principles of the disclosed method and compositions.

FIG. 1 is a line graph showing hyperosmolarity induces a dose-dependent decline in corneal epithelial barrier function. Telomerase-immortalized human corneal epithelial cells were (hTCEpi) treated with 70, 90, and 120 mM NaCl (corresponding to 450, 500, and 550 mOsM, respectively) for 24 hours exhibited a dose-dependent reduction in trans-epithelial electrical resistance (TER). The arrow indicates the timepoint when NaCl treatments were added to the cells. Results are presented as mean±SEM, n=16-17 per group, with ****p<0.0001 compared to untreated (UT).

FIGS. 2A-2D are bar graphs showing that hyperosmolar stress decreases E-cadherin levels in corneal epithelial cells. hTCEpi cells treated with 90 mM (FIGS. 2A and 2B) and 120 mM NaCl (FIGS. 2C and 2D) for 24 hours showed a significant reduction in E-cadherin RNA expression and protein levels. Gene expression was normalized to GAPDH, and protein levels were normalized to β-actin. Results are presented as mean±SEM, n=4-8 per group, with *p<0.05, **p<0.01, ****p<0.0001 compared to untreated (UT).

FIGS. 3A-3D are bar graphs showing increased osmolarity leads to decreased β-catenin levels in corneal epithelial cells. hTCEpi cells treated with 90 and 120 mM NaCl for 24 hours exhibited a significant decline in β-catenin protein levels (FIGS. 3B and 3D). No significant changes in β-catenin RNA expression were observed with 90 mM NaCl treatment (FIG. 3A), but significant decrease was observed in 120 mM NaCl group (FIG. 3C). Gene expression was normalized to GAPDH, and protein levels were normalized to β-actin. Results are presented as mean±SEM, n=4-24 per group, with *p<0.05, **p<0.01 compared to untreated (UT); ns=non-significant.

FIGS. 4A-4D are bar graphs showing that increased osmolarity dysregulates α-catenin and p120-catenin expression in corneal epithelial cells. hTCEpi cells treated with 90 and 120 mM NaCl for 24 hours showed a significant decline in p120-catenin gene expression (FIGS. 4A and 4B). α-catenin expression was significantly elevated following 90 mM NaCl treatment but showed no significant change with 120 mM NaCl (FIG. 4C and FIG. 4D). Results are presented as mean±SEM, n=6-30 per group, with **p<0.01, ****p<0.0001 compared to untreated (UT); ns=non-significant.

FIGS. 5A and 5B show that dose-dependent hyperosmolar stress impairs corneal epithelial wound healing. Wound healing was assessed in hTCEpi cells treated with 70, 90, and 120 mM NaCl (450, 500, and 550 mOsM, respectively). FIG. 5A is a line graph depicting normalized trans-epithelial resistance (TER) measured at a frequency of 4000 Hz after wounding with a 5,000 μA current for 30 seconds. FIG. 5B is a table showing results indicate delayed cell migration time and impaired migration rate in a dose-dependent manner. Results are presented as mean±SEM, n=3-4 per group, with ****p<0.0001 compared to untreated (UT); ns=non-significant; W=wounding.

FIGS. 6A-6G show that L-carnitine supplementation partially restores hyperosmolarity-induced epithelial barrier dysfunction and adherens junction protein integrity. FIG. 6A is a line graph that shows hTCEpi cells treated with 90 mM NaCl with and without 200 mM L-carnitine supplementation showed improved epithelial barrier function. FIGS. 6B and 6E are representative immunofluorescence images of E-cadherin (FIG. 6B) and β-catenin (FIG. 6E) in untreated (UT), 90 mM NaCl-treated, and 90 mM NaCl+200 mM L-carnitine (LCAR)-treated cells. Cells exposed to 90 mM NaCl show markedly reduced E-cadherin and β-catenin levels, while co-treatment with LCAR restores these proteins to levels comparable to control. Yellow arrows indicate intercellular adherens junctions. FIGS. 6C and 6F are bar graphs showing quantification of E-cadherin and β-catenin relative fluorescence intensity (RFI) normalized to total cell count (8-10 images/group). FIGS. 6D and 6G are bar graphs showing mRNA expression levels of E-cadherin and β-catenin measured by RT-PCR and shown as fold change. Data are presented as mean±SEM (n=5-10 per group). Statistical significance: *p<0.05, **p<0.01, ***p<0.001, ****p<0.0001. Scale bar=50 μm.

FIGS. 7A-7D are bar graphs show that L-carnitine supplementation restores hyperosmolarity-induced dysregulation of Wnt/β-catenin signaling modulators. FIGS. 7A and 7B are RT-PCR analysis of Wnt pathway components under hyperosmotic stress (90 mM NaCl) with or without L-carnitine (200 mM) supplementation. FIG. 7C and FIG. 7D show expression of Wnt signaling activators WNT1 and LRP6 was significantly decreased in cells treated with 90 mM NaCl but was restored to near control levels following L-carnitine supplementation. Conversely, expression of Wnt pathway inhibitors DKK1 and WIF1 was upregulated under hyperosmolar conditions, and L-carnitine treatment significantly attenuated this effect. Data are presented as mean±SEM (n=7-33 per group). Statistical significance: *p<0.05, **p<0.01, ***p<0.001.

FIG. 8 is a schematic representation of the effects of hyperosmolarity on adherens junction proteins and Wnt/β-catenin signaling. FIG. 8 illustrates how hyperosmolarity affects epithelial barrier integrity mediated through the adherens junction complex and the Wnt/β-catenin signaling pathway under normal and hyperosmolar conditions.

FIG. 9 is a line graph showing that benzalkonium chloride (BAC) impairs epithelial barrier function in a dose-dependent manner. Human corneal epithelial cells were treated with increasing concentrations of BAC (0.002%, 0.02%, and 0.2%), and epithelial barrier function was assessed over time. A decline in barrier integrity was observed following BAC treatment, with greater impairment at higher concentrations. Timepoint zero (0) denotes the initiation of treatment. Data are presented as mean±SEM (n=4 per group). Statistical significance: ****p<0.0001.

FIG. 10A is a bar graph showing treating human corneal epithelial cells (hTCEpi) with 0.002% Benzalkonium chloride (BAC) after 24 hours show decreased expression of adherens junction protein, E-cadherin. Results are expressed as Mean±SEM, n=4/group, ****p-value <0.0001 vs UT(untreated). FIG. 10B is a bar graph showing treatment of human corneal epithelial cells (hTCEpi) with 0.002% Benzalkonium chloride (BAC) after 24 hours show decreased expression of adherens junction protein, β-catenin. Results are expressed as Mean±SEM, n=4/group, **p-value=0.0029 vs UT(untreated).

FIGS. 11A and 11B are bar graphs showing treatment of human corneal epithelial cells (hTCEpi) treated with 0.002% Benzalkonium chloride (BAC) after 6 hours show decreased expression of adherens junction protein, E-cadherin (FIG. 11A) and β-catenin (FIG. 11B). Results are expressed as Mean±SEM, n=4/group, *p-value=0.0487, **p-value=0.0056 vs UT(untreated).

FIG. 12 is a bar graph of tear film measurement with phenol red thread on mice eyes treated with/without 0.2% BAC and 200 mM L-carnitine. One-way ANOVA analysis with Tukey's multiple comparison. Results are expressed as mean±SEM, n=16-24 eyes/group, ****p<0.0001.

FIGS. 13A and 13B are bar graphs of tear film measurement with phenol red thread on male (FIG. 13A) and female (FIG. 13B) mice eyes treated with/without 0.2% BAC and 200 mM L-carnitine. One-way ANOVA analysis with Tukey's multiple comparison. Results are expressed as mean±SEM, n=16-24 eyes/group, ****p<0.0001.

FIGS. 14A and 14B are bar graphs showing scores of corneal opacity (FIG. 14A) and corneal damage (FIG. 14B) of mice treated with/without 0.2% BAC and 200 mM L-carnitine. One-way ANOVA analysis with Tukey's multiple comparison. Results are expressed as mean±SEM, n=16-24 eyes/group, ****p<0.0001.

FIGS. 15A and 15B are bar graphs showing scores of corneal opacity of male (FIG. 15A) and female (FIG. 15B) mice eyes treated with/without 0.2% BAC and 200 mM L-carnitine. One-way ANOVA analysis with Tukey's multiple comparison. Results are expressed as mean±SEM, n=8-12 eyes/group, ****p<0.0001.

FIGS. 16A and 16B are bar graphs showing corneal damage scores of male (FIG. 16A) and female (FIG. 16B) mice eyes treated with/without 0.2% BAC and 200 mM L-carnitine. One-way ANOVA analysis with Tukey's multiple comparison. Results are expressed as mean±SEM, n=8-12 eyes/group, ****p<0.0001.

FIGS. 17A and 17B are bar graphs showing analysis of overall corneal thickness (FIG. 17A) and corneal epithelium thickness (FIG. 17B) following treatment with/without 0.2% BAC and 200 mM L-carnitine in mice eyes. One-way ANOVA analysis with Tukey's multiple comparison. Results are expressed as mean±SEM, n=9-37 sections/group, *p<0.05, **p<0.01, ***p<0.001****p<0.0001, ns=non-significant.

FIGS. 18A and 18B shows L-carnitine mitigates hyperosmolarity-induced increases in free fatty acid uptake and triglyceride levels. Human telomerase-immortalized corneal epithelial (hTCEpi) cells were treated with 90 mM NaCl corresponding to osmolarity of 490 mOsM with or without 200 mM L-carnitine (LCAR) supplementation. FIG. 18A is a line graph showing that Free fatty acid (FFA) uptake was significantly increased following NaCl treatment and reduced after LCAR supplementation, n=15-16/group. FIG. 18B is a bar graph showing that Triglyceride (TG) levels were elevated under hyperosmolar conditions and decreased upon LCAR supplementation, n=11-12/group. Results are presented as mean±SEM. Statistical significance was determined by one-way ANOVA followed by Holm-Šidák's multiple comparisons test (*p<0.05, ***p<0.001).

FIGS. 19A-19C show that hyperosmolar stress-induced lipotoxicity is attenuated by L-carnitine supplementation. Human telomerase-immortalized corneal epithelial (hTCEpi) cells were exposed to 90 mM NaCl corresponding to osmolarity of 490 mOsM with or without 200 mM L-carnitine (LCAR) supplementation. Fluorescence microscopy was performed and intracellular lipid distribution following Nile Red staining was observed. FIG. 19A is a bar graph showing quantification of Nile Red relative fluorescence intensity (RFI) per cell (n=15-20 images/group), normalized to the untreated (UT) control. FIG. 19B is a bar graph showing malondialdehyde (MDA) levels, a marker of lipid peroxidation, were significantly elevated under hyperosmolar conditions and reduced following LCAR treatment, n=10-11/group. FIG. 19C is a bar graph of western blot analysis showing decreased peroxisome proliferator-activated receptor alpha (PPARα) levels under hyperosmolar stress, which was significantly increased following LCAR supplementation,n=3-4/group. Results are presented as mean±SEM. Statistical significance was determined by one-way ANOVA followed by Holm-Šidák's multiple comparisons test (**p<0.01, ****p<0.0001).

FIGS. 20A-20C shows the effects of L-carnitine treatment on mitochondrial oxidative stress and ATP levels. Human telomerase-immortalized corneal epithelial (hTCEpi) cells were exposed to 90 mM NaCl corresponding to osmolarity of 490 mOsM with or without 200 mM L-carnitine (LCAR) supplementation. FIG. 20A is a bar graph showing quantification of MitoSOX Red relative fluorescence intensity (RFI) showing increased mitochondrial reactive oxygen species (mtROS) under hyperosmolar conditions, which were partially reduced with LCAR treatment, n=15-20 images/group. Scale bar: 100 m. FIG. 20B is a bar graph of quantification of western blot analysis of inducible nitric oxide synthase (iNOS) showing significantly elevated protein levels in cells exposed to 90 mM NaCl, which was significantly decreased with LCAR supplementation, n=3-4/group. FIG. 20C is a bar graph showing intracellular ATP levels (nmol) were significantly reduced under hyperosmolar stress and recovered following LCAR treatment, n=4-6/group. Results are presented as mean±SEM. Statistical significance was determined by one-way ANOVA followed by Holm-Šidák's multiple comparisons test (*p<0.05, **p<0.01, ***p<0.001, ****p<0.0001).

FIGS. 21A-21E are bar graphs showing that L-carnitine partially preserves carnitine palmitoyltransferase (CPT) levels and activity under hyperosmolar stress. Human telomerase-immortalized corneal epithelial (hTCEpi) cells were exposed to 90 mM NaCl corresponding to osmolarity of 490 mOsM with or without 200 mM L-carnitine (LCAR) supplementation. FIGS. 21A and 21B show CPT1 mRNA expression (n=8-14/group) and protein levels (n=16-20/group) were significantly decreased under hyperosmolar stress but only RNA levels were restored after LCAR treatment. FIGS. 21C and 21D show CPT2 mRNA expression (n=4-6/group) and protein levels (n=5-9/group) showed similar decreases following NaCl treatment, which were partially recovered with LCAR supplementation. FIG. 21E show total CPT activity (nmol/min/mg protein) showed a significant decrease following hyperosmolar stress that was partially restored by pre-treatment with LCAR (n=5-6/group). Results are presented as mean±SEM. Statistical significance was determined by one-way ANOVA followed by Holm-Šidák's multiple comparisons test (*p<0.05, **p<0.01, ***p<0.001, ****p<0.0001, ns=non-significant).

FIGS. 22A-22C are bar graphs showing that L-carnitine partially restores carnitine acetyltransferase (CrAT) expression and activity under hyperosmolar stress. Human corneal epithelial (hTCEpi) cells were exposed to 90 mM NaCl corresponding to osmolarity of 490 mOsM with or without 200 mM L-carnitine (LCAR) treatment. FIGS. 22A and 22B show CrAT mRNA expression (n=3-4/group) and protein levels (n=7-9/group) were significantly decreased under hyperosmolar conditions and partially restored by LCAR supplementation. FIG. 22C show total CrAT activity (nmol/min/mg protein) showed a significant decrease following hyperosmolar stress that was partially prevented by pre-treatment with LCAR (n=3-7/group). Results are presented as mean±SEM. Statistical significance was determined by one-way ANOVA followed by Holm-Šidák's multiple comparisons test (*p<0.05, **p<0.01, ***p<0.001, ****p<0.0001).

FIG. 23 is a line graph showing hyperosmolarity induces a dose-dependent decline in human corneal epithelial barrier function. Telomerase-immortalized human corneal epithelial cells (hTCEpi) treated with 70, 90, and 120 mM NaCl corresponding to final osmolarities of ~450, 490, and 550 mOsm/L, respectively, for 24 hours exhibited a dose-dependent reduction (30-69%) in trans-epithelial electrical resistance (TER). Timepoint zero (0) indicates when NaCl treatments were added to the cells. Results are presented as mean±SD, n=19-20/group, with ****p<0.0001 compared to untreated (UT).

FIGS. 24A-24D are bar graphs showing that hyperosmolar stress decreases E-cadherin levels in human corneal epithelial cells. hTCEpi cells exposed to media with 490 mOsM (FIGS. 24A, 24B) and 550 mOsM (FIGS. 24C, 24D) for 24 hours showed a significant reduction in E-cadherin RNA expression and protein levels. Gene expression was normalized to GAPDH, and protein levels were normalized to β-actin. Results are presented as mean±SD, n=4-8/group, with *p<0.05, ****p<0.0001 compared to untreated (UT).

FIGS. 25A-25D are bar graphs showing increased osmolarity leads to decreased β-catenin levels in human corneal epithelial cells. hTCEpi cells treated with 90 and 120 mM NaCl corresponding to osmolarities of 490 and 550 mOsM for 24 hours exhibited a significant decline in β-catenin protein levels (FIGS. 25B, 25D). No significant changes in (3-catenin RNA expression were observed with 490 mOsM treatment (FIG. 25A), but significant decrease was observed in 550 mOsM group (FIG. 25C). Gene expression was normalized to GAPDH, and protein levels were normalized to β-actin. Results are presented as mean±SD, n=4-24/group, with *p<0.05, **p<0.01 compared to untreated (UT); ns=non-significant.

FIGS. 26A-26D are bar graphs showing increased osmolarity alters α-catenin and p120-catenin expression in human corneal epithelial cells. hTCEpi cells exposed to media with 490 and 550 mOsM for 24 hours showed a significant decline in p120-catenin gene expression (FIG. 26A, FIG. 26B). α-catenin mRNA levels were significantly elevated following 490 mOsM treatment but showed no significant change with 550 mOsM (FIG. 26C, FIG. 26D). Results are presented as mean±SD, n=6-30/group, with **p<0.01, ****p<0.0001 compared to untreated (UT); ns=non-significant.

FIGS. 27A-27D are bar graphs showing L-carnitine supplementation partially restores hyperosmolarity-induced loss of adherens junction protein integrity. Immunostaining of E-cadherin (red) and β-catenin (green) showed decreased levels in the 490 mOsM group compared to untreated (UT). However, L-carnitine (LCAR) supplementation significantly improved E-cadherin (FIG. 27A) and β-catenin (FIG. 27B) fluorescence intensity and RNA expression (FIG. 27C, FIG. 27D). Arrows indicate intercellular adherens junctions. For immunostaining results, the relative fluorescence intensity (RFI) was measured (8-10 images/group) and normalized to total cell count. RT-PCR results are presented as mean SD, n=5-10/group, with *p<0.05, **p<0.01, ***p<0.001, ****p<0.0001. Scale bar=50 μm.

FIGS. 28A and 28B are line graphs showing hyperosmolarity-induced epithelial barrier dysfunction and wound healing are partially restored after L-carnitine treatment. hTCEpi cells expose to media with 490 mOsM with and without 200 mM L-carnitine (LCAR) supplementation showed partially restored epithelial barrier function (FIG. 28A). Wound healing assay (5,000 μA current for 30 seconds) in hTCEpi cells treated with 490 mOsM demonstrated delayed cell migration rate. However, L-carnitine treatment showed protection against the induced wound (FIG. 28B). Graphs depict normalized trans-epithelial resistance (TER) measured at a frequency of 4000 Hz. Results are presented as mean±SD, n=4-6/group, with ****p<0.0001 compared to untreated (UT); W=wounding.

FIGS. 29A and 29B shows that L-carnitine ameliorates BAC-induced corneal opacity in male and female mice. Slit lamp microscopy was performed of male and female mice eyes exposed to 5 μL of 0.2% benzalkonium chloride (BAC) with or without L-carnitine (LCAR) twice daily for 7 days. Scores of corneal opacity of male (FIG. 29A) and female mice (FIG. 29B) following treatments were evaluated using a system described in the methods. Results are expressed as mean±SEM, n=12-20 eyes/group, ****p<0.0001, ns=non-significant. Vehicle group received topical administration of water.

FIGS. 30A-30D shows that L-carnitine improves BAC-associated corneal damage and tear insufficiency in male and female mice. Slit lamp microscopy and fluorescence sodium staining was performed of male and female mice corneas administered with 0.2% benzalkonium chloride (BAC) with or without L-carnitine (LCAR). Arrows indicate regions of corneal damage. Scoring of corneal damage in male (FIG. 30A) and female mice (FIG. 30B) after various treatments was done using a system described in the methods. Measurement of tear film volume via phenol red thread test in male (FIG. 30C) and female mice (FIG. 30D). Results are expressed as mean±SEM, n=12-20 eyes/group, *p<0.05, **p<0.01, ****p<0.0001, ns=non-significant. Vehicle=water.

FIGS. 31A-31D shows corneal morphology is preserved by L-carnitine in male and female BAC-treated mice. H & E staining was performed of corneal sections from male and female mice following topical administration with 0.2% benzalkonium chloride (BAC) with or without L-carnitine (LCAR). Thickness of the total cornea and the corneal epithelium only were measured in both male (FIG. 31A, FIG. 31B) and female mice (FIG. 31C, FIG. 31D). Images were captured at 2× and 20× magnifications with scale bars corresponding to 200 and 50 μm, respectively. Results are expressed as mean±SEM, n=3-7 mice/group, ***p<0.001, ****p<0.0001, ns=non-significant. Vehicle=water.

FIGS. 32A-32C shows that BAC-impaired corneal epithelial and stromal layers are protected with L-carnitine treatment. Western blot analysis for corneal epithelial marker, E-cadherin (n=6-12/group) and stromal markers, ALDH3A1 (n=4-5/group) and Keratocan (n=4-13/group) (FIG. 32A, FIG. 32B, FIG. 32C), in mice corneal homogenates after topical administration with 0.2% benzalkonium chloride (BAC) with or without L-carnitine (LCAR). Protein levels were normalized to β-actin. Results are expressed as mean±SEM, *p<0.05, **p<0.01, ***p<0.001, ****p<0.0001, ns=non-significant. Vehicle=water.

FIGS. 33A-33C shows that L-carnitine restores BAC-induced dysregulation of carnitine metabolism enzymes. Immunostaining was performed of CPT1, CPT2 and CRAT enzymes in mice corneal sections after topical treatment with 0.2% benzalkonium chloride (BAC) with or without L-carnitine (LCAR). The corresponding fluorescence intensities of the enzymes were quantified: CPT1 (FIG. 33A), CPT2 (FIG. 33B), CrAT (FIG. 33C). Nuclei were stained with DAPI (Blue). Images were taken at 20× magnification and scale bar=50 μm. Results are expressed as mean±SEM, n=3-6 mice/group, *p<0.05, **p<0.01,***p<0.001, ns=non-significant. Vehicle=water.

FIG. 34 shows that BAC-associated apoptosis in mice corneas is rescued with L-carnitine administration. TUNEL staining was performed for dead cells in mice corneal sections following administration with 0.2% benzalkonium chloride (BAC) with or without L-carnitine (LCAR). Nuclei were stained with DAPI (Blue). The graph represents the average number of TUNEL-positive cells. Results are expressed as mean±SEM, n=3 mice/group, **p<0.01, ns=non-significant. Vehicle=water.

### DETAILED DESCRIPTION OF THE INVENTION

The disclosed method and compositions can be understood more readily by reference to the following detailed description of particular embodiments and the Example included therein and to the Figures and their previous and following description.

### I. DEFINITIONS

“Active Agent,” as used herein, refers to a physiologically or pharmacologically active substance that acts locally and/or systemically in the body. An active agent is a substance that is administered to a patient for the treatment (e.g., therapeutic agent), prevention (e.g., prophylactic agent), or diagnosis (e.g., diagnostic agent) of a disease or disorder. “Ophthalmic Drug” or “Ophthalmic Active Agent”, as used herein, refers to an agent that is administered to a patient to alleviate, delay onset of, or prevent one or more symptoms of a disease or disorder of the eye, or diagnostic agent useful for imaging or otherwise assessing the eye.

As used herein, “hyperosmolarity” refers to a condition in which the osmotic concentration of a fluid, such as the tear film, exceeds normal physiological levels, resulting in an increased concentration of solutes relative to the intracellular environment.

The term, “in vitro” refers to an artificial environment and to processes or reactions that occur within an artificial environment. In vitro environments include, but are not limited to, in solution or suspension, and cell cultures.

The term “in vivo” refers to in or associated with an organism, such as an animal.

As used herein, the terms “subject,” “individual,” and “patient” refer to any individual who is the target of treatment using the disclosed formulations. The subject can be a vertebrate, for example, a mammal. Thus, the subject can be a human. The subjects can be symptomatic or asymptomatic. The term does not denote a particular age or sex. Thus, adult, aged adult, and newborn subjects, whether male or female, are intended to be covered. A subject can include a control subject or a test subject.

As used herein, “treat” means to prevent, reduce, decrease, or ameliorate one or more symptoms, characteristics or comorbidities of an ocular disease, disorder or condition; to reverse the progression of one or more symptoms, characteristics or comorbidities of an ocular disorder; to halt the progression of one or more symptoms, characteristics or comorbidities of an ocular disorder; to prevent the occurrence of one or more symptoms, characteristics or comorbidities of an ocular disorder; to inhibit the rate of development of one or more symptoms, characteristics or comorbidities or combinations thereof.

As used herein, the term “effective amount” or “therapeutically effective amount” means a dosage sufficient to treat, inhibit, or alleviate one or more symptoms of a disease state being treated or to otherwise provide a desired pharmacologic and/or physiologic effect. The effective amount required will vary from subject to subject, depending on subject-dependent variables (e.g., physical conditions, age, sex, immune system health, species, and weight of the subject), the disease state, and the treatment being effected.

As used herein, the term “carrier” or “excipient” refers to an organic or inorganic ingredient, natural or synthetic inactive ingredient in a formulation, with which one or more active ingredients are combined.

As used herein, the term “pharmaceutically acceptable” means a non-toxic material that does not interfere with the effectiveness of the biological activity of the active ingredients. By “pharmaceutically acceptable” is meant a material that is not biologically or otherwise undesirable, i.e., the material can be administered to a subject along with the selected agent without causing any undesirable biological effects or interacting in a deleterious manner with any of the other components of the pharmaceutical composition in which it is contained.

As used herein, the term “pharmaceutically-acceptable carrier” means one or more compatible solid or liquid fillers, diluents or encapsulating substances which are suitable for administration to a human or other vertebrate animal.

The use of the terms “a,” “an,” “the,” and similar referents in the context of the disclosure including the claims are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values may range in value either above or below the stated value in a range of approx. +/−5%; in other forms the values may range in value either above or below the stated value in a range of approx. +/−2%; in other forms the values may range in value either above or below the stated value in a range of approx. +/−1%. The preceding ranges are intended to be made clear by context, and no further limitation is implied.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification the word “comprise,” or variations such as “comprises” or “comprising,” will be understood to imply the inclusion of a stated element, integer or step, or group of elements, integers or steps, but not the exclusion of any other element, integer or step, or group of elements, integers or steps.

### II. Ophthalmic Compositions

Benzalkonium chloride (BAC), a preservative commonly used in topical ophthalmic products such as anti-glaucoma eyedrops, is associated with increased ocular surface toxicity, especially with long-term use. For example, BAC has been shown to disrupt epithelial integrity by compromising tight junctions, increasing epithelial permeability, and inducing apoptosis of corneal and conjunctival epithelial cells (Goldstein, et al., Nature Eye, 36, 361-368 (2022)). BAC is also associated with elevated oxidative stress and exacerbated dry eye symptoms, contributing to reduced ocular surface health and patient discomfort, particularly with long-term use of BAC-containing products (Goldstein, et al., Nature Eye, 36, 361-368 (2022)). Although preservative-free and alternative formulations exist, they generally demonstrate one or more of suboptimal preservative efficacy, issues with regulatory acceptance, potential long-term safety concerns, insufficient protection against microbial contamination, and/or still cause irritation in sensitive patients.

To address these challenges, improved ophthalmic compositions and formulations thereof are provided. The disclosed ophthalmic compositions are based on the findings that maintaining osmotic balance at the ocular surface mitigates the cytotoxic effects of preservatives such as benzalkonium chloride (BAC), without compromising antimicrobial efficacy. As demonstrated in the non-limiting Examples, administration of a formulation containing BAC and L-carnitine reduces corneal opacity, preserves tear film volume, and restores junction protein expression in the corneal epithelium of mouse models of BAC-induced ocular surface toxicity (see Example 2, FIGS. 12, 13A, 13B, 17A and 17B).

Therefore, disclosed are ophthalmic compositions that retain the antimicrobial efficacy of benzalkonium chloride (BAC) and/or other preservative(s) while reducing its associated cytotoxic effects. The ophthalmic compositions generally contain a preservative and an osmoprotective agent such as L-carnitine or a derivative/analog thereof. The preservative is present in the ophthalmic composition in an amount effective to prevent or reduce microbial contamination of the ophthalmic composition. The osmoprotective agent (e.g., L-carnitine or a derivative/analog thereof) is included in an amount effective to reduce or prevent the onset and/or progression of preservative-induced ocular damage. The ophthalmic compositions containing the preservative and the osmoprotective agent can be formulated in a pharmaceutically acceptable carrier suitable for ocular administration.

The disclosed ophthalmic compositions are suitable for use as topical ocular formulations such as artificial tears and anti-glaucoma medications. Additionally, the ophthalmic compositions provide a more tolerable alternative to existing preservative-containing formulations, such as for individuals and patients requiring long-term use or treatment with these formulations.

### A. Osmoprotective Agent

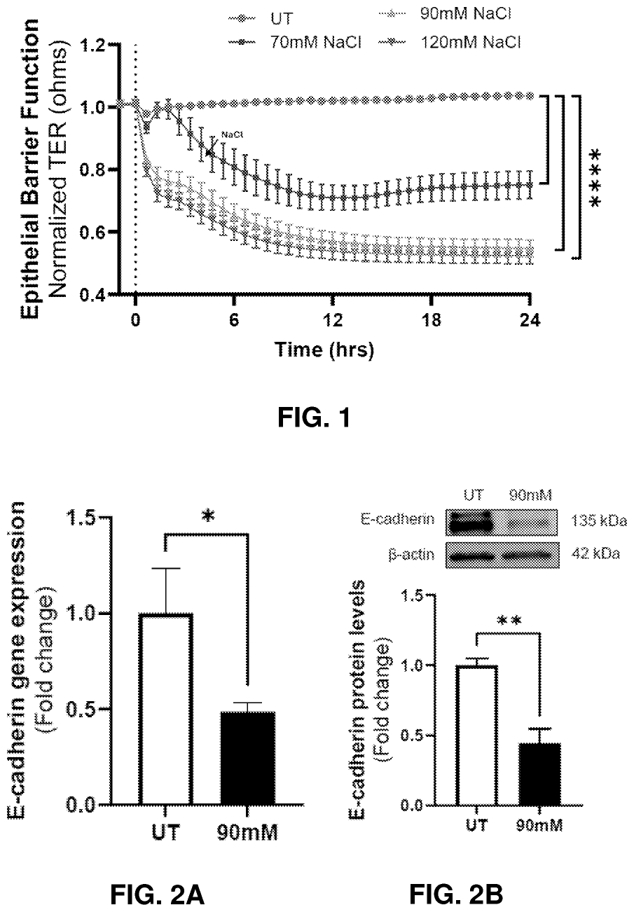

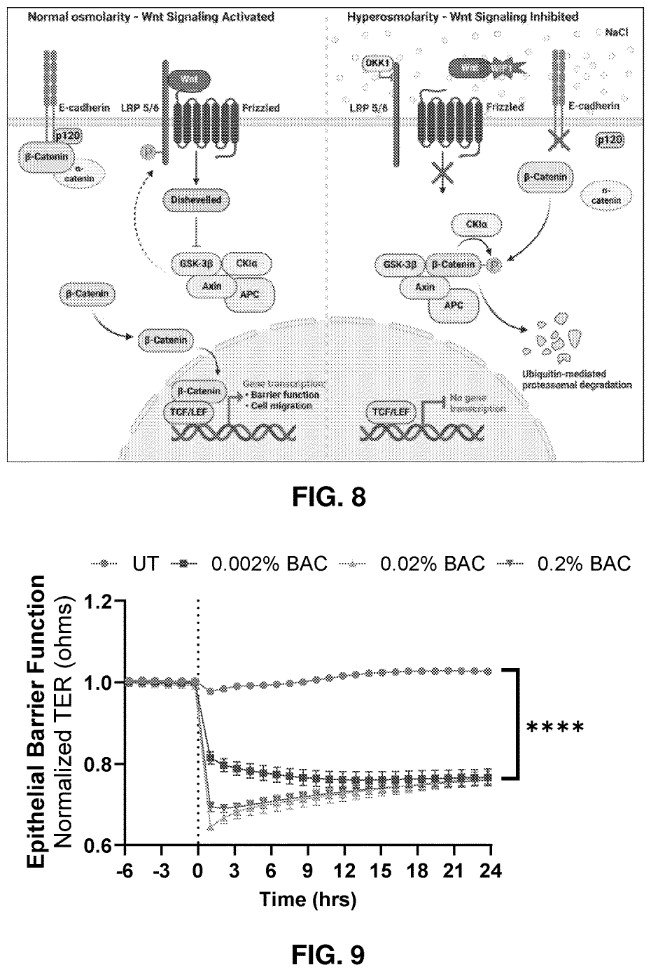

The ocular surface is uniquely vulnerable to environmental and chemical stressors due to its constant exposure and dependence on the protective tear film. The tear film is a thin, multilayered fluid that forms the interface between the external environment and the corneal epithelium, providing lubrication, protection, and optical clarity while supporting ocular surface health through its lipid, aqueous, and mucin components. Osmotic balance between the tear film and the epithelial cells is an important factor in maintaining ocular surface health. When this balance is disrupted, and tear osmolarity rises beyond physiological thresholds, a condition known as hyperosmolarity, substantial damage to the ocular surface epithelium can occur (see FIG. 8). As demonstrated in the non-limiting Examples, hyperosmolar stress induces a dose-dependent decline in corneal epithelial barrier function, decreases β-catenin expression, dysregulates α-catenin and p120-catenin, and impairs epithelial wound healing (see Example 1, FIGS. 1, 2A-2D, 3A-3D, and 4A-4D).

Hyperosmolarity initiates a cascade of biophysical and biochemical changes in corneal and conjunctival epithelial cells. Elevated osmolarity causes immediate water efflux from epithelial cells, resulting in reduced cell volume and increased intracellular concentrations of solutes (International Dry Eye WorkShop. (2007). The Ocular Surface: 2007 Report of the International Dry Eye WorkShop (DEWS). The Ocular Surface, 5(2), 1-204). This physical shrinkage disturbs cytoskeletal organization and cell-cell junctions, weakening epithelial barrier integrity. Tight junction proteins are disrupted, and epithelial permeability increases, rendering the surface more susceptible to further environmental insult, microbial invasion, and inflammation (Dmitrieva, et al., “High NaCl causes Mre11 to leave the nucleus, disrupting DNA damage signaling and repair, Am J Physiol Renal Physiol, 285 (2003), pg. F266-F274). At the molecular level, hyperosmolar conditions induce oxidative stress, mitochondrial dysfunction, and DNA damage (Dmitrieva, et al., “High NaCl causes Mre11 to leave the nucleus, disrupting DNA damage signaling and repair, Am J Physiol Renal Physiol, 285 (2003), pg. F266-F274). These cellular stressors activate apoptosis pathways, including DNA condensation and upregulation of pro-apoptotic signaling (Galvez, et al., “A rapid and strong apoptotic process is triggered by hyperosmotic stress in cultured rat cardiac myocytes”, Cell Tissue Res, 304 (2001), pg. 279-285). If unmitigated, this leads to epithelial cell death, contributing to a loss of goblet cells and disruption of the mucin layer—further destabilizing the tear film and exacerbating epithelial injury. These events impair the glycocalyx and reduce epithelial coverage, creating a feedback loop that perpetuates surface damage and inflammation (reviewed in Baudouin, et al., “Role of hyperosmolarity in pathogenesis and management of Dry Eye Disease: Proceedings of the OCEAN Group Meeting”, The Ocular Surface, 11 (2013), pg. 246-258).

The effects of hyperosmolarity on cellular metabolism are also profound. Protein synthesis is suppressed, macromolecular crowding increases, and osmotic gradients interfere with intracellular transport (Dmitrieva, et al., “High NaCl causes Mre11 to leave the nucleus, disrupting DNA damage signaling and repair, Am J Physiol Renal Physiol, 285 (2003), pg. F266-F274). Regulatory volume increase, the cell's initial compensatory mechanism, involves uptake of electrolytes and water to counteract shrinkage (Burg, et al. “Cellular response to hyperosmotic stresses”, Physiol Rev, 85(2007), pg. 1441-1474). However, prolonged elevation of intracellular inorganic ions disrupts enzyme function and protein structure, making this strategy unsustainable.

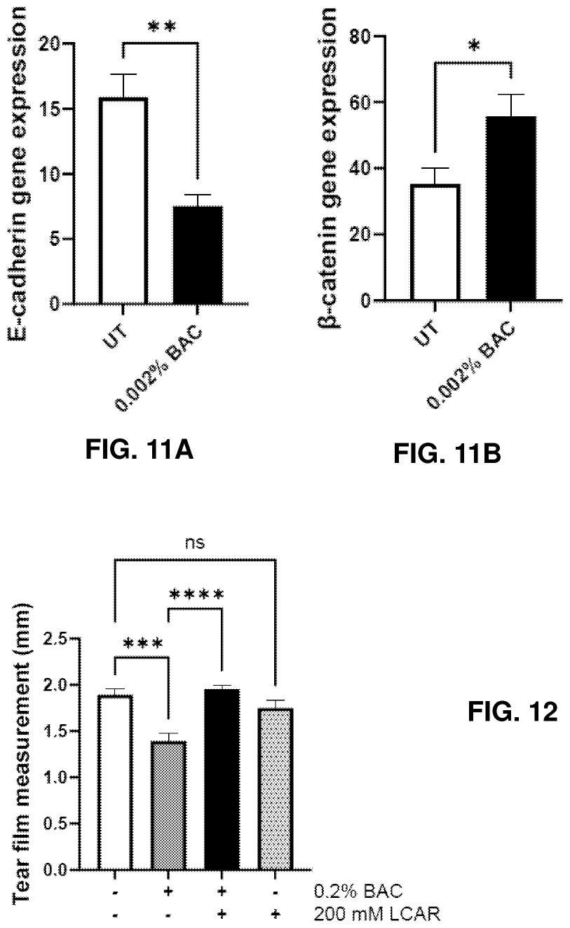

Additionally, BAC induces a concentration-dependent decline in epithelial barrier function, as evidenced by reduced transepithelial electrical resistance (TER) (see non-limiting Example 1, FIG. 9). This decline is consistent with epithelial disruption caused by hyperosmolar stress. BAC exposure also significantly dysregulates the gene expression of adherens junction proteins, including E-cadherin and β-catenin, indicating a rapid and sustained compromise of epithelial integrity (see Non-limiting Example 1, FIGS. 10A and 10B, FIGS. 11A and 11B). These findings demonstrate that BAC contributes to or exacerbates ocular surface hyperosmolarity by destabilizing the epithelial barrier and impairing junctional protein expression, thereby promoting cellular stress responses associated with ocular surface damage.

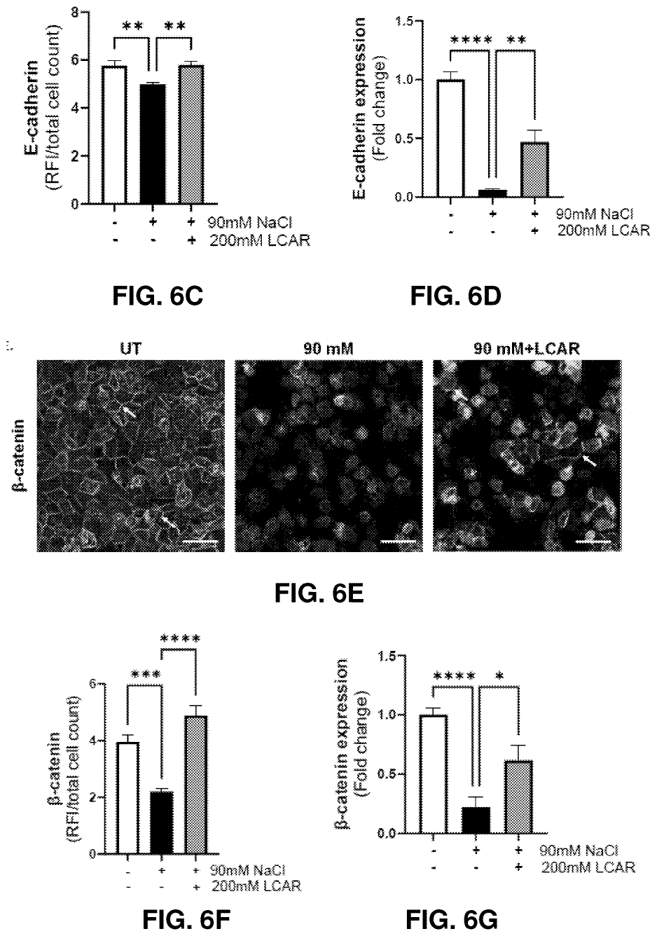

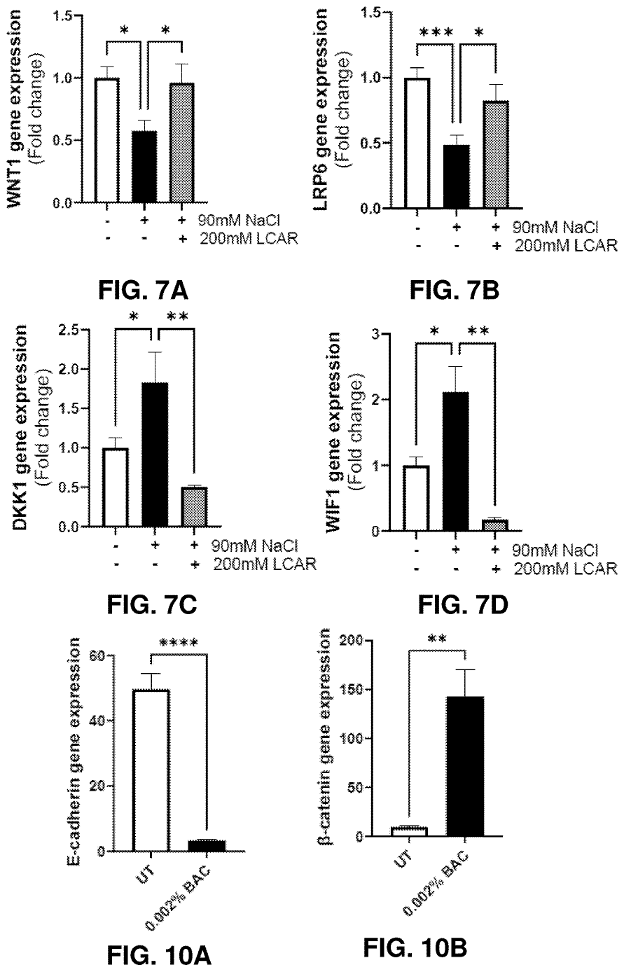

As demonstrated in the non-limiting Examples, administration of an exemplary osmoprotective agent, L-carnitine, significantly restores epithelial barrier integrity and preserves adherens junction protein expression (e.g., E-cadherin and β-catenin) in corneal epithelial cells exposed to hyperosmolar stress in vitro (see Example 1, FIGS. 6A-6G, FIGS. 7A-7D). Additionally, the in vivo studies demonstrate that administration of a formulation containing BAC and L-carnitine reduces corneal opacity, preserves tear film volume, and restores junction protein expression in the corneal epithelium of mouse models of BAC-induced ocular surface toxicity (see Example 2, FIGS. 12, 13A, 13B, 17A and 17B).

Thus, the disclosed ophthalmic compositions combat the effects of hyperosmolarity by including osmoprotective agent in an amount effective to increase osmotic balance and prevent/reduce osmotic stress-induced epithelial injury. In one exemplary form, the amount of the osmoprotective agent in the ophthalmic composition is effective to reduce hyperosmolarity induced by BAC or another preservative, and its associated effects, such as corneal epithelial injury.

The concentration of L-carnitine present in the ophthalmic composition can range from about 0.01% wt/v to about 5.00% weight/volume (wt/v) of the ophthalmic composition.

In some forms, the concentration of L-carnitine present in the ophthalmic composition can range from about 0.05% wt/v to about 4.50% wt/v, from about 0.05% wt/v to about 4.00% wt/v, from about 0.05% wt/v to about 30.50% wt/v, from about 0.05% wt/v to about 3.00% wt/v, from about 0.05% wt/v to about 2.50% wt/v, from about 0.05% wt/v to about 2.00% wt/v, from about 0.05% wt/v to about 1.80% wt/v, from about 0.05% wt/v to about 1.60% wt/v, from about 0.05% wt/v to about 1.40% wt/v, from about 0.05% wt/v to about 1.20% wt/v, from about 0.05% wt/v to about 1.00% wt/v, from about 0.05% wt/v to about 0.80% wt/v, from about 0.05% wt/v to about 0.60% wt/v, from about 0.05% wt/v to about 0.40% wt/v, from about 0.05% wt/v to about 0.20% wt/v, or from about 0.05% wt/v to about 0.08% wt/v, such as about 30.20% wt/v. For example, the L-carnitine is present in the ophthalmic composition in a concentration of at least about 0.05% wt/v, at least about 0.08% wt/v, at least about 0.10% wt/v, at least about 1.20% wt/v, at least about 1.40% wt/v, at least about 1.60% wt/v, at least about 1.80% wt/v, at least about 2.00% wt/v, at least about 30.20% wt/v, up to about 5.00% wt/v.

Generally, the osmoprotective agent in the ophthalmic composition is L-carnitine or a derivative or analog thereof. In some forms, the osmoprotective agent in the ophthalmic composition is L-carnitine. In other forms, the osmoprotective agent is an L-carnitine derivative or analog. Exemplary derivatives/analogs that can be used as the osmoprotective agent are described below.

The osmoprotective agent (e.g., L-carnitine or analog/derivative thereof), can optionally be added to existing or emerging BAC-containing ophthalmic compositions. In some forms, the osmoprotective agent (e.g., L-carnitine or an analog/derivative thereof) can be added to existing preservative-containing (e.g., BAC-containing) ophthalmic formulations with minimal or moderate modification to the base formulation, provided that the ratio of the osmoprotective agent to preservative is sufficient to reduce preservative-induced ocular surface damage (e.g., corneal epithelial disruption) without compromising the antimicrobial efficacy, stability (e.g., shelf life stability), and/or ocular surface permeability of the preservative. Alternatively, adjustments to the base formulation can be made to maintain stability, isotonicity, and pH balance of the ophthalmic composition. Thus, also provided is a methods of making ophthalmic formulations. In some forms, the osmoprotective agent is included at the time of manufacturing the formulation. In some forms, the method of making includes adding an effective amount of osmoprotective agent to a previously prepared eye treatment formulation containing a preservative such as BAC. Such formulations include, but are not limited to, existing commercial eye formulations, treatments, and medicines. Also provided are formulations made in this way.

### 1. L-Carnitine

L-carnitine is a naturally occurring quaternary ammonium compound and plays a role in cellular metabolism. Structurally, L-carnitine is a trimethylated amino acid derivative, chemically classified as p-hydroxy-γ-trimethylaminobutyric acid. Endogenously, L-carnitine is synthesized from the amino acid lysine through a multistep enzymatic pathway. The initial precursor, 6-N-trimethyllysine, is generated via post-translational methylation of lysine residues in proteins and subsequently released through lysosomal proteolysis. This intermediate undergoes a series of enzymatic conversions to form γ-butyrobetaine. The final step of L-carnitine biosynthesis involves the hydroxylation of γ-butyrobetaine to L-carnitine, a reaction catalyzed by γ-butyrobetaine dioxygenase. Endogenous expression of γ-butyrobetaine dioxygenase is restricted to specific tissues, such as the liver, kidney, and brain, which are therefore the primary sites of complete L-carnitine biosynthesis. Other tissues lacking this enzyme rely on the systemic transport of γ-butyrobetaine for conversion to L-carnitine. In biological systems, L-carnitine exists in both its free form and as a variety of acylated derivatives, including short-, medium-, and long-chain acylcarnitines, depending on the nature of the acyl group esterified to the hydroxyl moiety (described further below for acylcarnitines). L-carnitine and its physiological mechanisms are further detailed in Ferreira and McKenna, “L-carnitine and acetyl-L-carnitine roles and neuroprotection in developing brain”, Neurochemical Research, 42(6):1661-1675 (2017), the contents of which are incorporated herein in their entirety. The structure of L-carnitine is provided below.

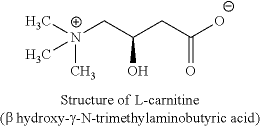

The concentration of L-carnitine present in the ophthalmic composition can range from about 0.01% wt/v to about 5.00% weight/volume (wt/v) of the ophthalmic composition. In some forms, the concentration of L-carnitine present in the ophthalmic composition can range from about 0.05% wt/v to about 4.50% wt/v, from about 0.05% wt/v to about 4.00% wt/v, from about 0.05% wt/v to about 3.50% wt/v, from about 0.05% wt/v to about 3.00% wt/v, from about 0.05% wt/v to about 2.50% wt/v, from about 0.05% wt/v to about 2.00% wt/v, from about 0.05% wt/v to about 1.80% wt/v, from about 0.05% wt/v to about 1.60% wt/v, from about 0.05% wt/v to about 1.40% wt/v, from about 0.05% wt/v to about 1.20% wt/v, from about 0.05% wt/v to about 1.00% wt/v, from about 0.05% wt/v to about 0.80% wt/v, from about 0.05% wt/v to about 0.60% wt/v, from about 0.05% wt/v to about 0.40% wt/v, from about 0.05% wt/v to about 0.20% wt/v, or from about 0.05% wt/v to about 0.08% wt/v, such as about 3.2% wt/v. For example, the L-carnitine is present in the ophthalmic composition in a concentration of at least about 0.05% wt/v, at least about 0.08% wt/v, at least about 0.10% wt/v, at least about 1.20% wt/v, at least about 1.40% wt/v, at least about 1.60% wt/v, at least about 1.80% wt/v, at least about 2.00% wt/v, at least about 3.2% wt/v, up to about 5.00% wt/v.

### 2. Acylcarnitines

Acylcarnitines are esters arising from the conjugation of fatty acids (i.e., acyl groups) with L-carnitine. They are widely used and produced in cellular energy metabolism pathways. One of the biologic functions of acylcarnitines is to transport acyl groups from the cytosol into the mitochondrial matrix for β-oxidation, leading to the production of energy to sustain cell activity. The energy production by β-oxidation is significant, with 7n−6 ATP molecules being produced per acylcarnitine, where n is the number of acyl-carbons.

Most acylcarnitines are classified based on the chemical structure of their variable acyl moiety, resulting in a categorization scheme that parallels the classification of fatty acids—namely into short-, medium-, long-, and very long-chain acylcarnitines. Acylcarnitines may also be grouped according to the saturation level of the fatty acid moiety, including saturated and unsaturated forms. Among the unsaturated acylcarnitines, further subclassification into monounsaturated and polyunsaturated species is common, with consideration given to the cis- or trans-configuration of the double bonds. In addition, acylcarnitines can be categorized by structural features of the acyl chain itself. While the majority contain an aliphatic, straight-chain fatty acid moiety, some acylcarnitines incorporate branched-chain fatty acids or even cyclic organic acid derivatives. Furthermore, the fatty acid moiety may be substituted with functional groups such as hydroxyl or carboxyl groups, contributing to further chemical diversity among acylcarnitine species. Any of the foregoing acylcarnitine is suitable for use as the osmoprotective agent in the ophthalmic composition.

The osmoprotective agent in the ophthalmic composition can be a short chain acylcarnitine, medium chain acylcarnitine, long chain acylcarnitine, or very long chain acylcarnitine. Short-chain acylcarnitine is an ester of L-carnitine and a short-chain fatty acid, wherein the acyl group contains from 2 to 5 carbon atoms. Exemplary short chain acylcarnitines include but are not limited to acetylcarnitine (C2:0), propionylcarnitine (C3:0), butyrylcarnitine (C4:0), and valerylcarnitine (C5:0). As used herein, the notation in brackets (e.g., C2:0, C16:0) refers to the structural characteristics of the acyl group esterified to L-carnitine in an acylcarnitine compound. The “Cx” indicates the number of carbon atoms in the fatty acid chain, while the number following the colon denotes the number of double bonds present in the chain. For example, “C2:0” designates an acyl group with 2 carbon atoms and no double bonds (a saturated short-chain fatty acid), whereas “C16:0” designates an acyl group with 16 carbon atoms and no double bonds (a saturated long-chain fatty acid). A medium-chain acylcarnitine is an ester of L-carnitine and a medium-chain fatty acid, wherein the acyl group contains from 6 to 12 carbon atoms. Exemplary medium chain acylcarnitines include but are not limited to caproylcarnitine (C6:0), octanoylcarnitine (8:0), nonanoylcarnitine (9:0), decanoylcarnitine (C10:0), and lauroylcarnitine (C12:0). A long-chain acylcarnitine is an ester of L-carnitine and a long-chain fatty acid, wherein the acyl group contains from 13 to 20 carbon atoms. Exemplary long chain acylcarnitines include but are not limited to myristoylcarnitine (14:0), palmitoylcarnitine (C16:0), and stearoylcarnitine (C18:0). A very long-chain acylcarnitine is an ester of L-carnitine and a very long-chain fatty acid, wherein the acyl group contains more than 20 carbon atoms. Exemplary very long-chain acylcarnitines include but are not limited to behenoylcarnitine (C22:0), lignoceroylcarnitine (C24:0), and cerotoylcarnitine (C26:0). Any of the foregoing acylcarnitines are suitable for use as the osmoprotective agent in the ophthalmic composition.

The osmoprotective agent can also be an unsaturated chain acylcarnitine. Unsaturated chain acylcarnitines are a large class of acylcarnitines that include acylcarnitines with differing lengths of an unsaturated fatty acid moiety. Exemplary unsaturated chain acylcarnitines include but are not limited to butenylcarnitine (C4:1), hexenoylcarnitine (C6:1), decenoylcarnitine (C10:1), decadienoylcarnitine C10:2), dodecenoylcarnitine (C12:1), tetradecenoylcarnitine (C14:1), tetradecadienoylcarnitine (C14:2), hexadecenoylcarnitine (C16:1), octadecenoylcarnitine (C18:1), and octadecadienylcarnitine. Any of the foregoing unsaturated chain acylcarnitines are suitable for use as the osmoprotective agent in the ophthalmic composition.

In some forms, the osmoprotective agent can be a branched chain acylcarnitine. A branched-chain acylcarnitine is an ester of L-carnitine and a branched-chain fatty acid, typically derived from the catabolism of branched-chain amino acids such as valine, leucine, and isoleucine. The metabolic conversion of these amino acids involves initial deamination to α-keto acids, followed by decarboxylation to form branched-chain acyl-CoA intermediates, which are subsequently conjugated to L-carnitine by mitochondrial or peroxisomal transferases, including carnitine acetyltransferase (CrAT) and carnitine octanoyltransferase (CrOT). Exemplary branched chain acylcarnitines include but are not limited to Isobutyrylcarnitine, Isovalerylcarnitine, 2-Methylbutyrylcarnitine, Methylcrotonylcarnitine, Tiglylcarnitine, Pristanoyl-carnitine, and Phytanoyl-carnitine. Any of the foregoing branched chain acylcarnitines are suitable for use as the osmoprotective agent in the ophthalmic composition.

In other forms, the osmoprotective agent can be a hydroxyl chain acylcarnitine or a dicarboxyl chain acylcarnitine. Exemplary hydroxyl chain and dicarboxyl chain acylcarnitines include but are not limited to 3-Hydroxy-propionylcarnitine (C3-OH), Malonylcarnitine (C3-DC), Methylmalonylcarnitine (C3:0 DC M), 3-Hydroxybutyrylcarnitine (C4-OH), Succinylcarnitine (C4-DC), Glutarylcarnitine (C5-DC), Methylglutarylcarnitine (C5-M-DC), 3-Hydroxyvalerylcarnitine (C5-OH), 3-Hydroxy-isovalerylcarnitine (C5-OH—I), 2-Methyl-3-hydroxy-butyrylcarnitine (C5-OH-M), 3-Methylglutaconylcarnitine (C5:1-M-DC), 3-Hydroxy-3-methylglutaryl-carnitine (C5:1-M-OH-DC), 3-Hydroxy-hexanoylcarnitine (C6-OH), Adipylcarnitine (C6-DC), Dehydroadipylcarnitine (C6:1-DC), Pimelylcarnitine (C7-DC), Suberylcarnitine (C8-DC), Dodecanedioylcarnitine (C12-DC), 3-Hydroxy-tetradecanoylcarnitine (C14-OH), Tetracanedioylcarnitine (C14-DC), 3-Hydroxy-tetradecenoylcarnitine (C14:1—OH), 3-Hydroxy-hexadecanoylcarnitine (C16-OH), 3-Hydroxy-hexadecenoylcarnitine (C16:1—OH), Hexadecanedioylcarnitine (C16-DC), 3-Hydroxysteroylcarnitine (C18-OH), 3-Hydroxyoleylcarnitine (C18:1—OH), and Octadecanedioylcarnitine (C18-DC). Any of the foregoing hydroxyl chain and dicarboxyl chain acylcarnitines are suitable for use as the osmoprotective agent in the ophthalmic composition.

In some forms, the ophthalmic composition contains a single acylcarnitine. Alternatively, the composition may include a combination of two or more different acylcarnitines. For example, the ophthalmic composition may include both a short-chain and a medium-chain acylcarnitine. In other forms, the composition may include combinations such as a short-chain and a long-chain acylcarnitine, a medium-chain and a long-chain acylcarnitine, or acylcarnitines selected from two or more of the following categories: short-chain, medium-chain, long-chain, very long-chain, or branched-chain acylcarnitines.

Additional acylcarnitines, including their synthesis, source, and mechanism of action are further detailed in Dambrova, et al., “Acylcarnitines: Nomenclature, biomarkers, therapeutic potential, drug targets, and clinical trials”, Pharmacological Reviews, 74(3): P506-551 (2022)), the contents of which is incorporated herein by reference in its entirety.

The concentration of the acylcarnitine present in the ophthalmic composition can range from about 0.01% wt/v to about 5.00% weight/volume (wt/v) of the ophthalmic composition.

In some forms, the concentration of the acylcarnitine present in the ophthalmic composition can range from about 0.05% wt/v to about 4.50% wt/v, from about 0.05% wt/v to about 4.00% wt/v, from about 0.05% wt/v to about 3.50% wt/v, from about 0.05% wt/v to about 3.00% wt/v, from about 0.05% wt/v to about 2.50% wt/v, from about 0.05% wt/v to about 2.00% wt/v, from about 0.05% wt/v to about 1.80% wt/v, from about 0.05% wt/v to about 1.60% wt/v, from about 0.05% wt/v to about 1.40% wt/v, from about 0.05% wt/v to about 1.20% wt/v, from about 0.05% wt/v to about 1.00% wt/v, from about 0.05% wt/v to about 0.80% wt/v, from about 0.05% wt/v to about 0.60% wt/v, from about 0.05% wt/v to about 0.40% wt/v, from about 0.05% wt/v to about 0.20% wt/v, or from about 0.05% wt/v to about 0.08% wt/v, such as about 3.2% wt/v. For example, the acylcarnitine is present in the ophthalmic composition in a concentration of at least about 0.05% wt/v, at least about 0.08% wt/v, at least about 0.10% wt/v, at least about 1.20% wt/v, at least about 1.40% wt/v, at least about 1.60% wt/v, at least about 1.80% wt/v, at least about 2.00% wt/v, at least about 3.20% wt/v, up to about 5.00% wt/v.

### B. Preservatives

The ophthalmic composition also includes a preservative in an effective amount to reduce or prevent microbial contamination. The preservative is typically one that, in the absence of a disclosed osmoprotective agent, cause hyperosmolarity in ophthalmic compositions and can lead to damage to the corneal epithelium. A non-limiting example of such a preservative is Benzalkonium chloride (BAC).

BAC is a quaternary ammonium cationic surface-acting agent and membranes and has a chemical formula C₆H₅CH₂N(CH₃)₂(CH₂)ₙCl, where n=8, 10, 12, 14, 16, or 18. The bactericidal activity of benzalkonium chloride is attributed, at least in part, to its ability to disrupt intermolecular interactions within bacterial cells. Such disruption is understood to interfere with the integrity of bacterial cell membranes by promoting the dissociation of lipid bilayers, thereby compromising membrane permeability and resulting in the uncontrolled leakage of intracellular components. In addition to membrane destabilization, benzalkonium chloride and other preservatives may also impair the structural conformation and function of macromolecules, including enzymes involved in cellular respiration and metabolic regulation in bacteria. These effects collectively inhibit biochemical processes by destabilizing specific intermolecular interactions and tertiary structures important for the survival and functionality of bacterial organisms. For these reasons, BAC is the most common preservative in ocular compositions, being present in approximately 70% of ophthalmic formulations (reviewed in Goldstein, et al., Nature Eye, 36, 361-368 (2022)).

Although BAC is a preferred preservative, the preservative included in the ophthalmic compositions is not limited to BAC. Other ophthalmically acceptable preservatives are also contemplated and may be used in place of or in combination with BAC. Such preservatives include, but are not limited to other quaternary ammoniums such as polyquaternium-1 (e.g., Polyquad®, Alcon Laboratories, Inc.), polyquaternium-42, and cetrimide; oxidizing agents such as sodium perborate (e.g., Genaqua®, Novartis Ophthalmics), stabilized oxochloro complex (e.g., Purite®, Bio-Cide International Inc.); ionic buffers such as borate, sorbital, propylene glycol, and zing (e.g., SofZia, Alcon Laboratories, Inc.); amidines such as chlorhexidine; parabens such as methylparaben and propylparaben; alcohols such as chlorobutanol and phenylethanol; and mercury-based chemicals such as thimerosal and phenylmercuric nitrate/acetate.

The concentration of preservative in the ophthalmic composition ranges from about 0.001% wt/v to about 0.02% wt/v. In some forms, the preservative is present in the ophthalmic composition in a range between about 0.001% wt/v to about 0.017% wt/v, between about 0.001% wt/v to about 0.015% wt/v, between about 0.001% wt/v to about 0.013% wt/v, between about 0.001% wt/v to about 0.010% wt/v, between about 0.001% wt/v to about 0.008% wt/v, between about 0.001% wt/v to about 0.006% wt/v, between about 0.001% wt/v to about 0.004% wt/v, or between about 0.001% wt/v to about 0.002% wt/v. For example, the preservative can be present in the ophthalmic composition in an amount that is at least about 0.001 wt/v %, at least about 0.003% wt/v, at least about 0.006 wt/v %, at least about 0.009 wt/v %, at least about 0.012 wt/v %, at least about 0.015 wt/v %, at least about 0.017 wt/v %, or at least about 0.02 wt/v %.

In some forms, the ophthalmic composition can include more than one preservatives such as BAC and a second quaternary ammonium compound e.g., polyquaternium-1. Polyquaternium-1 has an average molecular weight between about 200 g/mol and about 1,000 g/mol. When used, the second quaternary ammonium compound can be present in the composition in an amount that is greater than about 0.00001 w/v %, such as greater than about 0.0003 w/v % or such as greater than about 0.0007 w/v % of the ophthalmic composition.

### C. Additional Active Agents

The ophthalmic compositions can further contain one or more additional active agents to be delivered to the ocular surface. A biologically active agent is a substance used for the treatment (e.g., therapeutic agent), prevention (e.g., prophylactic agent), diagnosis (e.g., diagnostic agent), or to effect a cure or mitigation of disease or illness, alter the structure or function of the body, or pro-drugs, which become biologically active or more active after they have been placed in a predetermined physiological environment.

The one or more additional active agents can be one or more therapeutic agents, diagnostic agents, and/or prophylactic agents. The active agent can be a small molecule active agent and/or a biomolecule, such as an enzyme, protein, antibody, growth factor, polypeptide, polysaccharide, lipid, or nucleic acid. Suitable small molecule active agents include organic and organometallic compounds. In some forms, the small molecule active agent has a molecular weight of less than about 2000 g/mol, for example, less than about 1500 g/mol, less than about 1200 g/mol, less than about 1000 g/mol, or less than about 750 g/mol. In other forms, the small molecule active agent has a molecular weight less than about 500 g/mol. The small molecule active agent can be a hydrophilic, hydrophobic, or amphiphilic compound. Biomolecules typically have a molecular weight of greater than about 2000 g/mol and may include amino acids (such as peptide, proteins, and enzymes) or nucleic acids (such as siRNA, miRNA, oligonucleotides), sugars or polysaccharides, lipids, glycoproteins, or combinations thereof.

In some forms, the one or more additional active agents is an ophthalmic drug or compound. For example, the one or more additional active agents can be drug used to treat, prevent or diagnose a condition, disease or disorder of the eye. Non-limiting examples of ophthalmic drugs include anti-glaucoma agents, anti-angiogenesis agents, anti-infective agents, anti-inflammatory agents, growth factors, immunosuppressant agents, anti-allergic agents, complement inhibitors, and combinations thereof.

The one or more additional active agents, e.g., ophthalmic drug, can be present in the pharmaceutical composition in its neutral form, or in the form of a pharmaceutically acceptable salt. In some forms, it may be desirable to prepare a formulation containing a salt of an active agent due to one or more of the salt's advantageous physical properties, such as enhanced stability or a desirable solubility or dissolution profile. Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, Md., 2000, p. 704. Examples of ophthalmic drugs sometimes administered in the form of a pharmaceutically acceptable salt include timolol maleate, brimonidine tartrate, and sodium diclofenac.

### 1. Anti-Glaucoma Agents

In some forms, the one or more additional active agents is an anti-glaucoma agent. Exemplary anti-glaucoma agents include but are not limited to prostaglandin analogs (such as travoprost, bimatoprost, latanoprostene bunod, and latanoprost), beta-andrenergic receptor antagonists (such as timolol, betaxolol, levobetaxolol, and carteolol), alpha-2 adrenergic receptor agonists (such as brimonidine and apraclonidine), carbonic anhydrase inhibitors (such as brinzolamide, acetazolamide, and dorzolamide), miotics (i.e., parasympathomimetics, such as pilocarpine and ecothiopate), seretonergics muscarinics, dopaminergic agonists, and adrenergic agonists (such as apraclonidine and brimonidine).

### 2. Anti-Angiogenesis Agents

The ophthalmic composition can include one or more anti-angiogenesis agents. Exemplary anti-angiogenesis agents include, but are not limited to, antibodies to vascular endothelial growth factor (VEGF) such as bevacizumab (AVASTIN®) and rhuFAb V2 (ranibizumab, LUCENTIS®), and other anti-VEGF compounds such as aflibercept (EYLEA®), faricimab, and brolucizumab; MACUGEN® (pegaptanim sodium, anti-VEGF aptamer or EYE001) (Eyetech Pharmaceuticals); pigment epithelium derived factor(s) (PEDF), COX-2 inhibitors such as celecoxib (CELEBREX®) and rofecoxib (VIOXX®); interferon alpha; interleukin-12 (IL-12); thalidomide (THALOMID®) and derivatives thereof such as lenalidomide (REVLIMID®), squalamine; endostatin; angiostatin; ribozyme inhibitors such as ANGIOZYME® (Sirna Therapeutics); multifunctional antiangiogenic agents such as NEOVASTAT® (AE-941) (Aeterna Laboratories, Quebec City, Canada); receptor tyrosine kinase (RTK) inhibitors such as sunitinib (SUTENT®); tyrosine kinase inhibitors such as sorafenib (NEXAVAR®) and erlotinib (TARCEVA®); antibodies to the epidermal grown factor receptor such as panitumumab (VECTIBIX®) and cetuximab (ERBITUX®); as well as other anti-angiogenesis agents known in the art.

### 3. Anti-Infective Agents

The ophthalmic composition can include one or more anti-infective agents. Exemplary anti-infective agents include, but are not limited to, antiviral agents, antibacterial agents, antiparasitic agents, and anti-fungal agents.

Exemplary antiviral agents that can be included in the ophthalmic composition include but are not limited to idoxuridine, and trifluorouridine, ganciclovir and acyclovir.

In some forms, the one or more additional active agents include one or more antibacterial antibiotics. Exemplary antibiotics include, but are not limited to, aminoglycosides (e.g., streptomycin, amikacin, gentamicin, tobramycin, apramycin, arbekacin, bambermycins, butirosin, dibekacin, dihydrostreptomycin, fortimicin(s), isepamicin, kanamycin, micronomicin, neomycin, neomycin undecylenate, netilmicin, paromomycin, ribostamycin, sisomicin, spectinomycin, trospectomycin); ansamycins (e.g., geldanamycin, herbimycin, rifamide, rifampin, rifamycin SV, rifapentine, rifaximin); carbacephems (e.g., loracarbef); carbapenems (e.g., biapenem, imipenem, meropenem, panipenem); cephalosporins (e.g., cefaclor, cefadroxil, cefamandole, cefatrizine, cefazedone, cefazolin, cefcapene pivoxil, cefclidine, cefdinir, cefditoren, cefepime, cefetamet, cefixime, cefmenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, cefetecol, ceftibuten, ceftizoxime, ceftriaxone, cefuroxime, cefuzonam, cephacetrile sodium, cephalexin, cephaloglycin, cephaloridine, cephalosporin C, cephalothin, cephapirin sodium, cephradine, pivcefalexin); cephamycins (e.g., cefbuperazone, cefmetazole, cefminox, cefotetan, cefoxitin); monobactams (e.g., aztreonam, carumonam, tigemonam); β-lactams (e.g., oxacephems, flomoxef, moxalactam); penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, methampicillin, methicillin sodium, mezlocillin, nafcillin sodium, oxacillin, penamecillin, penethamate hydroiodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin); glycopeptides (e.g., vancomycin, teicoplanin, telavancin); lincosamides (e.g., clindamycin, lincomycin); macrolides (e.g., azithromycin, clarithromycin, dirithromycin, erythromycin, erythromycin acistrate, erythromycin estolate, erythromycin glucoheptonate, erythromycin lactobionate, erythromycin propionate, erythromycin stearate, carbomycin, josamycin, leucomycins, midecamycins, miokamycin, oleandomycin, primycin, rokitamycin, rosaramicin, roxithromycin, spiramycin, troleandomycin); polypeptides (e.g., bacitracin, colistin, polymyxin B, amphomycin, capreomycin, enduracidin, enviomycin, fusafungine, gramicidin S, gramicidins, mikamycin, polymyxins, pristinamycin, ristocetin, teicoplanin, thiostrepton, tuberactinomycin, tyrocidine, tyrothricin, viomycin, virginiamycin, zinc bacitracin); lipopeptides (e.g., daptomycin); tetracyclines (e.g., tetracycline, doxycycline, chlortetracycline, demeclocycline, minocycline, methacycline, lymecycline, oxytetracycline, clomocycline, apicycline, guamecycline, meclocycline, penimepicycline, pipacycline, rolitetracycline, sancycline); sulfonamides; nitrofurans; amphenicols (e.g., chloramphenicol, azidamfenicol, florfenicol, thiamphenicol); quinolones; and other antibacterial agents such as cycloserine, mupirocin, tuberin, and polyhexamethylene biguanide (PHEMB). These agents may be used singly or in combination, in amounts sufficient to exert a therapeutic effect against bacterial infections of the eye or surrounding ocular tissues.

In some forms, the one or more additional active agents include one or more synthetic antibacterial antibiotics. Examples of the synthetic antibacterials include, but are not limited to: 2,4-diaminopyrimidines (e.g., brodimoprim, tetroxoprim, trimethoprim), nitrofurans (e.g., furaltadone, fuirazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-b, chloramine-t, dichloramine t, n²-formylsulfisomidine, n⁴-β-d-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfonylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxine sodium, thiazolesulfone), and others (e.g., clofoctol, hexedine, methenamine, methenamine anhydromethylene-citrate, methenamine hippurate, methenamine mandelate, methenamine sulfosalicylate, nitroxoline, taurolidine, xibornol).

In some forms, the one or more additional active agents include one or more antifungal agents. Examples of the antifungal agents include, but are not limited to: polyenes (e.g., amphotericin b, candicidin, dennostatin, filipin, fungichromin, hachimycin, hamycin, lucensomycin, mepartricin, natamycin, nystatin, pecilocin, perimycin), others (e.g., azaserine, griseofulvin, oligomycins, neomycin undecylenate, pyrrolnitrin, siccanin, tubercidin, viridin). Examples of the synthetic antifungal agents include, but are not limited to: allylamines (e.g., butenafine, naftifine, terbinafine), imidazoles (e.g., bifonazole, butoconazole, clordantoin, chlormidazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole) others (e.g., acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, zinc propionate).

### 4. Anti-Allergic and Anti-Inflammatory Agents

The ophthalmic composition can include one or more anti-allergic agents. In some forms, the additional active agent is an anti-allergic agent such as olopatadine, epinastine, tranilast, ketotifen fumarate, pheniramine, diphenhydramine hydrochloride, and sodium cromoglicate.

In other forms, the one or more additional agents is an anti-inflammatory agent. Anti-inflammatory agents include both non-steroidal and steroidal anti-inflammatory agents. Suitable steroidal active agents include glucocorticoids, progestins, mineralocorticoids, and corticosteroids. Examples of the steroidal anti-inflammatory agents include, but are not limited to: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, and triamcinolone hexacetonide.

Examples of the non-steroidal anti-inflammatory agents include, but are not limited to: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprofen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (erg., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), E-acetamidocaproic acid, s-adenosylmethionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, and zileuton.

In some forms, the one or more additional active agents is a complement inhibitor. A variety of complement inhibitors are known in the art, and include, for example, C1-Inh (Cetor/Sanquin, BerinertP/CSL Behring, Lev Pharma), Rhucin/rhCl1NH (Pharming Group N.V.), sCR1/TP10 (Avant Immunotherapeutics), CAM-2/MLN-2222 (Millenium Pharmaceuticals), eculizumab/soliris (Alexion Pharmaceuticals), Pexelizumab (Alexion Pharmaceuticals), Ofatumumab (Genmab A/S), APL-2 (Apellis Pharmaceuticals), sDAF, sMCP, sMCP-DAF, sCDS9, DAF-cd59, C5a mutants, Antii-0S, Anti-C3, Anti-C3a, Anti-05a, NMeFKPdChaWdR, F-(OpdChaWR), Compstatin/POT-4 (Potentia Pharmaceuticals), OMS721 (Omeros Corporation), AMY-101 (Amyndas Pharmaceuticals SA), PMX-53 (Peptech Ltd.), rhMBL (Enzon Pharmaceuticals), the anti-C5 aptamer avacincaptad pegol/ARC1905 (Zimura®; IVERIC Bio [formerly, Ophthotech], Cranbury, NJ, USA), BCX-1470, FUT-175, K-76, thioester inhibitors, and factor D inhibitors such as ACH-4471 (Achillion Pharmaceuticals, Inc. (New Haven, Conn.)).

### 5. Anti-Neoplastic Agents and Diagnostic Agents

The ophthalmic composition can include one or more anti-antineoplastic agents. Examples of the antineoplastic agents include, but are not limited to: antineoplastic antibiotics and analogs (e.g., aclacinomysins, actinomycin f₁, anthramycin, azaserine, bleomycins, cactinomycin, carubicin, carzinophilin, chromomycins, dactinomycin, daunorubicin, 6-diazo-5-oxo-L-norleucine, doxorubicin, epirubicin, idarubicin, menogaril, mitomycins, mycophenolic acid, nogalamycin, olivomycins, peplomycin, pirarubicin, plicamycin, porfiromycin, puromycin, streptonigrin, streptozocin, tubercidin, zinostatin, zorubicin), antimetabolites exemplified by folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thioguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur).

Alternatively, one or more active agents in the ophthalmic composition is a diagnostic agent used for imaging or otherwise assessing the eye. Exemplary diagnostic agents include but are not limited to paramagnetic molecules, fluorescent compounds, magnetic molecules, radionuclides, x-ray imaging agents, and contrast media.

### D. Ophthalmic Formulations

Disclosed herein are ophthalmic formulations containing an effective amount of a preservative such as benzalkonium chloride and a osmoprotective agent, such as L-carnitine or a derivative or analog thereof, for administration to a subject in need thereof.

### 1. Pharmaceutically Acceptable Carriers and Excipients

The ophthalmic compositions are typically formulated with a pharmaceutically acceptable carrier suitable for topical ocular administration.

The term “pharmaceutically acceptable carrier” refers to any substance conventionally used in the pharmaceutical arts to facilitate the storage, administration, or therapeutic effect of a biologically active agent. The carrier is preferably in liquid form for topical delivery to the ocular surface. Non-limiting examples of suitable carriers for delivery to the ocular surface include but are not limited to oleoyl polyethylene glycol glyceride, linoleoyl glycol glyceride, lauroyl polyglycol glyceride, liquid paraffin, light liquid paraffin, soft paraffin (petrolatum), hard paraffin, wax, castor oil, peanut oil, sesame oil, medium chain triglycerides, cetearyl alcohol, lanolin, glycerol, propylene glycol, polyethylene glycol (PEG), and water.

“Ophthalmic solution” and “aqueous solution” include a homogeneous solution or a heterogeneous solution. Heterogeneous solutions include, but are not limited to, dispersions (e.g., oil dispersion in an aqueous solution such as emulsions), suspensions, and a combination thereof including other heterogeneous solutions where there are two or more visibly different substances or phases within the solution.

The formulations can be prepared using physiological saline as a vehicle. The pH of the formulation may be adjusted to a substantially neutral range, for example, approximately 7.4 or within the range of about 6.5 to about 7.4, using an appropriate buffer system such as acetate, citrate, phosphate, or borate buffers.

The ophthalmic formulations can additionally include a variety of pharmaceutical additives, including buffers, diluents, carriers, adjuvants, and excipients, to enhance the stability, bioavailability, and overall performance of the composition. Suitable buffering agents include, but are not limited to, tris and phosphate buffers. A range of other formulation components may be incorporated to serve specific functional roles. These may include co-solvents; surfactants such as Tween 80; oils; humectants and emollients; chelating agents; stabilizers; and antioxidants such as sodium metabisulfite, sodium thiosulfate, acetylcysteine, butylated hydroxyanisole, and butylated hydroxytoluene, viscosity modifiers, wetting agents, pH modifying agents, or combinations thereof. These additives may be selected and modified as needed based on the desired physicochemical properties and therapeutic application of the formulation. Suitable pharmaceutically acceptable excipients are preferably selected from materials which are generally recognized as safe (GRAS) and may be administered to an individual without causing undesirable biological side effects or unwanted interactions.

Any diluent used in the preparation of the formulation is preferably selected to preserve the biological activity of the composition. Suitable diluents include but are not limited to water, saline solutions, organic or inorganic salt solutions, Ringer's solution, dextrose solution, and Hank's solution.

Additional vehicles that can be used include but are not limited to polyvinyl alcohol, povidone, hydroxypropyl methylcellulose, poloxamers, carboxymethyl cellulose, hydroxyethyl cellulose, or purified water. Tonicity adjustors such as sodium chloride, potassium chloride, mannitol, or glycerin may also be used.

In some forms, the ophthalmic formulations further include a hydrogel. Exemplary hydrogels which can be used include, but are not limited to, hyaluronic acid, collagen, gellan, silk, fibrin, alginate, chitosan, polyacrylamides and methacrylate derivatives thereof, polyacrylic acid and methacrylate derivatives thereof, polyvinyl alcohol, polyethylene glycol and derivatives thereof, polypropylene glycol and derivatives thereof, or combinations thereof. For example, the hydrogel can include a hyaluronate derivative, for example poly (N-isopropylacrylamide) grafted sodium hyaluronate.

In some forms, the hydrogel has a polymer composition, for example a homopolymer, a copolymer, or combinations thereof. In some forms, the hydrogel contains one or more hydrophilic polymers, i.e., a polymer having at least 0.1 wt. % solubility in water, for example having at least 0.5 wt. % solubility. In some forms, the hydrophilic polymer has a solubility of at least 1 mg/mL. In some forms, the polymer composition can include one or more vinyl alcohol residues. In some forms, the polymer composition can include one or more acrylamide residues. In some forms, the polymer composition can include one or more residues selected from a polyethylene glycol derivative or a functionalized polyethylene glycol. In some forms, the polymer composition can include one or more acrylate residues or one or more methacrylate residues. In some forms, the polymer composition can include one or more residues selected from acrylamide, N-ornithine acrylamide, N-(2-hydroxypropyl) acrylamide, hydroxyethylacrylate, hydroxyethylmethacrylate, polyethyleneglycol acrylates, polyethyleneglycol methacrylates, N-vinylpyrrolidinone, N-phenylacrylamide, dimethylaminopropyl methacrylamide, acrylic acid, benzylmethacrylamide, methylthioethylacrulamide, or combinations thereof.

The indications, effective doses, formulations, contraindications, and other pharmacological attributes of these components are well known to those skilled in the art and may be selected and optimized as needed for the intended therapeutic use.

### 2. Viscosity, Osmolality, and pH

In some forms, the ophthalmic formulation can be in aqueous or non-aqueous forms and can further include one or more suspending agents and dispersing agents to promote and maintain a uniform dispersion of active and inactive components, for example, when the formulation is intended as a microparticulate suspension. Suitable suspending agents include, but are not limited to, polyvinyl pyrrolidone, glycerin monostearate, sorbitan esters (e.g., sorbitan monooleate, sorbitan monolaurate), and lanolin alcohols. Dispersing agents include surfactants such as tyloxapol and polysorbate 80, as well as ionic polymers such as sodium alginate, carboxymethyl cellulose, or hydroxypropyl methylcellulose. The concentration of suspending and dispersing agents can range from about 0.01% w/v to about 5% w/v, such as from about 0.05% w/v to about 2% w/v. In some forms, the concentration of suspending and dispersing agents can range from about 0.01% w/v to about 4.50% w/v, from about 0.01% w/v to about w/v 4.00%, from about 0.01% w/v to about 3.50% w/v, from about 0.01% w/v to about 3.00% w/v, from about 0.01% w/v to about 2.50% w/v, from about 0.01% w/v to about 2.00% w/v, from about 0.01% w/v to about 1.50% w/v, from about 0.01% w/v to about 1.00% w/v, from about 0.01% w/v to about 0.50% w/v, from about 0.01% w/v to about 0.20% w/v, or from about 0.01% w/v to about 0.10% w/v.

When the ophthalmic formulation is in the form of an aqueous or non-aqueous suspension or solution, or is presented as a gel or ointment, it is desirable to adjust the pH of the formulation to fall within a range that is compatible with the ocular surface to minimize irritation and maximize therapeutic effectiveness. Accordingly, the pH of the formulation can be adjusted to a range of about 4.0 to about 8.0, such as from about 6.5 to about 7.8, preferably from about 6.8 to about 7.5. In some forms, the ophthalmic formulation can have a pH from about 4.0 to about 7.5, from about 4.0 to about 7.0, from about 4.0 to about 5.5, from about 4.0 to about 5.0, from about 5.0 to about 8.0, from about 5.0 to about 7.0, from about 5.0 to about 6.0, from about 6.0 to about 8.0, or from about 6.0 to about 7.0. Suitable pH-modifying agents include ophthalmically acceptable acids and bases such as hydrochloric acid, sulfuric acid, boric acid, citric acid, sodium hydroxide, potassium hydroxide, and other pharmaceutically acceptable buffering agents known to those skilled in the art.

The osmolality may be adjusted to a range of about 150 mOsm/kg to about 500 mOsm/kg, more preferably from about 250 mOsm/kg to about 400 mOsm/kg, and most preferably from about 280 mOsm/kg to about 320 mOsm/kg, to facilitate compatibility with the natural tear film and to improve patient comfort and drug absorption. In some forms, the ophthalmic formulation has an osmolality ranging from about 150 mOsm/kg to about 450 mOsm/kg, from about 150 mOsm/kg to about 400 mOsm/kg, from about 150 mOsm/kg to about 350 mOsm/kg, from about 150 mOsm/kg to about 300 mOsm/kg, from about 150 mOsm/kg to about 250 mOsm/kg, from about 150 mOsm/kg to about 200 mOsm/kg, from about 200 mOsm/kg to about 450 mOsm/kg, from about 200 mOsm/kg to about 400 mOsm/kg, from about 200 mOsm/kg to about 350 mOsm/kg, or from about 200 mOsm/kg to about 300 mOsm/kg.

These components may be selected and modified based on the desired physicochemical properties, stability, and therapeutic application of the ophthalmic formulation. The formulation can be prepared under aseptic conditions and may be sterilized by filtration, autoclaving, or other suitable techniques known in the art.

### 3. Exemplary Ocular Formulations

The ophthalmic compositions can be formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine effective doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index, and is expressed as the ratio, LD₅₀/ED₅₀. Pharmaceutical compositions which exhibit large therapeutic indices are preferred. The data obtained from cell culture assays and animal studies can be used in formulating a range of dosages for human use.

The osmoprotective agent (e.g., L-carnitine or analog/derivative thereof), can optionally be added to existing or emerging BAC-containing ophthalmic compositions. In some forms, the osmoprotective agent (e.g., L-carnitine or an analog/derivative thereof) can be added to existing preservative-containing (e.g., BAC-containing) ophthalmic formulations with minimal or moderate modification to the base formulation, provided that the ratio of the osmoprotective agent to preservative is sufficient to reduce preservative-induced ocular surface damage (e.g., corneal epithelial disruption) without compromising the antimicrobial efficacy, stability (e.g., shelf life stability), and/or ocular surface permeability of the preservative. Alternatively, adjustments to the base formulation can be made to maintain stability, isotonicity, and pH balance of the ophthalmic composition.

i. Artificial Tears and Gel Drop Formulations

Ophthalmic formulations in the form of artificial tears or gel drops containing an effective amount of a preservative and an effective amount of an osmoprotective agent are described. These formulations can provide ocular surface hydration, maintain tear film stability, and reduce cellular stress in individuals experiencing dry eye symptoms, ocular surface disorders, or exposure to environmental irritants.

Artificial tears and gel drops are generally administered topically and are formulated to mimic the composition and function of natural tears. The formulations can be prepared for use as part of a daily eye care regimen, for frequent application throughout the day, or as long-acting gel drops intended to provide extended ocular surface retention. The compositions may be customized for therapeutic applications based on the needs of the intended user.

The artificial tear or gel formulations can be aqueous or semi-viscous in nature and may be suitable for multiple daily administrations or long-acting relief, depending on patient needs. The compositions may be applied directly to the ocular surface using standard ophthalmic delivery devices such as droppers, unit-dose pipettes, or preservative-free multidose systems. In some forms, the gel drops may provide improved ocular surface residence time due to their viscoelastic properties. For example, in some forms, the artificial tears or gel drops are provided in a fluid or semi-viscous form that is compatible with direct administration to the ocular surface using conventional ophthalmic droppers or unit-dose vials.

The disclosed formulations can be based on aqueous vehicles containing one or more hydrophilic polymeric agents, including but not limited to hyaluronic acid, carboxymethyl cellulose, hydroxypropyl methylcellulose, polyethylene glycol, polyvinyl alcohol, or other mucoadhesive and film-forming agents that promote retention on the ocular surface. In an aspect, the artificial tears or gel drops may include crosslinked or linear polymeric structures that form viscoelastic networks to enhance lubrication and protect the corneal epithelium. These polymers may also be combined with UV blockers, coloring agents, or stabilizers.

The artificial tear or gel drop formulations can be packaged in sterile, ophthalmic-grade containers such as single-use vials, preservative-free multidose bottles, or conventional droppers. The compositions may be stored in a suitable aqueous vehicle under aseptic conditions and may be sterilized using filtration, autoclaving, or other techniques commonly used in ophthalmic product manufacturing.

For application by the ophthalmic mucous membrane route, compositions may be formulated as eye drops or eye ointments. These formulations may be prepared by conventional means. Typically, the eye drops are in the form of an aqueous solution, for example, including one or more excipients suitable for administration into the eye. Most formulations applied topically as drops are administered as volumes of 0.03 mL/drop and 0.07 ml/drop, with a maximum typical volume of about 100 microliters. The formulations may also be provided in two units, one lyophilized and one a diluent for re-suspension of the lyophilized active agent(s).

For example, the ophthalmic formulations are in the form of anti-allergy eye drops containing an effective amount of a preservative and an effective amount of an osmoprotective agent. These formulations are intended to alleviate ocular symptoms associated with allergic conjunctivitis, including itching, redness, tearing, and irritation caused by seasonal allergens, dust, dander, or environmental pollutants. The anti-allergy eye drops can be used via direct topical administration to the ocular surface and can be used on an as-needed or scheduled basis. In some forms, the formulations are provided in aqueous form with appropriate viscosity-modifying agents to enhance retention time and maximize therapeutic efficacy. The compositions can be administered using standard ophthalmic dropper bottles, single-use vials, or preservative-free multidose systems. In these forms, the anti-allergy formulations can include one or more active antihistaminic or mast cell stabilizing agents such as olopatadine, ketotifen, azelastine, epinastine, or alcaftadine.

In another exemplary form, the ophthalmic formulations are in the form of eye drops containing an effective amount of a preservative, an effective amount of the osmoprotective agent, and an effective amount of an intraocular pressure-lowering agent are described. These compositions can be used for the management and treatment of glaucoma or ocular hypertension by reducing elevated intraocular pressure (IOP). For example, glaucoma eye drops can be formulated for topical administration and can be used once or multiple times daily depending on the specific agent and clinical protocol. The formulations may be presented as aqueous solutions or gel-forming liquids to enhance ocular surface retention and drug delivery efficiency. The formulations include one or more active agents such as prostaglandin analogs (e.g., latanoprost, bimatoprost, travoprost), beta-blockers (e.g., timolol, betaxolol), carbonic anhydrase inhibitors (e.g., dorzolamide, brinzolamide), alpha-2 adrenergic agonists (e.g., brimonidine), or fixed combinations thereof.

Exemplary gel drop formulations containing a preservative are provided in U.S. Pat. No. 9,533,053 to Gamache, et al., U.S. Pat. No. 8,791,154 to Gambache, et al., all of which are incorporated herein by reference in their entireties. Exemplary anti-glaucoma eye drop formulations are provided in U.S. Pat. Nos. 8,394,826; 8,450,344; 9,096,569; 9,415,043; 9,931,336; 10,174,017; 10,532,993; 10,588,901; 10,654,844; 10,882,840; 11,020,385; 11,021,456; 11,028,081; 11,185,538; and 11,618,748, all of which are incorporated herein by reference in their entireties. Exemplary eye drop formulations for reducing and/or preventing dry eye are provided in U.S. Pat. Nos. 8,685,945; 8,846,641; 10,004,685; 10,328,025; 10,624,848; 10,925,892; 11,234,929; 11,690,802; and 11,730,699, all of which are incorporated herein by reference in their entireties.

ii. Contact Lenses and Contact Lens Solutions

Ophthalmic formulations in the form of hydrogel contact lenses containing an effective amount of a preservative, and an effective amount of an osmoprotective agent are described. Extended wear contact lenses are usually soft contact lenses. They are typically made of flexible plastics that allow oxygen to pass through to the cornea. Fewer rigid gas permeable lenses have been designed and approved for overnight wear. Therefore, hydrogel contact lenses containing an effective amount of a preservative, and an effective amount of an osmoprotective agent can be for daily or overnight use, or for long-term use. The hydrogel contact lenses can be formulated for use in therapeutic applications according to the needs of the intended recipient. For example, in some forms, the hydrogel contact lenses have the same shape, size and contour as a contact lens, e.g., for use as a contact lens by direct application onto the surface of the eye. Thus, the disclosed formulations can be applied onto soft lenses made from one or more soft polymer materials including but not limited to, a hydrogel, a silicone-based hydrogel, a polyacrylamide, or a hydrophilic polymer. For example, in an aspect, contact lenses disclosed herein can include crosslinked hydrogels including hydrophilic monomers (e.g. N-Vinylpyrrolidone, N, N-dimethylacrylamide, 2-hydroxyethyl methacrylate, hydroxyethyl acrylate, methacrylic acid and acrylic acid), strengthening agents, ultraviolet light (UV) blockers, or tints. In another aspect, contact lenses disclosed herein can include silicone hydrogels (e.g. crosslinked hydrogels containing silicone macromers and monomers, as well as hydrophilic monomers that absorb water). In yet another aspect, contact lenses disclosed herein can include hard lenses made from one or more rigid materials including but not limited to, a silicone polymer, polymethyl methacrylate, or rigid gas permeable materials.

The disclosed hydrogel contact lenses can include one or more additional active agents. For example, the hydrogel contact lenses can include one or more therapeutic, diagnostic or prophylactic agents, or a dye, or coloring agent. The disclosed hydrogel contact lenses can be packaged and stored in appropriate containers, such as a contact lens case. The lenses can be stored within a suitable fluid, for example, contact lens solution.

In some forms, an effective amount of a preservative, and an effective amount of an osmoprotective agent, such as L-carnitine is formulated in a contact lens care solution to clean, disinfect, rinse, and store contact lenses. The solution can be provided as a sterile, aqueous composition suitable for use with soft hydrogel, silicone hydrogel, or rigid gas-permeable lenses. In one exemplary form, the formulation contains about 0.004% w/v BAC as a preservative, 0.15% w/v L-carnitine as an osmoprotective agent, 0.64% w/v sodium chloride and 0.075% w/v potassium chloride to adjust tonicity, 0.75% w/v boric acid and 0.12% w/v sodium borate as buffering agents, 0.05% w/v disodium EDTA to enhance preservative efficacy, and 0.2% w/v hydroxypropyl methylcellulose (HPMC) for improved lens wettability and ocular lubrication, with purified water as the vehicle. The solution can be used for daily cleaning, rinsing, and overnight storage of contact lenses and is compatible with conventional multidose packaging. Exemplary contact lenses formulations containing a preservative to which an osmoprotectant (e.g., L-carnitine) can be added, are provided in U.S. Pat. Nos. 4,525,346, 8,119,112, 8,759,321, 9,096,819, 8,106,151, and 8,309,679, which are all incorporated herein by reference in their entireties.

iii. Pre- and Post-Surgical Ophthalmic Preparations

Ophthalmic formulations in the form of pre- and post-surgical eye drops or gel preparations containing an effective amount of a preservative and an effective amount of an osmoprotective agent are described. These formulations are intended for use before and after ocular surgical procedures, including but not limited to cataract surgery, LASIK, corneal transplantation, and intravitreal injections. In these forms, the ophthalmic compositions can reduce the risk of infection, stabilize the ocular surface, and support tissue recovery during the perioperative period.

The formulations may be aqueous or gel-based and are designed for direct topical application to the ocular surface. They can be formulated for use in the hours or days leading up to a surgical procedure to prepare the eye, or administered postoperatively to assist with inflammation control, epithelial healing, and microbial prophylaxis. In some forms, the preparations are formulated for enhanced retention on the ocular surface, providing sustained release of active agents during critical phases of healing.

In certain forms, the formulations may include one or more additional active agents suitable for surgical applications. These may include broad-spectrum antibiotics (e.g., fluoroquinolones or aminoglycosides), corticosteroids or non-steroidal anti-inflammatory drugs (NSAIDs) to control inflammation, mydriatics or cycloplegics for intraoperative pupil dilation, as well as agents that promote wound healing or epithelial regeneration. Visualization agents or dyes may also be included to assist the surgeon in monitoring treatment zones or confirming delivery.

The pre- and post-surgical ophthalmic compositions can be supplied in sterile, single-dose vials or multidose containers, with or without preservative systems depending on the intended use and dosing frequency.

iv. Ocular Wound Bandage

Ocular wound bandages including the disclosed ophthalmic compositions are described. Ocular wound bandages including the ophthalmic compositions can be formulated for use in therapeutic or cosmetic applications, according to the needs of the intended recipient. In some forms, the ophthalmic are applied to commercially available ocular wound bandages, such as commercially available gauzes or eye patches. Application can occur after formulation of the ophthalmic composition has occurred, and can be carried out by any suitable means known in the art for application, such as spraying, coating, painting, etc.

In some forms, are applied in the form of a solution or powder directly onto gauze or other non-peptide structures. For example, the ophthalmic compositions and/or scaffold materials can be contacted with tissue around and within the eye, and held in place by a bandage or gauze, or as one component of an eye patch.

Medicated ocular bandages are reviewed in (Zidan, et al., Pharm Dev Technol, 23(3):255-260 (2017), discussed in brief below.

### Amniotic Membrane Bandages

Amniotic membrane (AM) is an avascular fetal membrane harvested from placental tissue obtained from elective cesarean sections of pregnant women.

### Bandage Contact Lenses (BCL)

A bandage lens is a therapeutic contact lens (usually soft) which can be worn eye for an extended period of time. A therapeutic bandage lens is any contact lens used to promote healing, relieve pain and protect the ocular surface. The placement of these therapeutic lenses, whether temporary or as part of a long-term treatment plan, should be thought of as a medical treatment or procedure, rather than a lens per se. It is a medical prosthesis for someone who has been injured, is necessary for the health of the eye (not for vision correction) and can minimize the risk of progressive disability, ocular morbidity or both. Commercial silicone hydrogel BCL including Bioaffinity®, ACUVUE®, PureVision®, and AIR OPTIX® are currently used after photorefractive keratectomy (PRK) to promote epithelial healing and control pain.

### Polymeric Hydrogels

Hydrogels are the main components of ocular bandage lenses because of their hydrophilic nature and cross-linked polymeric networks that have high water absorbance capability. Hydroxyethyl methacrylate (HEMA) and poly(hydroxyethylmethacrylate) (p-HEMA) are currently used in soft contact lens preparation and are the most common polymeric hydrogels utilized in drug-eluting contact lenses for controlled drug release up to 1 week.

### Collagen Shields

Collagen shields were first introduced in the late 80s as contact lens shaped bandages fabricated from porcine scleral tissue that resembles the collagen of the human eye now there are currently marketed collagen shields by OASIS® Medical, Inc.

v. Personal Eye Care and Eye Cosmetic Products

In some forms, the pharmaceutical compositions containing an effective amount of a preservative and an effective amount of an osmoprotective agent, such as L-carnitine, can be formulated for use in personal eye care and eye cosmetic products. These formulations can help to protect, soothe, and support the delicate tissues surrounding the eyes while delivering cosmetic or cleansing functionality. For example, the composition is incorporated into a mascara product to help maintain eyelid margin hygiene and prevent microbial contamination during daily wear. The mascara base can include film-forming agents, waxes, and pigments alongside the preservative and L-carnitine components, and may be packaged in standard mascara tubes with ophthalmically safe applicators.

Further, the compositions can be used in eye makeup removers to help minimizing ocular surface stress if they contact the ocular surface. These removers can be formulated as micellar solutions, dual-phase oil-in-water emulsions, or gel-based cleansers, and may include surfactants, emollients, and buffering agents alongside the preservative and osmoprotective ingredients to balance efficacy with ocular safety. Similarly, the pharmaceutical composition may be used in eyelid cleansers and scrubs formulated for daily or therapeutic use, particularly in individuals with blepharitis or meibomian gland dysfunction. These cleansers can be provided as foaming liquids, pre-moistened wipes, or gel-based applicators, and may include mild surfactants, humectants, and anti-inflammatory agents in combination with a preservative and osmoprotectants to cleanse the eyelid margin while maintaining tear film integrity.

Exemplary cosmetic formulations containing a preservative (such as cosmetic formulations for removing eye make-up) to which an osmoprotectant (e.g., L-carnitine) can be added. are provided in U.S. Pat. Nos. 4,252,826 and 4,732,692, all of which are incorporated herein in their entireties.

### III. Methods of Using Ophthalmic Formulations

Methods of administering ophthalmic formulations containing an effective amount of an osmoprotective agent to the ocular surface for the support and preservation of corneal epithelial integrity and the reduction of hyperosmolarity-related stress are provided. In some forms, although not intended to treat or cure an underlying ocular disease eliciting treatment with an original formulation, the addition of an improved formulation including an osmoprotective agent can help prevent or reduce secondary ocular surface damage that may result from environmental exposure, injury, or inflammatory conditions. Thus, in any of the methods, the effect or results obtained by the disclosed formulation can be compared to the results obtained by the same formulation in the absence of the osmoprotective agent (e.g., L-carnitine or a derivative/analog thereof).

Furthermore, it will also be appreciated that the although the ophthalmic formulations are typically discussed herein as including both BAC and an osmoprotective agent (e.g., L-carnitine or a derivative/analog thereof), these two agents can also be administered separately in the disclosed methods. For example, in some forms BAC and an osmoprotective agent are administered separately. The separate administration can be concurrent or sequential (i.e., osmoprotective agent-containing solution followed by a BAC-solution; or BAC-containing solution followed by an osmoprotective agent; or a combination thereof). Thus, in some forms, the ophthalmic formulations of each of the disclosed methods is two solutions separately administered. Therefore, all of the methods discussed herein are specifically disclosed as administering and/or treating the subject with a single formulation containing BAC and an osmoprotective agent, and by administration of the two foregoing agents in separate solutions.

Ocular surface stress and epithelial disruption can arise in various clinical contexts, including post-injury recovery, exposure to desiccating environments, or as a result of extended contact lens wear. Timely application of formulations containing osmoprotective agents such as L-carnitine can mitigate tear film instability, reduce cellular stress responses, and promote epithelial recovery.

Ocular care protocols for surface injuries, chemical exposure, or epithelial breakdown emphasize rapid evaluation, stabilization, and protection of the eye. In such cases, formulations containing osmoprotective agents can be self-administered or applied during first response care to help prevent further dehydration, irritation, or mechanical damage. These agents contribute to the creation of a surface microenvironment that reduces osmotic stress and supports tissue viability, facilitating natural repair mechanisms in the corneal epithelium and surrounding ocular surface. For example, the ophthalmic formulations can be adapted for long-term use helping maintain tear film balance, prevent ocular surface inflammation, and support visual function by preserving the epithelial barrier.

In some forms, the ophthalmic formulations are administered to a subject in need thereof, in an amount effective to decrease or prevent preservative-induced (e.g., BAC-induced) ocular surface damage, i.e., that may occur in the absence of the osmoprotective agent. In some forms, the ocular surface damage may include one or more, but is not limited to, increased corneal epithelial disruption, loss of adherens junction integrity, and tear film instability. As demonstrated in the non-limiting Examples, administration of an effective amount of a composition containing L-carnitine and BAC significantly improved corneal epithelial barrier function and reduced BAC-induced ocular surface damage. In vitro, the composition containing L-carnitine restored transepithelial resistance and adherens junction protein expression in stressed corneal epithelial cells (see Example 1). In vivo the composition containing L-carnitine preserved corneal clarity, reduced epithelial defects, restored tear production, and increased adherens protein expression.

### A. Administration

The disclosed ophthalmic formulations can be administered via any means which is effective to deliver an effective amount of the preservative and the osmoprotective agent to the eye (e.g., the ocular surface). For example, compositions can be administered by topical, intra-corneal, periocular, subconjunctival routes. In preferred forms, the formulations are administered topically to the ocular surface such as to the conjunctival sac (i.e., the space between the eyelid and eyeball).

The ophthalmic formulations can be applied to the outer surface of the eye, or tissues contacting or surrounding the eye to be treated. Exemplary tissues that can be contacted with the ophthalmic formulations include the lids, lacrimal glands, lacrimal ducts, conjunctiva, sclera, cornea, aqueous humor, iris, ciliary body, trabecular meshwork, and lens. Exemplary ocular compartments into which material can be administered include the episclera, the conjunctiva, the sclera or the anterior chamber, and the cornea and compartments therein (e.g., subepithelial, intrastromal, endothelial).

The ophthalmic formulations containing an effective amount of a preservative and an osmoprotective agent can be administered as a liquid (e.g., eye drops). In other forms, the ophthalmic formulations can be administered to the eye as an emulsion (e.g., ocular spray), for example, as a mixture of solid particles including an effective amount of a preservative and an osmoprotective agent.

The ophthalmic formulations containing an effective amount of a preservative and an effective amount of an osmoprotective agent can be delivered to the ocular surface using conventional topical administration techniques. For example, the ophthalmic formulations described herein can be applied to the external structures of the eye, such as the cornea, conjunctiva, and eyelid margins, without breaching the ocular barrier.

In some forms, the ophthalmic formulation is applied topically using mechanical delivery means, including dropping, spraying, or bathing the ocular surface in a solution, or a combination thereof. These methods allow direct exposure of the formulation to the tear film and corneal epithelium. When administered as drops, the volume per application generally ranges from about 0.03 mL to about 0.07 mL per drop, with a maximum of approximately 100 microliters per drop. The formulation may be dispensed from conventional ophthalmic dropper bottles, single-use sterile vials, or multidose containers with preservative systems suitable for repeated application.

In some forms, the ophthalmic formulation containing an effective amount of a preservative and an effective amount of an osmoprotective agent can be administered to the ocular surface in a powder form using a non-invasive topical delivery method. For example, a dry powder formulation may be applied to the external surface of the eye using a calibrated air applicator, which can be adjusted to allow for targeted delivery to a localized region or broader application across the entire exposed ocular surface. This method permits gentle, directed administration without direct contact or penetration of the ocular tissue. In other forms, a device or applicator can be used to mix two or more components of the formulation immediately prior to or during application, allowing staged or sequential delivery to the ocular surface. This approach can involve delivering individual components of the formulation in a specific sequence, such as a powder followed by a liquid, and then another powder, thereby forming layered or structured deposits that adapt to the shape and moisture conditions of the eye.

For therapeutic administration, the ophthalmic formulations can be applied to a subject experiencing ocular surface discomfort, epithelial instability, or tear film irregularities. For example, the formulation may be administered to individuals exhibiting symptoms such as dryness, irritation, or sensitivity commonly associated with ocular surface stress. Typically, the subject is a human. Accordingly, methods of using the ophthalmic formulation may include identifying or selecting a subject who may benefit from enhanced corneal epithelial integrity or improved tear film stability. An exemplary subject is a human patient experiencing ocular surface challenges resulting from environmental exposure, prolonged screen time, aging, or contact lens wear. These formulations are intended to support ocular surface health rather than treat or cure an underlying disease.

In some forms, the subject is a patient with one or more underlying medical conditions. Exemplary underlying medical conditions include metabolic diseases, autoimmune diseases, immuno-deficiencies, infectious diseases, cancer, and neurological diseases. In some forms, the subject is a patient undergoing therapy for an underlying medical condition. Therefore, in some forms, the subject can have one or more diseases or disorders of the eye that are associated with or caused by a therapeutic intervention. Exemplary therapeutic interventions include chemotherapy, anti-retroviral therapy, anti-coagulant therapy, antihistamine medication, decongestant medication, hormone replacement therapy, antidepressant medication, drugs for high blood pressure, acne medication, birth control and medication for Parkinson's disease.

For prophylactic administration, the ophthalmic formulations can be administered to a subject at risk of developing a particular ocular condition or disorder (e.g., dry eye disease). Alternatively, prophylactic administration can be applied to avoid the onset of symptoms in a patient diagnosed with the underlying condition or disorder.

The ophthalmic formulations can be provided in a form that allows for topical self-administration by the patient and may exist in various physical states before, during, or after application to the ocular surface. For example, the formulation may be manufactured in a gelled state through exposure to an ionic solution and stored in that form until use. In some forms, the gel can be dehydrated for stability during storage and rehydrated upon administration. The formulation can also be molded into a specific shape, such as a thin layer designed for application to the eye or to coat the surface of a soft contact lens.

### B. Ocular Diseases and Conditions

The disclosed ophthalmic compositions can be used to reduce and/or prevent the progression of a variety of ocular surface conditions, such as those involving epithelial barrier disruption, inflammation, or tear film instability. For example, the compositions can be used in the treatment or management of conditions affecting the eyelids and lid margin, such as blepharitis, meibomian gland dysfunction, and ocular rosacea; conditions of the conjunctiva, including allergic conjunctivitis, irritative conjunctivitis, and ocular surface inflammation associated with dry eye disease; and conditions of the cornea, such as superficial punctate keratitis, keratoconjunctivitis sicca, corneal epithelial defects, and contact lens-related discomfort. In some forms, although not intended to treat or cure the underlying ocular disease for which the original formulation is administered, the addition of an osmoprotective agent to the composition can help prevent or reduce secondary ocular surface damage resulting from environmental stress, injury, or inflammation. Accordingly, the efficacy of the disclosed formulation can be assessed by comparison to the same formulation lacking the osmoprotective agent (e.g., L-carnitine or a derivative/analog thereof).

The ophthalmic formulations, optionally including one or more additional therapeutic agents, can be used to treat or prevent any eye diseases or disorders according to the methods.

Symptoms of exemplary eye diseases and disorders that can be treated include keratitis, conjunctivitis, dry eye disease and recurrent corneal erosion syndrome, allergic conjunctivitis, exposure keratopathy, amoebic keratitis, fungal keratitis, bacterial keratitis, viral keratitis, onchocercal keratitis, bacterial keratoconjunctivitis, viral keratoconjunctivitis, corneal dystrophic diseases, Fuchs' endothelial dystrophy, Stevens-Johnson syndrome, corneal neovascularization diseases, post-corneal transplant rejection prophylaxis and treatment, autoimmune uveitis, infectious uveitis, anterior uveitis, posterior uveitis (including toxoplasmosis), pan-uveitis, an inflammatory disease of the vitreous or retina, endophthalmitis prophylaxis and treatment, macular edema, macular degeneration, age related macular degeneration, corneal ectasia, keratoconus proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof. In some forms, the methods are effective to treat and prevent one or more symptoms of an acute eye disease or disorder. In some forms, the methods are effective to treat and prevent one or more symptoms of a chronic eye disease. In other forms, the methods are effective to treat and prevent one or more symptoms or disorders of the eye resulting from a traumatic or surgical injury. Therefore, the methods can be used to treat and prevent conditions of the surface of the eye or one or more of the interior compartments of the eye resulting from chemical burns, heat burns, surgery, injury, or other damage resulting to exposure to one or more environmental or chemical agents.

The methods can be prophylactic, for example, to prevent the onset or development of a disease or disorder of the eye. In some forms, the methods include treating an individual who has not been diagnosed with one or more diseases or disorders of the eye, but who has been identified as being at risk of developing or acquiring one or more diseases, disorders or injuries of the eye. Exemplary subjects at risk of developing or acquiring one or more diseases, disorders or injuries of the eye include subjects with metabolic disease, immunocompromised subjects and subjects with infectious diseases or at risk of developing infectious diseases. In some forms, the methods are used to treat or prevent diseases in diabetic subjects. In other forms, the methods are used to treat or prevent diseases in patients prior to, during or after one or more investigative or surgical procedures of the eye.

### 1. Ocular Inflammation

Methods of administering the ophthalmic compositions to one or more of the structures of the eye for treating and preventing ocular inflammation and/or intraocular inflammation are provided. In some forms, the ophthalmic compositions are administered to a subject in need thereof, to reduce, treat, and/or prevent inflammatory responses within or around the eye. In some forms, the formulations are administered to a subject in need thereof, to reduce, manage, or prevent symptoms of secondary ocular surface damage due to preservative-induced hyperosmolarity (e.g., corneal opacity) that occurs as a consequence of treatment for ocular inflammation.

Suppression of unwanted inflammation resulting from disease and/or injury can be of high clinical importance in many environments. Care protocols for ophthalmic injuries and diseases indicate that the most important medical actions for an ocular injury are evaluation of the extent of the injury, evacuation of the subject for more specialized treatment, and immediate protection of the eye from further damage.

Ocular inflammation, whether due to allergic conjunctivitis, blepharitis, dry eye disease, or post-surgical irritation, often leads to increased tear film osmolarity, epithelial disruption, and discomfort. Formulations containing osmoprotective agents such as L-carnitine, can reduce cellular stress by restoring osmotic balance across the epithelial membrane. This osmoprotection can mitigate inflammation-driven exacerbation of epithelial damage and promote conditions that support epithelial repair and barrier function.

In clinical and home settings, early and consistent topical administration of these formulations may help maintain ocular surface homeostasis and reduce discomfort associated with inflammatory processes. By creating a protective microenvironment on the ocular surface, these formulations may limit the exposure of inflamed tissues to mechanical friction, environmental irritants, and hyperosmolar tears. This supportive role is especially important in patients undergoing treatment for ocular inflammation with agents such as corticosteroids or immunomodulators, where maintaining epithelial integrity can improve comfort and therapeutic outcomes.

In some forms, the formulations may be used adjunctively during flare-ups or following exposure to inflammatory triggers, acting to soothe the ocular surface and reduce the risk of epithelial degradation. The use of osmoprotective agents in this context may contribute to limiting inflammation-related complications by promoting surface stability, reducing epithelial cell stress, and supporting the natural resolution of inflammation.

### 2. Dry Eye Disease (DED)

The ophthalmic compositions can be administered to a subject in need thereof, to reduce, treat, and/or prevent dry eye disease and/or symptoms associated therewith. In some forms, the formulations are administered to a subject in need thereof, to reduce, manage, or prevent symptoms of secondary ocular surface damage due to preservative-induced hyperosmolarity (e.g., corneal opacity) that occurs as a consequence of treatment for dry eye disease and/or symptoms associated therewith.

Dry Eye Disease (DED) (also Keratoconjunctivitis Sicca; KCS) is a multifactorial disease of the tears and ocular surface that may cause discomfort, visual disturbance, tear film instability and damage to the ocular surface. The term “dry eye” is synonymous with “keratoconjunctivitis sicca” (KCS) (Lemp, et al., Report of the International Dry Eye WorkShop (DEWS) Ocul Surf 5: 65-204 (2007)). Dry eye is a common ocular disorder which afflicts tens of millions of people globally. DED is associated with a range of symptoms and is often further described according to its underlying cause, including for example, autoimmune dry eye diseases, environmental dry eye diseases, age-related dry eye disease, computer-related dry eye, and dry eye following Lasik, dry eye associated with reading, driving or watching a movie or television, etc.

The lives of people with chronic DED are negatively impacted due to persistent eye pain, redness and/or dryness. Typically, DED is accompanied by increased osmolarity of the tear film and inflammation of the ocular surface. In addition, adhesions may form between the palpebral conjunctiva of the eyelids and the corneal epithelium in dry eye patients, causing recurrent pain and further damage to the corneal epithelium.

In some forms, the ophthalmic formulations are effective to treat and prevent one or more symptoms of dry eye disease by reducing the evaporation of tears or tear film, preventing the formation of adhesions of the corneal epithelium, providing a continuous surface for the maintenance of the tear film, enhancing or inducing healing of the damaged corneal tissue, reducing inflammation of the cornea, reducing irritation and pain, or combinations of these.

Dry eyes are a common symptom of Sjogren's syndrome. Therefore, in some forms, the ophthalmic formulations are effective for the treatment and prevention of dry eyes associated with Sjogren's syndrome.

### 3. Recurrent Corneal Erosion Syndrome (RCES)

In some forms, the ophthalmic compositions can be administered to a subject in need thereof, to reduce, treat, and/or prevent Recurrent Corneal Erosion Syndrome (RCES), and/or symptoms associated therewith. In some forms, the formulations are administered to a subject in need thereof, to reduce, manage, or prevent symptoms of secondary ocular surface damage due to preservative-induced hyperosmolarity (e.g., corneal opacity) that occurs as a consequence of treatment for RCES and/or symptoms associated therewith.

Recurrent corneal erosion syndrome (RCES) is a common clinical disorder characterized by a disturbance at the level of the corneal epithelial basement membrane, resulting in defective adhesions and recurrent breakdowns of the epithelium. Pain associated with RCES, whether due to trauma or to anterior basement membrane dystrophy (e.g., Cogan dystrophy or map dot fingerprint dystrophy), results from abnormalities in the epithelial basement membrane. Additional complications, such as adhesions between the palpebral conjunctiva of the eyelids and the corneal epithelium in dry eye patients contribute significantly to RCES in many patients.

Corneal erosions are common chronic ocular disorders and can worsen with time, especially if neglected. RCES may occur either secondary to corneal injury or spontaneously. When RCES arises spontaneously, a predisposing factor, such as corneal dystrophy, diabetes, or infection, may be the underlying cause. Management of RCES is usually aimed at regenerating or repairing the epithelial basement membrane to restore the adhesion between the epithelium and the anterior stroma.

Painful RCES, whether due to trauma or to anterior basement membrane dystrophy (Cogan dystrophy or map-dot-fingerprint dystrophy), results from abnormalities in the epithelial basement membrane. Recurrent corneal erosions and epithelial basement membrane dystrophy are usually bilateral and are characterized by various patterns of dots, parallel lines that mimic fingerprints, and patterns that resemble maps, which appear in the epithelium. Individual microcysts may be oval, oblong, or comma-shaped and rarely appear alone but usually are associated with map and fingerprint patterns. On the other hand, the map and fingerprint patterns appear without dots or individual microcysts. Map and fingerprint alterations of the corneal epithelium are not rare and can be found in asymptomatic individuals without prior history of trauma or ocular disease. Epithelial changes are more common than previously recognized and are frequently present in conditions involving corneal edema (e.g., such as near a healing cataract surgery incision) or in the center of the cornea associated with Fuchs corneal dystrophy.

The epithelial healing process begins when basal epithelial cells undergo mitosis, producing new cells that occupy fresh wounds. Basal cells adhere the epithelium to the stroma in two ways: they secrete the basement membrane, and they contain hemidesmosomes, which behave as linchpins that protrude through the posterior surface of basal cells and into the stroma; each is held in place by an anchoring fibril. Any disruption to basal cell production makes the eye more prone to recurrent erosion.

Recurrent corneal erosions occur because of a defect in the epithelial basement membrane and in hemidesmosomes formation, resulting in epithelial loss, microcysts, and bullae. Recurrent corneal erosions occurring after injury or any corneal insult typically result from improper or inadequate healing of the basement membrane, typically either because the basal epithelial cells fail to produce proper basement membrane complexes to attach to the Bowman layer and stroma or because of faulty basement membrane adherence.

A traumatic cause has a better eventual prognosis for full recovery than does the spontaneous form. In the case of spontaneous corneal erosions, the underlying disease process may be an epithelial basement membrane corneal dystrophy. Electron microscope studies have shown that during recurrent corneal erosion episodes, there is separation of the anchoring system at the level of the epithelial cell membrane or below the level of the anchoring plaques. Normal and degenerate polymorphonuclear leucocytes (PMNs) were found within and between the epithelial cells and within the anchoring layer. The degenerate PMNs may secrete metalloproteinases that cleave the Bowman layer below the anchoring system.

In some forms, the ophthalmic formulations are effective to treat and prevent one or more symptoms of recurrent corneal erosion syndrome by reducing the evaporation of tears or tear film, preventing the formation of adhesions of the corneal epithelium, providing a continuous surface for the maintenance of the tear film, enhancing or inducing healing of the damaged corneal tissue, reducing inflammation of the cornea, reducing irritation and pain, or combinations of these. In a particular form, the ophthalmic formulations are effective to treat and prevent one or more symptoms of recurrent corneal erosion syndrome by enhancing or inducing the regeneration or repair of the epithelial basement membrane to restore the adhesion between the epithelium and the anterior stroma. This enhancement can be achieved through forming a barrier to the passage of inflammatory cells and markers, and preventing access of inflammatory factors, such as microbial or environmental contaminants to the damaged corneal surface.

### 4. Fuchs Dystrophy

In some forms, the ophthalmic compositions can be administered to a subject in need thereof, to reduce, treat, and/or prevent Fuch's Dystrophy, and/or symptoms associated therewith. In some forms, the formulations are administered to a subject in need thereof, to reduce, manage, or prevent symptoms of secondary ocular surface damage due to preservative-induced hyperosmolarity (e.g., corneal opacity) that occurs as a consequence of treatment for Fuch's Dystrophy and/or symptoms associated therewith.

Fuch's Dystrophy (also known as Fuchs' corneal endothelial dystrophy or FCED) is a slowly progressing corneal dystrophy that usually affects both eyes. FCED results in corneal clouding, loss of corneal sensation and the formation of epithelial bullae.

Multiple stages of Fuchs endothelial dystrophy are recognized, typically evolving gradually over a period of about 25 years, and, like other corneal dystrophies, they usually are bilateral but asymmetric.

The first stage is the onset of cornea guttata, usually in the fourth decade of life. Subjective symptoms rarely occur until the fifth or sixth decade. During the asymptomatic phase, endothelial guttata and pigment dusting can be seen by slit lamp examination of the central corneal endothelium and by specular reflection. The guttate excrescences can become more numerous and confluent so that individual guttata are lost completely in the beaten-metal appearance of the endothelial surface. The central cornea is involved first, and, as the disease progresses, it spreads toward the periphery.

In the second phase of the disease, blurred vision, glare, and halos around lights develop because of incipient corneal edema in the stroma and epithelium. Epithelial edema can be seen as small droplets (bedewing) on retroillumination with the slit lamp. Epithelial microcysts coalesce to form bullae, which produce varying amounts of pain when they burst; hence, the name bullous keratopathy. Striae form in the Descemet membrane as the cornea thickens posteriorly due to stromal swelling. The arc of the Descemet membrane from limbus to limbus is shortened, causing wrinkles in the Descemet membrane called striae. The microcystic epithelial vesicles may break, causing foreign body sensations and severe pain with more extensive corneal epithelial disruption.

In the third stage, recurrent corneal erosions, microbial ulceration, and persistent pain may occur. Corneal sensitivity usually is reduced.

### 5. Eye Infections

In some forms, the ophthalmic compositions can be administered to a subject in need thereof, to reduce, treat, and/or prevent eye infections and/or symptoms associated therewith. In some forms, the formulations can be administered to a subject in need thereof, to reduce, manage, or prevent symptoms of secondary ocular surface damage due to preservative-induced hyperosmolarity (e.g., corneal opacity) that occurs as a consequence of treatment for eye infections and/or symptoms associated therewith. For example, the formulations can be administered to a subject in an amount effective for reducing and/or treating hyperosmolarity that occurs as a consequence of treatment for ocular infections, such as hyperosmolar stress induced by preservatives in anti-infective ophthalmic medications. Therefore, in some forms, the ophthalmic formulations are used to treat or prevent eye infections from bacteria, fungi, viruses, and protozoa. Eye infections can occur in different parts of the eye and can affect just one eye or both. Exemplary symptoms associated with eye infections include conjunctivitis, stye, inflammation and pain.

In some forms, the formulations are for prophylactic purposes to prevent an outset of a suspected eye infection. For example, if one person with an eye infection, e.g., conjunctivitis, is identified, anyone who has been recently in contact with that person can use the disclosed formulation for prophylactic purposes. In some forms, the disclosed formulations are used to prevent, reduce, or alleviate one or more symptoms from an eye infection.

In some forms, the ophthalmic formulations are used to treat or prevent eye keratitis, i.e., inflammations of the cornea, by applying the ophthalmic formulations onto the corneal stroma. Conditions treated by the methods include, but are not limited to, amoebic keratitis, fungal keratitis, bacterial keratitis, viral keratitis, and onchorcercal keratitis.

In some forms, the ophthalmic formulations are used to treat or prevent Amoebic keratitis. Amoebic keratitis can be caused by acanthamoeba and can be treated by administering to the corneal stroma of a subject in need of treatment thereof the compositions, optionally containing one or more anti-amoebic agents. Any anti-amoebic agent known in the art can be combined with the compositions. Representative, anti-amoebic agents include, but are not limited to, polyhexamethylene biguanide (PHMB), propamidine isethionate, miconazole nitrate, neomycin, chlorhexidine digluconate, polymicin B, clotrimazole, and combinations thereof.

In some forms, the ophthalmic formulations are used to treat or prevent fungal keratitis. Fungal keratitis can be caused by, for example, aspergillus fumigates, fusarium, and/or yeasts, such as candida, and can be treated by administering to the corneal stroma of a subject in need of treatment thereof the compositions, optionally containing one or more anti-fungal agents. Any anti-fungal agent known in the art can be combined with the compositions. Representative, anti-fungal agents include, but are not limited to, natamycin, nystatin, amphotericin B, chlorhexidine, fluorinated pyrimidines, such as flucytosine, azoles, such as imidazoles and triazoles, including, but not limited to, ketoconazole, miconazole, itraconazole, fluconazole, econazole, and clotrimazole, and combinations thereof.

In certain forms, the ophthalmic formulations are used to treat or prevent bacterial keratitis. Bacterial keratitis can be caused, for example, by Streptococcus, Pseudomonas, Enterobacteriaceae (including Klebsiella, Enterobacter, Serratia, and Proteus), and Staphylococcus species. Such infections can be treated by administering to the corneal stroma of a subject in need of treatment thereof the compositions, optionally containing one or more anti-bacterial agents. Further, up to 20% of cases of fungal keratitis (particularly candidiasis) can be complicated by bacterial co-infection. Thus, in some forms, the methods include combination therapies involving administering to the corneal stroma of a subject in need of treatment thereof the compositions, optionally containing one or more anti-fungal agents and one or more anti-bacterial agents.

In certain forms, the ophthalmic formulations are used to treat or prevent viral keratitis. Viral keratitis can, for example, be caused by a herpes simplex virus and can be treated by administering to the corneal stroma of a subject in need of treatment thereof the compositions, optionally containing one or more anti-viral agents. Any anti-viral agent known in the art can be combined with the compositions. A representative anti-viral agent includes, but is not limited to, acyclovir.

In some forms, the ophthalmic formulations are used to treat or prevent keratitis associated with infection by a nematode, such as onchoceral keratitis. Onchoceral keratitis, also referred to as “River Blindness,” is caused by the nematode Onchocerca volvulus and can be treated by administering to the corneal stroma of a subject in need of treatment thereof the compositions, optionally containing one or more anti-parasitic agents. Any anti-parasitic agent known in the art can be combined with the compositions. A representative anti-parasitic agent includes, but is not limited to, ivermectin.

### 6. Drug-Induced Eye Conditions

The formulations are suitable for reducing and/or treating hyperosmolarity that occurs as a secondary consequence of treatment for drug-induced eye conditions. Thus, in some forms, the formulations are used prior, during or after taking one or more systemic drugs. In some forms, the ophthalmic formulations can be administered into or onto the eye prophylactically, to prevent disruption of the tear film or tear production in a subject identified at risk of disruption of the tear film or tear production. Therefore, methods of administering the ophthalmic formulations into or onto the eye for treating and preventing eye diseases and disorders in subjects who have taken or are prescribed to take one or more of therapeutic agents associated with disruption of the tear film or tear production are provided. Exemplary drugs that can cause ocular side effects include corticosteroids, antihistamines, antipsychotic medications, anti-malarials, blood pressure medications, herbal medicines, erectile dysfunction drugs, anticholinergics, immuno-suppressants, antibiotics, antiarrhythmic agents, and anti-cancer drugs/treatment. Some specific examples are bisphosphonate, amiodarone, tamsulosin, topiramate, ethambutol, minocycline, cyclosporine and tacrolimus.

Corticosteroids used for many conditions, such as asthma, allergies, arthritis and dermatitis, can cause swelling in the back of the eye or retina and potentially lead to cataracts. Antihistamines, used for conditions such as allergies, can raise certain patients' risk for glaucoma. Antipsychotic medications, such as THORAZINE® and MELLARIL® can be toxic to the retina. Anti-malarials, such as PLAQUENIL® (hydroxychloroquine), used to treat malaria, lupus and rheumatoid arthritis, is a known retinal toxin, and the effects are irreversible. FOSAMAX®, a bisphosphonate that is prescribed for post-menopausal women to prevent calcium bone loss, can cause orbital inflammation, uveitis and scleritis.

Cyclosporine and Tacrolimus, commonly used in patients who have undergone organ or bone marrow transplants, can cause posterior reversible encephalopathy syndrome. These patients present with bilateral vision loss. Minocycline is a tetracycline derivative commonly used to treat acne. Minocycline can cause increased intracranial pressure and papilledema, which can cause permanent vision loss if not reversed. Ethambutol is widely used to treat mycobacterial disease, including tuberculosis; if not taken at safe doses, it is an optic nerve toxin. Topiramate (Topamax) is used to treat epilepsy and migraine headaches, and it is used off-label for weight loss. It can cause angle-closure glaucoma soon after starting treatment. Tamsulosin (Flomax), which is used to treat prostate enlargement and improve urinary flow in men. The well-known syndrome, intraoperative floppy iris syndrome, is often associated with men who were on medicine to relax their prostate. Women with these drugs can at the time of cataract surgery, make surgical risk much higher. Amiodarone (Cordarone) effectively treats cardiac arrhythmias. It causes the appearance of a whorl in the cornea, which does not usually cause symptoms, although some people can have a little bit of blurred vision.

Anticholinergics e.g., dicyclomine (BENTYL®), and other drugs with anticholinergic effects, are administered to patients who have stomach conditions that require stomach relaxers and to patients with Parkinson's disease. Young patients taking these drugs will develop difficulty with accommodation. Erectile dysfunction drugs, e.g., sildenafil citrate (VIAGRA®) and tadalafil (CIALIS®) are often prescribed for men with erectile dysfunction. Some of the ocular side effects include blue vision, and ischemic optic neuropathy. Blood Pressure Medications can cause glaucoma.

In some forms, the formulations and methods are used for treating, alleviating, and/or preventing one or more ocular symptoms that arise as a side effect from taking a systemic drug.

In some forms, the formulations and methods are used for treating, alleviating, and/or preventing one or more ocular symptoms in patients with ocular graft versus host disease. Ocular Graft Versus Host Disease (GVHD) occurs in patients who have undergone allogenic hematological stem cell transplantation. It can occur in patients who have acute or chronic GVHD, though it is more common in patients with the chronic form. Approximately 40-90% of patients with chronic GVHD will develop ocular symptoms.

Exemplary ocular manifestations include moderate to severe keratoconjunctivitis sicca, bilateral marginal keratitis, anterior uveitis, corneal ulceration or neovascularization. Thus, in some forms, the formulations are suitable for treating, alleviating, and/or preventing keratoconjunctivitis sicca, bilateral marginal keratitis, anterior uveitis, corneal ulceration or neovascularization. In one form, the formulations are used to improve vision of patients with ocular graft versus host disease after a bone marrow transplant for leukemia.

### C. Effective Amounts and Controls

The ophthalmic formulations can be administered to the eye therapeutically to achieve a therapeutic benefit, or prophylactically to achieve a prophylactic benefit, or to achieve both a therapeutic and prophylactic benefit. By therapeutic benefit is meant treating the underlying disorder including eradication or amelioration of one or more of the symptoms associated with the underlying disorder such that the patient reports an improvement in feeling or condition, notwithstanding that the patient can still be afflicted with the underlying disorder. For example, administration of a composition to a patient suffering from a condition provides therapeutic benefit not only when the underlying condition is eradicated or ameliorated, but also when the patient reports a decrease in the severity or duration of the symptoms associated with the condition. Therapeutic benefit also includes halting or slowing the progression of the disease, regardless of whether improvement is realized by the patient.

In preferred forms, the ophthalmic formulations are retained at the site of administration (e.g., at the surface of the cornea) for periods of time sufficient to yield a therapeutic effect. For example, when administered topically onto the cornea, the ophthalmic formulations penetrate the surface of the cornea to form a self-assembled structure that resists clearance by tearing, eye movement and diffusion at the site of the administration. In preferred forms, the amount of time that the ophthalmic formulations remain in contact with the surface of the cornea is sufficient to prevent or reduce corneal inflammation and edema. In certain forms, the ability to resist clearance from the site of administration and prevent or reduce corneal inflammation is associated with parameters such as the concentration and homogeneity of the ophthalmic formulations.

Typically, the ophthalmic formulations are administered to the eye in an effective amount in order to achieve a clinically significant result. Effective dosages and concentrations are those that provide a therapeutic benefit and can be determined according to the desired therapeutic or prophylactic result. For example, the ophthalmic formulations can be effective to treat and prevent one or more symptoms of an ocular disease or disorder. Exemplary symptoms that can be treated, prevented reduced or otherwise moderated by the ophthalmic compositions include, but are not limited to, pain, irritation, inflammation and swelling of the eye, redness or discoloration of the eye and/or surrounding tissues, dryness of the eye, discharge and/or watering of the eyes, impaired vision, blindness, photosensitivity, headache, fever, and combinations thereof.

In some forms, the ophthalmic formulations are administered in an amount effective to reduce or prevent inflammation of the eye. In some forms, the ophthalmic formulations are administered in an amount effective to reduce or prevent inflammation of the cornea or surface of the eye. Reducing inflammation can include reducing or preventing tissue damage, inducing or enhancing healing, reducing or preventing scaring, or combinations of these.

In some forms, the ophthalmic formulations are in an amount effective to provide a fluid impermeable barrier at the site of administration. The barrier structure is effective to prevent the movement of bodily fluids and contaminants through the structure. When the ophthalmic formulations provide a barrier structure at the surface of the eye, the presence of the barrier can be effective to treat diseases associated with recurrent infection or contamination of a damaged or missing corneal tissue. For example, in some forms, the presence of the ophthalmic formulations provides a continuous surface that prevents the formation of adhesions between the damaged corneal epithelium and other tissues. In other forms, the presence of the barrier structure prevents inflammatory processes responsible for the development and maintenance of eye diseases, such as chronic eye diseases (e.g., RCES).

In some forms, the ophthalmic formulations are in an amount effective to provide a fluid permeable structure at the site of administration. In some forms, the presence of a fluid permeable structure that acts as a membrane to facilitate passage of one or more fluids into and through the structure provides a suitable scaffold for the replacement, repair or augmentation of a diseased or damaged tissue on the surface of the eye or within the eye.

In further forms, the ophthalmic formulations is effective to provide an optically clear surface on which the structure and function of the tear film can be generated or restored.

These dosages and concentrations can guide the selection of dosages and concentrations in the present context. Bioactive molecules can be provided at a variety of suitable concentrations and in suitable amounts (e.g., in the microgram or milligram range, or greater). For guidance, one can consult texts such as Goodman and Gilman's The Pharmacological Basis of Therapeutics, 10th Ed., and Katzung, Basic and Clinical Pharmacology.

Inflammation and intraocular pressure (IOP) can be measured before and after administration of the compositions by methods known in the art. For example, a Reichert Tono-Pen contact tonometer can be used to assess the IOP, and changes in IOP, for example, in any eye compartment A subject, for example, a human patient, can be evaluated on several post-administration days, for example at 1, 7, 14 and 30 days post-administration, using the same evaluation procedure on each day. One or more measurements, for example about five, can be obtained for each subject at each time point. When measuring IOP according to methods known in the art, for example the method above, IOP is shown to be reduced by, for example, at least about 10%, or at least about 30%, or at least about 50% over what is observed when a control composition, for example a composition that does not include the ophthalmic compositions, is administered to the patient. To measure inflammation, slit-lamp bio-microscopy can be performed to examine any eye compartment, for example, the cornea, for signs of inflammation.

The examination can involve observation of criteria such as the presence of cells, flare and fibrin. The subjects can be evaluated on several post-injection days, for example at 1, 7, 14 and 30 days post-injection using the same evaluation procedure on each day. After general and/or local anesthesia is achieved, each subject can be examined for gross abnormalities. The exams can be performed by the same trained ophthalmologist, and the ophthalmologist can be blinded to the assignment of the treatment and control subjects.

Quantification of inflammation of an eye compartment such as the cornea can be performed using a modified version of the Standard Uveitis Nomenclature clinical grading scheme. When measuring inflammation according to methods known in the art, for example the method above, inflammation is shown to be reduced by, for example, at least about 10%, or at least about 30%, or at least about 50% over what is observed when an equivalent control composition, e.g., lacking the osmoprotective agent, is administered to the patient. When comparing an IOP or inflammation measurement obtained after administration of the ophthalmic formulations versus an IOP or inflammation measurement obtained for a control composition, the measurements compared can, and frequently should, be taken at similar time points post administration. For example, an IOP measurement after administration of the ophthalmic formulations taken 7 days post-administration is generally compared with an IOP measurement for a control composition taken 7 days post-administration.

In some forms, the ophthalmic formulations are effective to preserve and/or restore epithelial barrier integrity of the corneal surface. In some forms, epithelial barrier integrity is evaluated using transepithelial electrical resistance (TER), impedance-based measurements, or corneal permeability assays. In some forms, administration of the ophthalmic formulations results in an increase in TER by about 10% to about 70%, such as about 15% to about 60%, or about 20% to about 50%, in the subject compared to the same subject prior to administration or compared to a subject exposed to a preservative-containing composition in the absence of the ophthalmic formulations.

In some forms, the ophthalmic formulations are effective to preserve and/or restore expression and localization of epithelial adherens junction proteins. In some forms, such proteins include E-cadherin and β-catenin. In some forms, administration of the ophthalmic formulations results in an increase in expression levels of one or more adherens junction proteins by about 10% to about 80%, such as about 20% to about 70%, or about 30% to about 60% in the subject, compared to the same subject prior to administration or compared to a subject exposed to a preservative-containing composition in the absence of the ophthalmic formulations.

In some forms, the ophthalmic formulations are effective to reduce or prevent preservative-induced epithelial damage. Such damage can be assessed by fluorescein staining of the corneal surface, slit-lamp examination, or imaging-based scoring systems. For example, administration of the ophthalmic formulations results in a reduction in fluorescein staining or epithelial damage scores by about 10% to about 80%, such as about 20% to about 70%, or about 30% to about 60% in the subject, compared to the same subject prior to administration or compared to a subject exposed to a preservative-containing composition in the absence of the ophthalmic formulations.

In some forms, the ophthalmic formulations are effective to preserve corneal epithelial thickness and morphology. Epithelial thickness can be measured using histological analysis, optical coherence tomography (OCT), or confocal microscopy. For example, administration of the ophthalmic formulations reduces loss of epithelial thickness by about 5% to about 60%, such as about 10% to about 50%, or about 15% to about 40% in the subject, compared to the same subject prior to administration or compared to a subject exposed to a preservative-containing composition in the absence of the ophthalmic formulations.

In some forms, the ophthalmic formulations are effective to improve mitochondrial function in corneal epithelial cells. Mitochondrial function can be assessed by measuring ATP production, mitochondrial membrane potential, or oxygen consumption rates. For example, administration of the ophthalmic formulations results in an increase in ATP production by about 10% to about 100%, such as about 20% to about 80%, or about 30% to about 70% in the subject, compared to the same subject prior to administration or compared to a subject exposed to a preservative-containing composition in the absence of the ophthalmic formulations.

In some forms, the ophthalmic formulations are effective to modulate activity and/or expression of enzymes associated with the mitochondrial carnitine shuttle pathway. For example, such enzymes include but are not limited to CPT1, CPT2, and CrAT. In some forms, administration of the ophthalmic formulations results in an increase in expression or activity of one or more of these enzymes by about 10% to about 100%, such as about 20% to about 90%, or about 30% to about 80% in the subject, compared to the same subject prior to administration or compared to a subject exposed to a preservative-containing composition in the absence of the ophthalmic formulations.

### D. Combination Treatments

Methods of treating and preventing diseases and disorders of the eye including administering the ophthalmic formulations to the eye can include co-administration of one or more additional active agents, such as therapeutic, diagnostic or prophylactic agents. In some forms, the additional active agents include one or more classes of therapeutic agents for the treatment of ocular diseases, such as anti-inflammatory agents, vasoactive agents, anti-infective agents, anesthetics, growth factors, vitamins, nutrients, and/or cells that can be topically applied to the eyes. In some forms, the compositions include one or more ophthalmic drugs. Non-limiting examples of ophthalmic drugs include anti-glaucoma agents, anti-angiogenesis agents, anti-infective agents, anti-inflammatory agents, analgesics, anesthetics, growth factors, immunosuppressant agents, anti-allergic agents, anti-oxidants, cytokines, and combinations thereof.

In some forms, methods of treating and preventing eye diseases can include administering the ophthalmic formulations in combination with one or more anti-glaucoma agents, one or more anti-angiogenesis agents, one or more vasoconstricting agents, one or more anti-infective agents, one or more anti-inflammatory agents, or combinations.

Other therapeutic agents can be included by virtue of co-administration in the present methods. Examples include immuno-suppressants, vascular endothelial growth factor (VEGF), a transforming growth factor (TGF) such as transforming growth factor p, a platelet derived growth factor (PDGF), an epidermal growth factor (EGF), a nerve growth factor (NGF), an insulin-like growth factor (e.g., insulin-like growth factor I), a glial growth factor (GGF), a fibroblast growth factor (FGF), etc. It will be appreciated that in many cases these terms refer to a variety of different molecular species. For example, several transforming growth factor R species are known in the art. One of ordinary skill in the art will be guided in the selection of an appropriate growth factor by considering, for example, the disease or disorder which is to be treated.

The growth factor or another agent can be a chemotactic substance, which has the ability, in vivo or in cell culture, to recruit cells to a site at which the substance is present. The recruited cells may have the potential to contribute to the formation of new tissue or to augment and reinforce existing, damaged tissue (e.g., by contributing structurally and/or functionally to the tissue (e.g., by providing growth factors or contributing to a desirable immune response). Certain chemotactic substances can also function as proliferation agents (e.g., neurotropic factors such as NGF or BDNF).

While in the foregoing specification the compositions and methods have been described in relation to certain forms thereof, and many details have been put forth for the purpose of illustration, it will be apparent to those skilled in the art that additional embodiments, and certain of the details described herein can be varied considerably without departing from the basic principles set forth above.

The ophthalmic compositions and methods can be further understood by one or more of the following numbered paragraphs.
Paragraph 1. An ophthalmic composition including:
   (i) one or more preservatives that causes hyperosmolarity in an amount effective to prevent or reduce microbial contamination, and
   (ii) one or more osmoprotective agents in an amount effective to prevent or reduce preservative-induced hyperosmolarity and/or ocular surface damage.
Paragraph 2. The ophthalmic composition of paragraph 1, wherein
   the one or more osmoprotective agents are selected from L-carnitine, L-carnitine derivative or analog thereof, wherein the L-carnitine derivative or analog is selected from the group of acetylcarnitine (C2:0), propionylcarnitine (C3:0), butyrylcarnitine (C4:0), and valerylcarnitine (C5:0), and combinations thereof and/or
   the one or more preservatives is selected from an quaternary ammonium optionally benzalkonium chloride (BAC), polyquaternium-1, polyquaternium-42, or cetrimide; an oxidizing agent optionally sodium perborate or stabilized oxochloro complex; an ionic buffer optionally borate, sorbital, propylene glycol, or zing; an amidine optionally chlorhexidine; a paraben optionally methylparaben or propylparaben; an alcohol optionally chlorobutanol or phenylethanol; a mercury-based chemical optionally thimerosal or phenylmercuric nitrate/acetate; and combinations thereof.
Paragraph 3. The ophthalmic composition of paragraph 1 or 2, wherein preservative is present in the composition in a concentration ranging from 0.001% wt/v to 0.02% wt/v.
Paragraph 4. The ophthalmic composition of any one of paragraphs 1-3, wherein the one or more osmoprotective agents are present in the composition in a concentration ranging from 0.1% wt/v to 5.0% wt/v.
Paragraph 5. The ophthalmic composition of any one of paragraphs 1-4, further including a pharmaceutically acceptable carrier suitable for ocular administration.
Paragraph 6. The ophthalmic composition of paragraph 5, wherein the pharmaceutically acceptable carrier includes an aqueous buffer solution with a pH between 6.5 and 7.8.
Paragraph 7. The ophthalmic composition of any one of paragraphs 1-6, further including one or more additional agents selected from the group of viscosity-enhancing agents; chelating agents; pH-adjusting agents; stabilizers; buffering agents, lubricants, tonicity adjusting agents, demulcents, and preservatives.
Paragraph 8. The ophthalmic composition of any one of paragraphs 1-7, further including one or more additional active agents selected from the group of anti-glaucoma agents, anti-inflammatory agents, antihistamines or mast cell stabilizers, antibiotics or antiviral agents.
Paragraph 9. The ophthalmic composition of any one of paragraphs 1-8, wherein the ophthalmic composition is formulated as an aqueous solution, suspension, gel, ointment, or emulsion suitable for ocular administration.
Paragraph 10. The ophthalmic composition of any one of paragraphs 1-9, wherein the ophthalmic composition is incorporated into an ocular delivery system selected from the group of ocular wound bandages, ocular inserts, ocular gel drops, contact lenses, contact lens solution, and periocular patches.
Paragraph 11. The ophthalmic composition of paragraph 1, wherein the composition is formulated as artificial tears, an anti-glaucoma medication, an ophthalmic lubricant, an anti-allergy eye drop, an anti-inflammatory eye drop, a post-operative ophthalmic treatment, an ocular wound healing aid, an antimicrobial ophthalmic preparation, a mydriatic eye drop, cycloplegic eye drops, a prophylactic or therapeutic product for treating or managing ocular surface or anterior segment conditions, or a combination thereof.
Paragraph 12. The ophthalmic composition of paragraph any one of paragraphs 1-11, having an osmolality ranging from 150 mOsm/kg to 500 mOsm/kg, optionally wherein the osmolality ranges from 280 mOsm/kg to 320 mOsm/kg.
Paragraph 13. A method of reducing or preventing preservative-induced ocular damage, including administering to one or both eyes of a subject in need thereof, the ophthalmic composition of any one of paragraphs 1-12.
Paragraph 14. A method of reducing or preventing preservative-induced hyperosmolarity in a subject in need thereof, including administering to one or both eyes of a subject in need thereof, the ophthalmic composition of any one of paragraphs 1-12.
Paragraph 15. The method of paragraph 13 or paragraph 14, wherein the composition is administered to a subject undergoing topical ophthalmic therapy requiring medication.
Paragraph 16. The method of any one of paragraphs 13-15, wherein the ophthalmic composition is administered to treat, prevent, or as part of a therapeutic regimen for an condition associated with ocular surface hyperosmolarity, wherein the condition is selected from the group of dry eye disease, keratoconjunctivitis sicca, meibomian gland dysfunction, allergic conjunctivitis, blepharitis, corneal epithelial defects, superficial punctate keratitis, conjunctival inflammation, corneal abrasion, ocular graft-versus-host disease, post-surgical ocular surface inflammation, contact lens-related discomfort, post-operative ocular irritation, environmentally induced dry eye, or any combination thereof.
Paragraph 17. The method of any one of paragraphs 13-16, wherein the composition reduces one or more markers of ocular surface toxicity selected from the group of epithelial cell apoptosis, adherens junction disruption, tear film instability, corneal staining, and inflammation, in the subject, compared to the same subject prior to administration or a subject not administered the composition or the subject administered the same composition without the osmoprotective agent.
Paragraph 18. The method of any one of paragraphs 13-16, wherein the subject is selected from the group of patients with ocular surface disorders, individuals undergoing long-term topical ophthalmic therapy, post-operative patients recovering from refractive or cataract surgery, elderly individuals with age-related tear film instability, contact lens wearers, and patients with autoimmune conditions affecting the eye (such as Sjögren's syndrome or ocular graft-versus-host disease), or a combination thereof.
Paragraph 19. The method of any one of paragraphs 13-18, wherein the ophthalmic composition is administered topically to the ocular surface one to six times daily.
Paragraph 20. The method of any one of paragraphs 13-19, wherein the ophthalmic composition is administered as part of an acute or chronic treatment regimen tailored to the severity of the ocular surface condition.
Paragraph 21. A composition or method according to any of the text, figures, or combination thereof provided herein.
Paragraph 22. The method of paragraph 13 or paragraph 14, wherein administering the ophthalmic composition to the subject increases expression of one or more enzymes selected from CPT1, CPT2, and CrAT in corneal epithelial cells of the subject, compared to the same subject prior to administration of the ophthalmic composition or a different subject not administered the ophthalmic composition.
Paragraph 23. The method of paragraph 13 or paragraph 14, wherein administering the ophthalmic composition to the subject increases transepithelial electrical resistance (TER) in corneal epithelial cells of the subject, compared to the same subject prior to administration of the ophthalmic composition or a different subject not administered the ophthalmic composition.

All references cited herein are incorporated by reference in their entirety. The present description will be further understood by reference to the following non-limiting examples.

### EXAMPLES

### Example 1: In-Vitro Studies Investigating the Effects of L-Carnitine on Benzalkonium Chloride-Induced Ocular Surface Toxicity

### Materials and Methods

### Cell Culture and Treatment Conditions

Telomerase-immortalized human corneal epithelial cells (hTCEpi) were cultured in keratinocyte serum-free medium (KSFM) supplemented with bovine pituitary extract and recombinant epidermal growth factor. Cells were maintained under standard conditions (37 °C, 5% CO₂) and seeded into appropriate culture vessels until reaching confluence prior to experimentation.

To induce hyperosmolar stress, culture medium was supplemented with sodium chloride (NaCl) to final concentrations of 70, 90, or 120 mM, corresponding to approximately 450, 500, and 550 mOsm, respectively. For benzalkonium chloride (BAC) exposure studies, cells were treated with 0.002%, 0.02%, or 0.2% BAC in serum-free media for either 6 or 24 hours. Untreated (UT) cells served as controls for all experiments.

### Transepithelial Electrical Resistance (TER) Measurement

Epithelial barrier integrity was assessed by measuring TER using an ECIS z-theta system. For NaCl and BAC treatments, TER was recorded over a 24-hour period and normalized to baseline values (pre-treatment). For wound healing assays, TER was continuously monitored for up to 72 hours post-injury using an electric cell-substrate impedance sensing (ECIS) system. The timepoint of treatment or wounding was marked as 0 hours. Each condition included a minimum of four independent biological replicates.

### Electrical Wounding and TER-Based Wound Healing Assay

Wound healing was assessed using the ECIS system. Confluent monolayers of hTCEpi cells were wounded electrically by applying a 5,000 μA current for 30 seconds at a frequency of 4000 Hz. TER recovery was monitored in real time post-wounding to assess epithelial migration and barrier restoration. Migration time (T) and migration rate (V) were calculated from TER recovery profiles.

### L-Carnitine Supplementation

In rescue experiments, 200 mM L-carnitine (LCAR) was added to the culture medium 1 hour prior to NaCl treatment and maintained throughout the experimental duration. Experimental groups included UT, NaCl-only, and NaCl+LCAR conditions.

### Gene Expression Analysis

Total RNA was extracted using a commercial RNA isolation kit. First-strand cDNA was synthesized using reverse transcriptase, and quantitative PCR (qPCR) was performed using gene-specific primers. For adherens junction protein analysis, primers targeting E-cadherin, β-catenin, α-catenin, and p120-catenin were used. For Wnt signaling pathway analysis, primers for WNT1, LRP6, DKK1, and WIF1 were utilized. GAPDH served as the internal reference gene. Relative expression levels were determined using the ΔΔCt method. Each experimental condition included 4-33 biological replicates depending on the assay.

### Western Blot Analysis

For protein expression analysis, cells were lysed and total protein was quantified using a BCA assay. Equal protein amounts were separated by SDS-PAGE and transferred onto PVDF membranes. Membranes were probed with antibodies specific to E-cadherin, β-catenin, α-catenin, p120-catenin, and β-actin (used as a loading control). Protein bands were visualized using chemiluminescence and quantified via densitometry. Results were normalized to β-actin levels.

### Immunofluorescence Staining and Microscopy

Cells were fixed in 4% paraformaldehyde, permeabilized, and incubated with antibodies targeting E-cadherin (red) or β-catenin (green). Nuclei were counterstained with DAPI. Images were captured using confocal microscopy. Quantitative image analysis was conducted across 8-10 randomly selected fields per group, and relative fluorescence intensity (RFI) was normalized to total cell count.

### Statistical Analysis

Data are presented as mean±standard error of the mean (SEM). Statistical significance was assessed using one-way ANOVA followed by appropriate post hoc tests. p-values less than 0.05 were considered statistically significant, and significance levels were denoted as follows: p<0.05, *p<0.01, **p<0.001, and ***p<0.0001.

### Results

### Hyperosmolarity Compromises Epithelial Barrier Function and Junctional Protein Expression in a Dose-Dependent Manner

Hyperosmotic NaCl treatment reduces TER in a dose-dependent manner hTCEpi cells treated with increasing concentrations of NaCl (70, 90, and 120 mM) exhibited a progressive, dose-dependent decline in epithelial barrier function as measured by normalized TER over 24 hours. Compared to untreated controls, all NaCl-treated groups showed significantly reduced TER (p<0.0001), with the most pronounced effects observed in the 120 mM NaCl group (FIG. 1).

### E-Cadherin Expression Decreases Under Hyperosmotic Stress

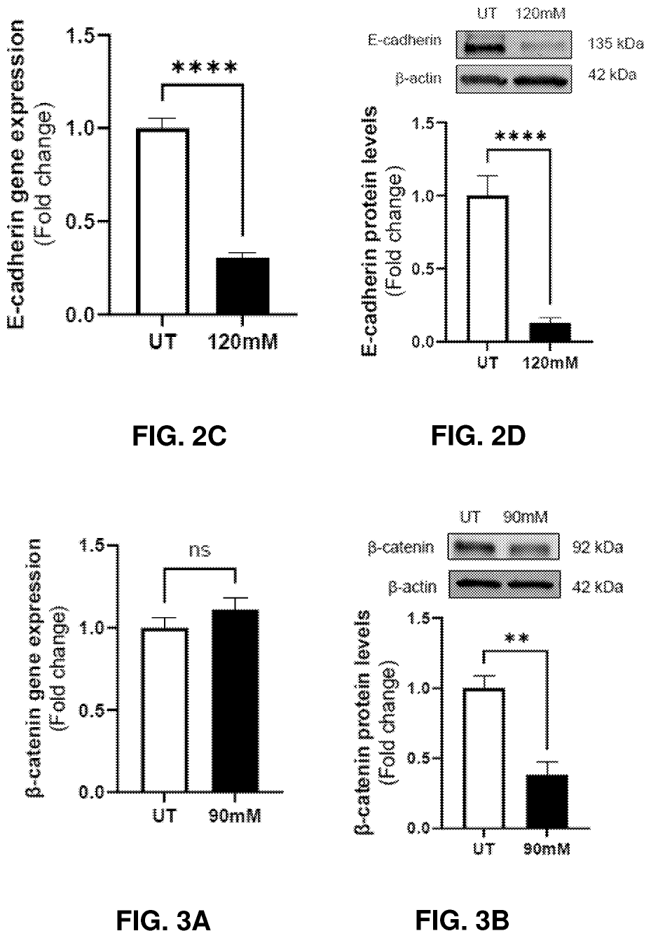

Exposure to 90 mM and 120 mM NaCl for 24 hours significantly reduced both mRNA and protein levels of E-cadherin compared to untreated cells (FIG. 2). These findings suggest a disruption in epithelial cell-cell adhesion under hyperosmolar conditions.

### β-Catenin Levels are Downregulated in Response to NaCl Treatment

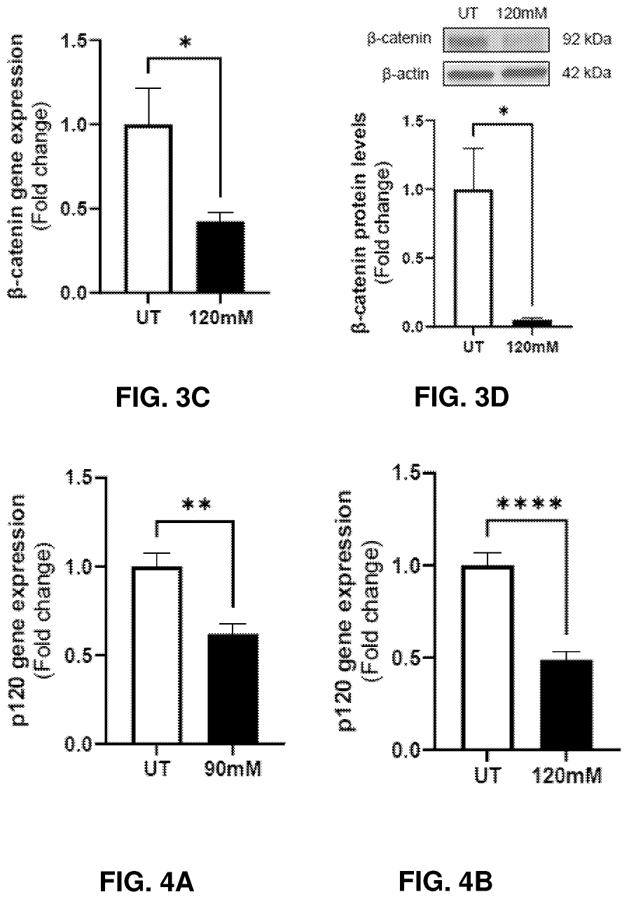

Protein levels of β-catenin were significantly decreased in both 90 mM and 120 mM NaCl groups, while mRNA levels remained unchanged at 90 mM but were significantly reduced at 120 mM (FIG. 3). This indicates both transcriptional and post-transcriptional regulation under severe osmotic stress.

### β-Catenin and p120-Catenin are Differentially Affected by Osmolarity

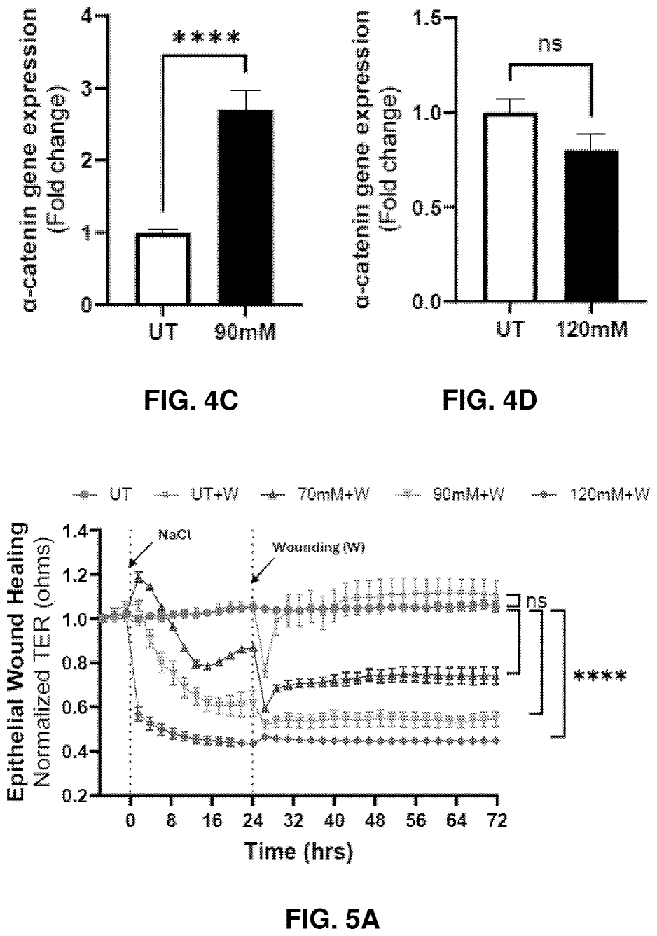

Gene expression of p120-catenin was significantly reduced in both 90 mM and 120 mM NaCl-treated cells. In contrast, α-catenin expression was significantly elevated at 90 mM NaCl but remained unchanged at 120 mM NaCl (FIG. 4), suggesting a compensatory or stress-induced regulatory mechanism at moderate osmolarity.

### Hyperosmolar Stress Impairs Corneal Epithelial Wound Healing

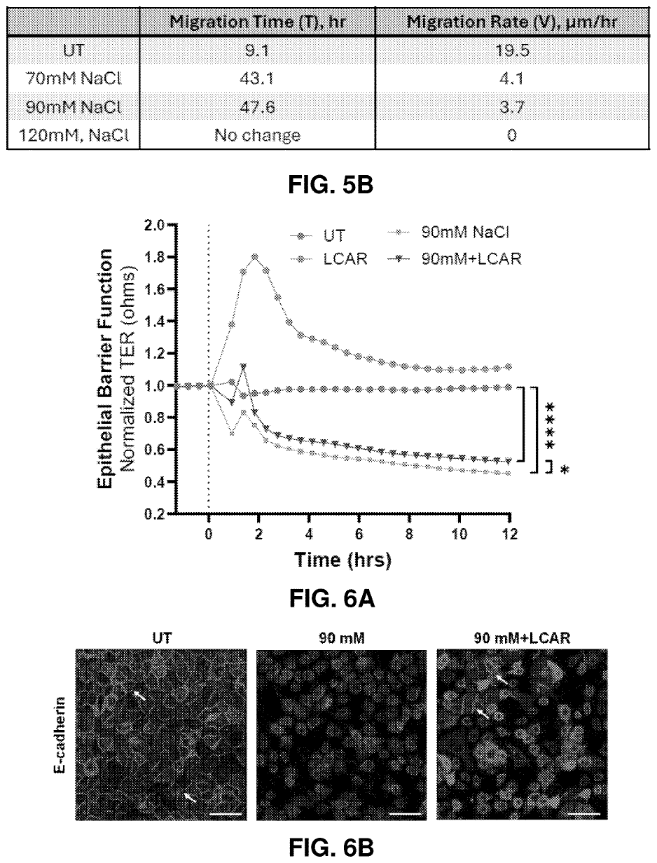

Wound healing, assessed by normalized TER following electrical disruption of the monolayer, was significantly impaired in a dose-dependent manner under hyperosmotic conditions. hTCEpi cells treated with 70, 90, and 120 mM NaCl exhibited delayed TER recovery after wounding, with 120 mM NaCl completely inhibiting wound closure (FIG. 5A). Quantitative analysis showed a marked increase in migration time and a significant reduction in migration rate compared to untreated cells, with no detectable migration in the 120 mM NaCl group (FIG. 5B).

### L-Carnitine Restores Barrier Function and Adherens Junction Protein Expression

L-carnitine (LCAR) supplementation at 200 mM significantly improved epithelial barrier function in hTCEpi cells exposed to 90 mM NaCl, as evidenced by improved TER values compared to NaCl-only treatment (FIG. 6A). Immunofluorescence staining revealed reduced E-cadherin and β-catenin localization under hyperosmolar stress, which was restored upon L-carnitine co-treatment (FIGS. 6B, 6E). Quantitative analysis confirmed significant recovery of both E-cadherin and β-catenin levels at the protein (FIGS. 6C, 6F) and transcript level (FIGS. 6D, 6G), approaching levels observed in untreated controls.

### L-Carnitine Normalizes Wnt/β-Catenin Pathway Gene Expression

Hyperosmolarity reduced the expression of canonical Wnt signaling activators WNT1 and LRP6 (FIGS. 7A, 7B), while increasing expression of pathway inhibitors DKK1 and WIF1 (FIGS. 7C, 7D). L-carnitine supplementation reversed these changes, restoring WNT1 and LRP6 levels while significantly reducing DKK1 and WIF1 expression. These results suggest that L-carnitine mitigates hyperosmolar stress-induced barrier dysfunction, in part by rebalancing Wnt/β-catenin signaling.

### BAC Exposure Impairs Epithelial Barrier Function and Alters Adherens Junction Gene Expression

### BAC Compromises Epithelial Barrier Integrity in a Dose-Dependent Manner

Treatment of hTCEpi cells with benzalkonium chloride (BAC) at concentrations of 0.002%, 0.02%, and 0.2% for 24 hours resulted in a significant and dose-dependent decline in transepithelial electrical resistance (TER), indicating impaired barrier function (FIG. 9). All BAC-treated groups showed a significant reduction in TER compared to untreated controls, with the 0.2% group exhibiting the most profound effect (***p<0.0001).

### Short-Term BAC Exposure Reduces E-Cadherin and β-Catenin Expression

After 6 hours of treatment with 0.002% BAC, hTCEpi cells exhibited significantly decreased E-cadherin gene expression and moderately increased β-catenin gene expression compared to untreated controls (FIG. 11A-B). These results suggest an early disruption in adherens junction regulation.

### Prolonged BAC Exposure Exacerbates Junctional Protein Gene Downregulation

At 24 hours post-treatment with 0.002% BAC, gene expression of both E-cadherin and β-catenin was markedly suppressed compared to untreated cells (FIG. 10A-B). E-cadherin levels dropped nearly completely, while β-catenin expression was significantly diminished (*p<0.01). These findings indicate a time-dependent, deleterious effect of BAC on epithelial junctional gene expression.

### Example 2: In-Vivo Studies Demonstrating that L-Carnitine Alleviates BAC-Induced Ocular Surface Damage in Mice

### Materials and Methods

### Animals and Housing

Male and female C57BL/6 mice (6-8 weeks old) were used for all experiments. Mice were housed under standard laboratory conditions (12-hour light/dark cycle, 22±2 °C, 50±10% humidity) with ad libitum access to food and water. All procedures were approved by the Institutional Animal Care and Use Committee (IACUC) and adhered to the ARVO Statement for the Use of Animals in Ophthalmic and Vision Research.

### Topical Treatment Regimen

To induce ocular surface damage, 0.2% benzalkonium chloride (BAC) solution was administered topically to both eyes twice daily (10 μL/eye) for 5-7 consecutive days. For rescue experiments, 200 mM L-carnitine (LCAR) was co-administered with BAC in the same volume and frequency. Treatment groups included: vehicle control (PBS), BAC alone, BAC+LCAR, and LCAR alone.

### Slit Lamp Microscopy and Fluorescein Staining

Corneal surface integrity was assessed using slit lamp microscopy with and without fluorescein dye. For epithelial damage evaluation, 1 μL of 0.25% fluorescein was applied to each eye, and images were captured under cobalt blue light. Corneal opacity was assessed from white light images. Neovascularization and corneal surface irregularities were noted qualitatively. Images were obtained and analyzed using standardized protocols by masked observers.

### Tear Film Measurement

Tear volume was quantified using phenol red thread testing. A phenol red-impregnated cotton thread was placed in the lateral canthus of each eye for 15 seconds. The length of the wetted (red) portion was immediately measured in millimeters. Measurements were taken on the final day of treatment. Data were collected for both sexes and analyzed separately and in combination.

### Corneal Opacity and Damage Scoring

Corneal opacity and epithelial damage were scored by masked observers on standardized 0-10 point scales based on slit lamp images. Opacity scoring considered haze density and area involved, while epithelial damage scoring incorporated fluorescein staining intensity and spread. Scores were averaged per eye.

### Histological Analysis

At endpoint, mice were euthanized, and eyes were enucleated, and embedded in frozen OCT molds. Sections (5 μm) were stained with hematoxylin and eosin (H & E) for evaluation of overall morphology. Images were captured at 2× and 20× magnification to assess corneal structure and epithelial integrity.

### Measurement of Corneal Thickness

Cross-sectional H & E-stained images were used to quantify total corneal and epithelial thickness using calibrated image analysis software (e.g., ImageJ). Thickness measurements were obtained from the central cornea in at least 3 sections per eye and averaged per animal.

### Immunofluorescence Staining of Corneal Sections

Formalin-fixed paraffin-embedded sections were deparaffinized, rehydrated, and subjected to antigen retrieval. Sections were incubated overnight at 4 °C with primary antibodies against E-cadherin (green) or β-catenin (red), followed by appropriate fluorophore-conjugated secondary antibodies. Nuclei were counterstained with DAPI (blue). Images were acquired at 20× magnification, and representative sections were shown. Scale bars represent 50 μm.

### Statistical Analysis

All data are presented as mean±SEM. One-way ANOVA with Tukey's post hoc test was used to evaluate differences among groups. Comparisons were considered statistically significant at p<0.05. Group sizes are specified in figure legends and ranged from 8-37 eyes per group depending on the assay.

### Results

### Slit Lamp Imaging Reveals Protection Against Corneal Opacity and Damage

Topical administration of 0.2% benzalkonium chloride (BAC) for 5-7 days induced significant corneal abnormalities in both male and female mice, including increased opacity, fluorescein-stained epithelial defects, and neovascularization (data not shown). Co-treatment with 200 mM L-carnitine (BAC+LCAR) visibly reduced these pathological changes, resulting in clearer corneas and decreased staining, similar to vehicle-treated controls. L-carnitine alone did not induce any visible ocular damage.

### Tear Film Stability is Compromised by BAC and Partially Restored by L-Carnitine

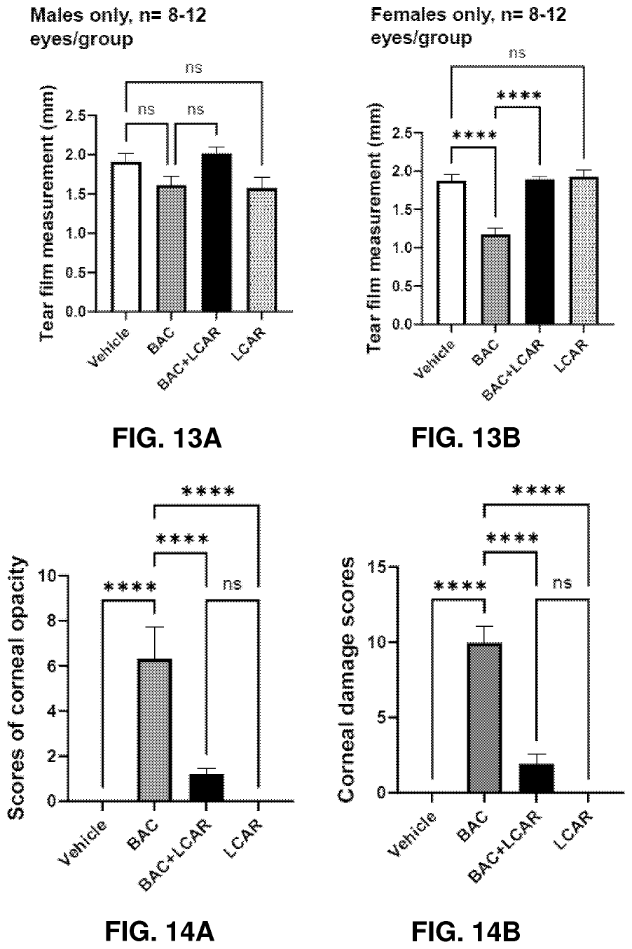

Phenol red thread testing demonstrated significantly reduced tear film production in BAC-treated mice compared to vehicle controls (**p<0.001). Co-treatment with L-carnitine restored tear volume to levels comparable with controls (***p<0.0001), while L-carnitine alone had no significant effect on tear production (FIG. 12). Stratified analysis by sex revealed that BAC significantly reduced tear production in female mice but not in males. L-carnitine co-treatment reversed this effect in females (***p<0.0001), with no significant differences observed across male groups (FIGS. 13A and 13B).

### L-Carnitine Prevents BAC-Induced Corneal Opacity and Epithelial Damage

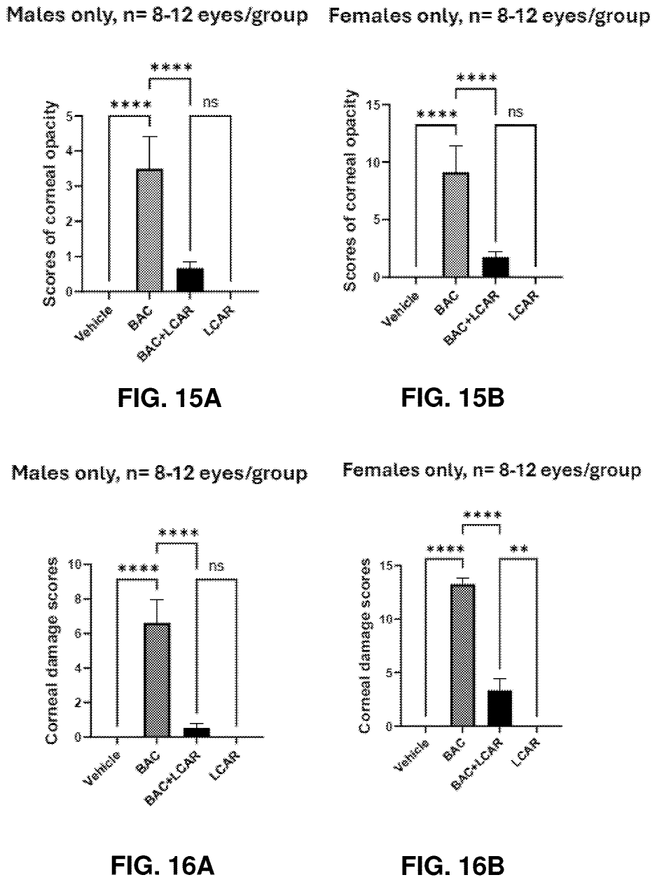

Quantitative scoring of corneal opacity and damage revealed significant injury in BAC-treated mice relative to vehicle controls (***p<0.0001). Co-treatment with L-carnitine significantly reduced both opacity and damage scores, restoring values near baseline (***p<0.0001), while L-carnitine alone did not affect baseline scores (FIGS. 14A and 14B). Sex-specific analyses further confirmed this protective effect in both males and females (FIGS. 15A and 15B and FIGS. 16A and 16B respectively).

### Corneal Morphology is Preserved by L-Carnitine Treatment

Histological examination of H & E-stained corneal sections at 2× and 20× magnification revealed marked epithelial thinning and structural disorganization in BAC-treated eyes. In contrast, co-treatment with L-carnitine preserved epithelial integrity and overall morphology, comparable to vehicle and L-carnitine-only groups (data not shown).

### L-Carnitine Rescues BAC-Induced Loss of Corneal Epithelium Thickness

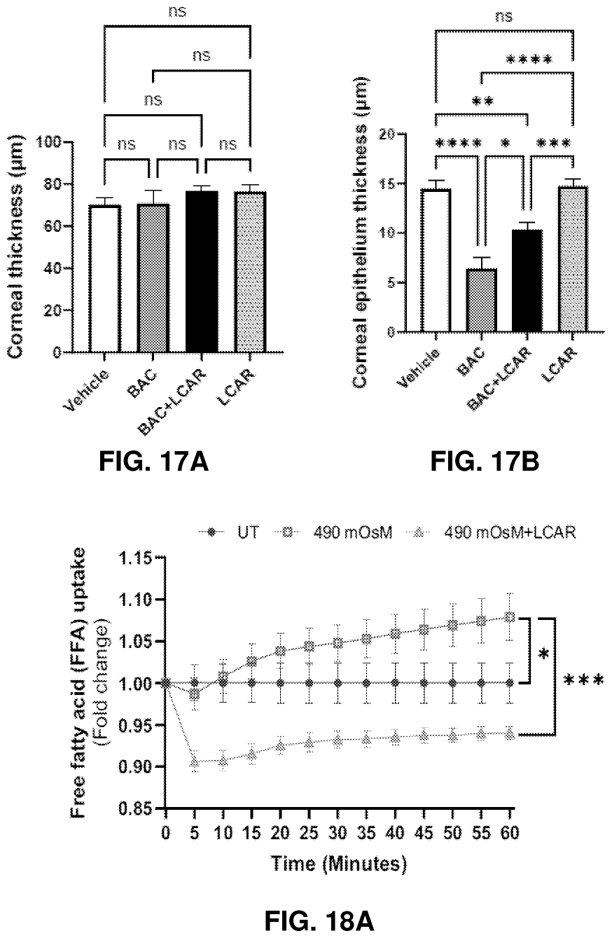

Although total corneal thickness remained unchanged across all treatment groups, BAC significantly reduced epithelial thickness compared to vehicle controls (***p<0.0001). L-carnitine supplementation restored epithelial thickness to near-normal levels (**p<0.001), while no significant difference was observed in the L-carnitine-only group (FIGS. 17A and 17B).

### Immunostaining Confirms L-Carnitine Restores Junctional Protein Expression

Immunofluorescence analysis of β-catenin and E-cadherin in corneal tissue sections revealed substantial loss of signal in BAC-treated mice. Co-treatment with L-carnitine markedly restored expression of both proteins within the corneal epithelium, indicating preservation of adherens junction integrity. L-carnitine alone did not alter junctional protein expression in comparison to vehicle controls (data not shown).

The present findings demonstrate that topical co-administration of L-carnitine with BAC markedly protects the cornea against benzalkonium chloride (BAC)-induced toxicity. L-carnitine prevented corneal opacity and epithelial thinning, maintained normal epithelial morphology, and preserved cell-cell junction proteins (E-cadherin and β-catenin). L-carnitine and its derivatives are naturally present in tears and are significantly depleted in dry eye patients (Pescosolido N, et al. L-carnitine and short chain ester in tears from patients with dry eye. Optom Vis Sci. 2009 February; 86(2):E132-8, Cong Y, et al., Recommendations for nutritional supplements for dry eye disease: current advances. Front Pharmacol. 2024 May 30; 15:1388787). The present findings of preservation of epithelial junctional proteins (E-cadherin and β-catenin) indicate that L-carnitine helps maintain epithelial barrier integrity.

Several mechanisms may underlie L-carnitine's protective actions. First, osmoprotection is central: L-carnitine is a compatible organic osmolyte that cells accumulate to balance extracellular hyperosmolarity without perturbing metabolism (PMID: Wolffsohn J S, et al., Clinical practice patterns in the management of dry eye disease: A TFOS international survey 2023-24. Ocul Surf 2025 April; 36:164-172). By restoring cell volume and ionic balance, L-carnitine likely counteracts BAC's tendency to dehydrate and shrink epithelial cells (similar to dry eye stress). This osmotic buffering effect can help stabilize the tear film and prevent tear-breakup, consistent with the present observation of improved tear film stability.

Second, antioxidant effects of L-carnitine can potentially contribute to reducing BAC-induced ocular surface damage. BAC and hyperosmolar conditions induce oxidative stress in corneal cells, leading to lipid peroxidation and DNA damage. Suppression of reactive oxygen species (ROS) in human corneal epithelial cells can help restore and/or maintain physiological levels of antioxidant enzymes. In the present study, this antioxidant action would reduce membrane lipid peroxidation and mitochondrial DNA damage, preserving cell viability. Thus, by attenuating oxidative injury, L-carnitine likely preserves epithelial structure and prevents BAC-induced opacity.

Third, anti-inflammatory signaling can also potentially contribute to reduced ocular surface damage following BAC exposure. BAC exposure and dry eye triggers robust inflammatory cytokine release, which can disrupt epithelial junctions. In the present study, it was observed that L-carnitine preserves E-cadherin/β-catenin, which suggests lower inflammatory-mediated junctional breakdown. L-carnitine strongly suppresses hyperosmolarity-induced pro-inflammatory cytokines in vitro. For example, L-carnitine reduced TNF-α, IL-1β, IL-6, IL-8 and chemokines by 50-80% in stressed corneal epithelial cells (Hua X, et al., Effects of L-carnitine, erythritol and betaine on pro-inflammatory markers in primary human corneal epithelial cells exposed to hyperosmotic stress. Curr Eye Res. 2015 July; 40(7):657-67). This anti-inflammatory effect occurs in part via inhibition of the TRPV1-calcium pathway. By blocking TRPV1-mediated calcium influx, L-carnitine dampens osmotic-stress signaling and NF-κB activation, thereby lowering downstream MAPK and COX-2 activity (Hua X, et al., Effects of L-carnitine, erythritol and betaine on pro-inflammatory markers in primary human corneal epithelial cells exposed to hyperosmotic stress. Curr Eye Res. 2015 July; 40(7):657-67; Harrell C R, et al., “The Molecular Mechanisms Responsible for Tear Hyperosmolarity-Induced Pathological Changes in the Eyes of Dry Eye Disease Patients.” Cells. 2023 Dec. 1; 12(23):2755; Cong Y, et al., Recommendations for nutritional supplements for dry eye disease: current advances. Front Pharmacol. 2024 May 30; 15:1388787. Prior studies report that L-carnitine suppresses stress-activated kinases JNK/p38 MAPK and matrix metalloproteinases under hyperosmotic conditions (Hua X, et al., Effects of L-carnitine, erythritol and betaine on pro-inflammatory markers in primary human corneal epithelial cells exposed to hyperosmotic stress. Curr Eye Res. 2015 July; 40(7):657-67). Taken together, L-carnitine's inflammation-modulating actions can potentially prevent BAC from eliciting corneal inflammation and cell death, evidenced by observation in the present study of less epithelial damage and inflammatory marker expression following L-carnitine supplementation.

Finally, membrane and metabolic stabilization can possibly play a role in reducing BAC-induced ocular damage. L-carnitine is involved in mitochondrial fatty acid transport and energy metabolism. By increasing mitochondrial function and ATP production, L-carnitine can help cells recover from BAC-induced metabolic stress. L-carnitine has been shown to improve membrane integrity: in ocular hypertension models it reduced retinal cell apoptosis and markedly lowered lipid peroxidation (Calandrella N, et al., Carnitine reduces the lipoperoxidative damage of the membrane and apoptosis after induction of cell stress in experimental glaucoma. Cell Death Dis. 2010 Aug. 5; 1(8): e62). In the present study, preservation of cell-cell junctions indicates that epithelial membranes remained intact; L-carnitine's membrane-protective properties could underlie this effect.

In conclusion, topical L-carnitine robustly protects against BAC-induced corneal damage in mice, preserving epithelial thickness, morphology, and junctional protein expression without causing any toxicity on its own. By stabilizing cell volume, scavenging reactive oxygen species, and dampening inflammatory signaling, L-carnitine helps maintain tear film stability and epithelial integrity under stress. Clinically, L-carnitine's excellent safety profile and multi-modal activity make it an attractive candidate for inclusion in treatments for dry eye disease, especially in cases complicated by BAC or other chemical insults.

### Example 3: Altered Carnitine Homeostasis Modulates Hyperosmolarity-Induced Mitochondrial Dysfunction and Lipotoxicity in Human Corneal Epithelial Cells

### Materials and Methods

### Cell Culture

Human telomerase-immortalized corneal epithelial (hTCEpi) cells²² were cultured in complete Keratinocyte Growth Medium-2 (KGM-2; C-20111; PromoCell, Heidelberg, Germany), (as describe herein and ¹⁰). Prior to treatment, cells were maintained in basal KGM-2 medium without supplements. Hyperosmolar stress was induced by adding 90 mM NaCl for 6 hours, resulting in a final osmolarity of 490 mOsM. L-carnitine (200 mM; 8400920025; MilliporeSigma, Burlington, MA, USA) was administered either as a 2-hour pre-treatment or concurrently with NaCl for all experiments.

### Measurement of Free Fatty Acid Uptake

The hTCEpi cells were seeded in 96-well plates, allowed to adhere overnight, and treated as described above. Free fatty acid (FFA) uptake was assessed using a commercially available kinetic assay kit (ab176768; Abcam, Cambridge, UK) according to the manufacturer's instructions. Fluorescence intensity was measured at 485 nm excitation and 515 nm emission using a microplate reader (Synergy H1; Agilent Technologies, Inc., Santa Clara, CA, USA). Readings were recorded at 5-minute intervals for 60 minutes, and data were normalized as fold change relative to the mean fluorescence intensity of untreated cells.

### Lipid Extraction and Triglyceride Measurement

Lipid extraction from hTCEpi cells was performed using a modified Folch method.²³ Cells were seeded in 12-well plates, treated as described, and pooled (4 wells per sample). A total of 100 μL of chloroform:methanol (2:1, v/v) was added to each sample, followed by centrifugation at 14,000×g for 10 minutes to achieve phase separation. The aqueous phase was discarded, the interphase was collected for protein normalization, and the organic phase containing lipids was transferred to glass vials and evaporated overnight. Total triglyceride (TG) content was quantified using a colorimetric assay kit (10010303; Cayman Chemical, Ann Arbor, MI, USA) according to the manufacturer's instructions, with absorbance measured at 530 nm. Data were expressed as mg/g protein.

### Nile Red Staining for Lipid Accumulation

Lipid droplet accumulation in hTCEpi cells under hyperosmolar stress was assessed using Nile Red staining (N1142; Thermo Fisher Scientific, Waltham, MA, USA). Cells were seeded in 96-well plates and allowed to adhere overnight. Following treatment, cells were incubated with Nile Red (1 μg/mL) for 30 minutes at room temperature, washed with PBS, and counterstained with Hoechst 33342 to visualize nuclei. For each well, 3 to 6 fields were imaged at 20× magnification using a BZ-X710 fluorescence microscope (Keyence, Itasca, IL, USA). Fluorescence intensity of the lipid droplets was quantified using ImageJ software (NIH, Bethesda, MD, USA), and results were expressed as relative fluorescence intensity (RFI) normalized to the number of nuclei.

### Malondialdehyde Assay

Lipid peroxidation was quantified by measuring malondialdehyde (MDA) levels using a colorimetric assay kit (ab233471; Abcam). Following treatment, hTCEpi cells were lysed in RIPA buffer, centrifuged at 12,000×g for 10 minutes at 4 °C, and the supernatants were diluted 1:2 in assay buffer. Samples and MDA standards were added to a 96-well plate, followed by addition of MDA color reagent. Absorbance was measured at 695 nm using a Synergy H1 microplate reader, and MDA levels (μM) were calculated using the standard curve.

### Determination of Mitochondrial Superoxide Generation

Mitochondrial superoxide production was assessed using MitoSOX Red (Thermo Fisher Scientific), a fluorogenic dye selective for the detection of superoxide in the mitochondria of live cells. The hTCEpi cells were seeded in 96-well plates and treated as described above. Following treatment, the cells were washed with PBS and incubated with 1 μM MitoSOX Red for 10 minutes at 37 °C in the dark. For each well, 4 fields were imaged at 20× magnification using a BZ-X710 fluorescence microscope (Keyence). Fluorescence intensity was quantified using ImageJ software and expressed as RFI per field of view.

### Determination of Cellular ATP Levels

Intracellular ATP levels were measured using a fluorometric ATP assay kit (ab83355; Abcam). Following treatment, cells were lysed in assay buffer, and 15 μL of each sample was transferred to a 384-well plate. An equal volume of reaction mix was added to standards and samples, and the plate was incubated at room temperature for 30 minutes, protected from light. Fluorescence was measured at 535 nm excitation and 587 nm emission using a Synergy H1 microplate reader. ATP concentrations (nmol) were calculated from the standard curve and normalized to cell number per well.

### RT-PCR Analyses

Total RNA was isolated from hTCEpi cells by the phenol chloroform method using TRIzol reagent (as described herein an ¹⁰). The cDNA was synthesized using a high-capacity reverse transcription kit (4368814; Applied Biosystems). Gene specific primers were designed using NCBI Primer BLAST and purchased from Integrated DNA Technologies (IDT, San Jose, CA, USA; see the Table 1). Semi-quantitative real time PCR (RT-PCR) was performed using 2×Universal SYBR Green Fast qPCR Mix (RK21203 ABclonal) on a CFX Connect Real Time PCR system (Bio-Rad). Relative gene expression was calculated using the 2\hat{ }-ΔΔCq method with GAPDH as the reference gene and expressed as fold change relative to untreated cells.

TABLE 1
List of Primers Used for RT-PCR Analysis

| Gene | Forward Primer (5′ → 3′) | Reverse Primer (5′ → 3′) |
|---|---|---|
| CPT1 | CTTGCCCTGAGACGGGGATT (SEQ ID | TCCAGCCCAGCACATGAAC (SEQ ID |
| | NO: 1) | NO: 2) |
| CPT2 | GCCACCATGAAAACCCTCAC (SEQ ID | GCCCTGGCCAGCACT (SEQ ID NO: 4) |
| | NO: 3) | |
| CrAT | GACCGTGGTGAAGCCTCTG (SEQ ID | ACCACTCAGACAGCCAGTTC (SEQ ID |
| | NO: 5) | NO: 6) |
| GAPDH | AATGAAGGGGTCATTGATGG (SEQ ID | AAGGTGAAGGTCGGAGTCAA (SEQ ID |
| | NO: 7) | NO: 8) |

### Western Blotting Analyses

The hTCEpi cell lysates were prepared in RIPA buffer supplemented with protease and phosphatase inhibitors (as described herein and ¹⁰). Then, 25 μg to 30 μg of protein were separated on 4% to 15% SDS-PAGE gels, transferred to nitrocellulose membranes, blocked with 5% milk in TBST for 2 hours, and incubated overnight with primary antibodies at 4 °C. After washing, membranes were incubated with RP-conjugated secondary antibodies for 1 hour. Bands were visualized using ECL reagents (ThermoFisher Scientific) on a ChemiDoc imaging system. Band intensities were quantified using ImageJ software, normalized to β-actin, and expressed as fold change relative to untreated controls.

The following antibodies were used: PPARα (sc-398394; Santa Cruz Biotechnology), iNOS (ab3523; Abcam), CPT1 (ab128568; Abcam), CPT2 (A12426; ABclonal), CrAT (PA5-82800; Invitrogen), and β-actin (3700S; Cell Signaling Technology). RP-conjugated goat anti-mouse and goat anti-rabbit secondary antibodies (31430 and 31460; ThermoFisher Scientific).

### Measurement of CPT and CrAT Enzyme Activities

CPT1 and 2 (Total CPT) and CrAT activities were measured in hTCEpi cells using assays adapted from previously published studies²⁴⁻²⁶ Briefly, for the total CPT activity assay, 20 μg of protein was added to each well of a 96-well plate in 200 μL of reaction buffer containing 20 mM HEPES, 1 mM EGTA, 220 mM sucrose, 40 mM KCl, 0.1 mM 5,5′-dithiobis-(2-nitrobenzoic acid) (DTNB), 1.3 mg/mL bovine serum albumin (BSA), and 40 μM palmitoyl-CoA (pH 7.4). For the CrAT activity, 30 μg protein was assayed under identical buffer conditions, except that 40 μM acetyl-CoA was used in place of palmitoyl-CoA. In both assays, reactions were initiated by the addition of 1 mM L-carnitine and incubated at 37 °C for 5 minutes.

Absorbance of 5-thio-2-nitrobenzoic acid (TNB), generated from DTNB, was measured at 412 nm using a Synergy H1 spectrophotometer with pathlength correction applied. Enzymatic activity was calculated using the Beer-Lambert law (A=εcl), where A represents absorbance, ε is the molar extinction coefficient, c is molar concentration, and f is the optical path length through the sample. Calculations were performed using the extinction coefficient for TNB (ε=13,600 M⁻¹cm⁻¹). Total CPT and CrAT activities were expressed as nmol of TNB produced per minute per mg of protein.

### Statistical Analysis

All statistical analyses were performed using GraphPad Prism version 10.0 (GraphPad Software, Inc., San Diego, CA, USA). Data are presented as mean±standard error of the mean (SEM). Group comparisons were performed using ordinary 1-way ANOVA, followed by Holm-Šidák multiple comparisons test. A P value <0.05 was considered statistically significant.

### Results

### L-carnitine Mitigates Hyperosmolarity-Induced Increases in Free Fatty Acid Uptake and Triglyceride Levels

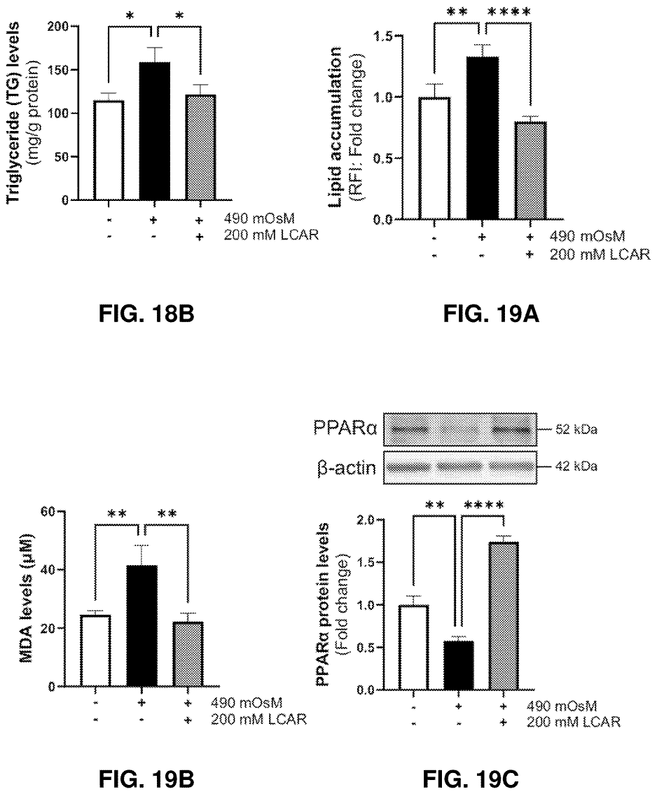

Elevated concentrations of FFAs can disrupt the tear film lipid layer, promoting excessive tear evaporation and contributing to DED pathology.²⁷ To investigate whether L-carnitine modulates hyperosmolarity-induced lipid dysregulation, FFA uptake was measured in hTCEpi cells following exposure to hyperosmolar stress. Treatment with 90 mM NaCl resulted in a 1.08-fold increase in FFA uptake compared with untreated cells. In contrast, cells treated with L-carnitine exhibited significantly reduced FFA uptake under hyperosmolar conditions compared with NaCl alone (FIG. 18A). Consistent with these findings, intracellular TG levels were also increased under hyperosmolar conditions. Exposure to 90 mM NaCl led to a 1.38-fold increase in TG content relative to untreated controls, whereas L-carnitine significantly decreased TG accumulation (FIG. 18B).

### Hyperosmolar Stress-Induced Lipotoxicity is Attenuated by L-Carnitine Supplementation

To evaluate whether L-carnitine mitigates lipid overload, the intracellular lipid accumulation and peroxidation were assessed using Nile Red staining and a malondialdehyde (MDA) assay, respectively. Hyperosmolar conditions significantly increased lipid droplet accumulation relative to untreated cells (1.33-fold), which was significantly attenuated by L-carnitine treatment (0.60-fold relative to NaCl alone; FIG. 19A). Similarly, exposure to hyperosmolar stress resulted in a 1.69-fold increase in MDA levels compared with untreated cells, indicating enhanced lipid peroxidation. In contrast, supplementation with 200 mM L-carnitine significantly reduced MDA levels (0.53-fold) compared to NaCl alone (FIG. 19B).

Given that peroxisome proliferator-activated receptor alpha (PPARα) is an important regulator of mitochondrial fatty acid β-oxidation and lipid homeostasis,^{28,29} it was examined whether its expression was altered by hyperosmolar stress and L-carnitine treatment. Hyperosmolar conditions significantly reduced PPARα protein levels (0.58-fold) relative to untreated controls. Interestingly, L-carnitine treatment increased PPARα levels above baseline (1.78-fold compared with untreated cells; FIG. 19C). Collectively, these findings indicate that hyperosmolarity-induced disruption of lipid homeostasis is attenuated by L-carnitine supplementation.

### Effects of L-Carnitine Treatment on Mitochondrial Oxidative Stress and ATP Levels

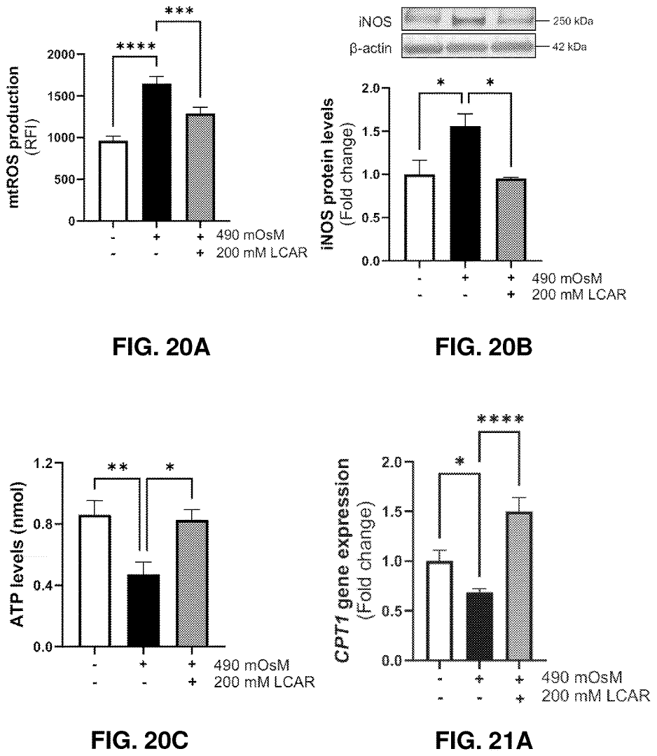

Mitochondria are important sources of intracellular superoxide and important contributors to oxidative stress-mediated cellular injury.³⁰ The mtROS were measured in hTCEpi cells using MitoSOX Red staining. Exposure to 90 mM NaCl resulted in a 1.71-fold increase in mtROS compared with untreated cells, whereas supplementation with L-carnitine significantly reduced mtROS levels (0.78-fold) relative to NaCl alone (FIG. 20A). Consistent with increased oxidative stress, hyperosmolar conditions also significantly elevated inducible nitric oxide synthase (iNOS) protein levels, with the active homodimer (approximately 250 kDa) increased (1.56-fold) relative to untreated cells. L-carnitine significantly reduced iNOS levels (0.61-fold) relative to NaCl alone (FIG. 20B).

Further, total intracellular ATP levels were quantified using a fluorometric ATP assay. Exposure of hTCEpi cells to hyperosmolar conditions significantly reduced ATP levels (0.55-fold) compared with untreated cells. In contrast, L-carnitine supplementation restored ATP levels close to baseline (FIG. 20C). Although the specific metabolic sources contributing to ATP production were not examined, the reduction in mtROS and iNOS levels, together with ATP recovery, suggests that L-carnitine may be associated with preservation of mitochondrial metabolic function in corneal epithelial cells under hyperosmolar stress.

### L-Carnitine Partially Preserves Activity of Carnitine Shuttle Enzymes Under Hyperosmolar Stress

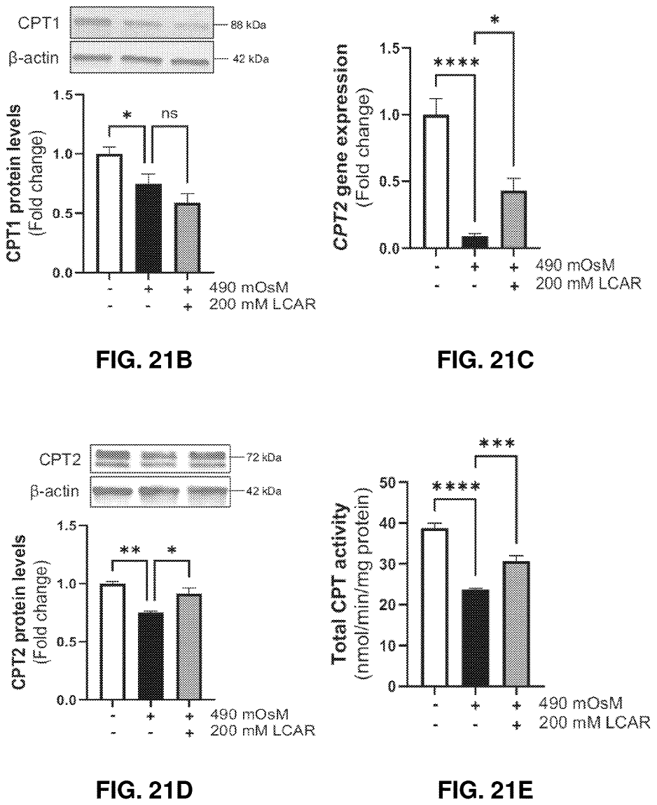

To determine whether hyperosmolar stress disrupts the mitochondrial carnitine shuttle pathway, three enzymes (CPT1, CPT2, and CrAT) involved in fatty acid transport were examined. Exposure to 90 mM NaCl significantly reduced CPT1 mRNA expression (0.69-fold) and protein levels (0.75-fold) relative to untreated cells (FIGS. 21A, 21B). Similarly, CPT2 mRNA expression (0.09-fold) and protein levels (0.75-fold) were also significantly decreased under hyperosmolar conditions compared with untreated cells (FIGS. 21C, 21D). In addition, total CPT enzymatic activity was significantly reduced (0.61-fold) in the cells under hyperosmolar stress as compared with untreated cells (23.68 vs. 38.74 nmol/min/mg protein; FIG. 21E). Pre-treatment with 200 mM L-carnitine partially mitigated these effects, by restoring CPT1 and CPT2 mRNA expression, CPT2 protein levels, and total CPT activity (30.67 nmol/min/mg protein).

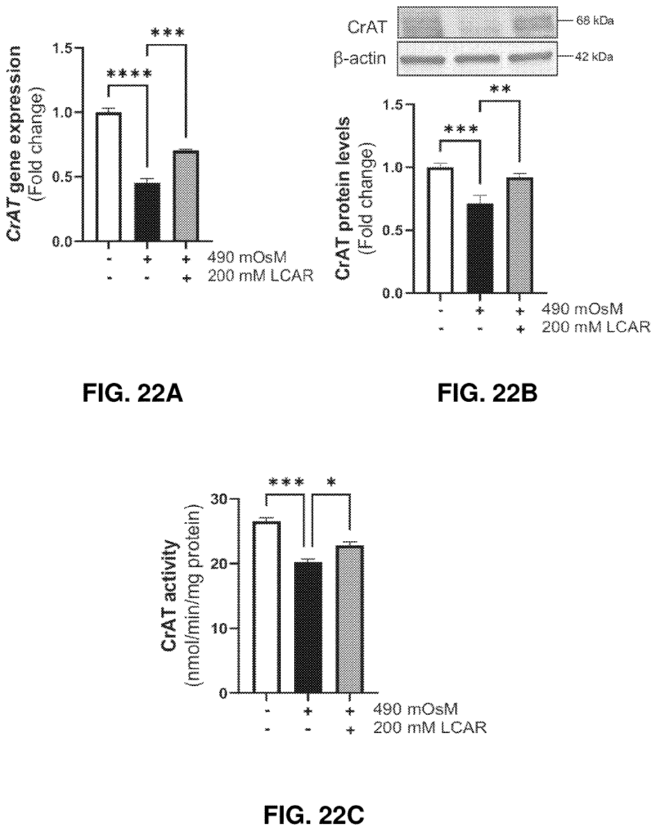

Hyperosmolar stress also significantly reduced CrAT mRNA (0.45-fold) and protein levels (0.71-fold) relative to untreated cells (FIGS. 22A, 22B). Similar to the effects observed for CPT enzymes, pre-treatment with L-carnitine partially prevented these reductions, with CrAT protein levels restored nearly to baseline. CrAT enzymatic activity was also reduced under hyperosmolar stress compared with untreated cells (20.27 vs. 26.57 nmol/min/mg protein), whereas L-carnitine partially restored CrAT activity (22.81 nmol/min/mg protein; FIG. 22C). Collectively, these findings indicate that hyperosmolar stress impairs the mitochondrial carnitine shuttle pathway, whereas L-carnitine preserves the activity of these enzymes under osmotic stress conditions.

### DISCUSSION

Dry eye disease (DED) is a prevalent, multifactorial disorder of the ocular surface characterized by tear film instability, hyperosmolarity, inflammation, and epithelial damage that affects visual function and quality of life for millions of people worldwide.^{1,2} Clinically, DED is categorized into aqueous-deficient dry eye (ADDE) and evaporative dry eye (EDE) subtypes, distinguished by their pathophysiological mechanisms.¹ ADDE arises from impaired lacrimal gland secretion, leading to reduced aqueous tear volume, whereas EDE results from excessive tear evaporation due to meibomian gland dysfunction or environmental stressors.^{1,3} These subtypes frequently coexist, and thus diagnosis and treatment of DED become challenging.^{3,4}

Tear hyperosmolarity is recognized as both a diagnostic biomarker and a principal pathological factor in DED.^{3,5} Under healthy physiologic conditions, tear osmolarity is tightly maintained near 302±8 to 10 mOsm/L, whereas patients with DED typically present with values exceeding 308 mOsm/L, reaching up to 336±22 mOsm/L in severe disease.^{6,7} Elevated osmolarity acts as a potent stressor to the corneal epithelium, triggering inflammation, oxidative stress, and apoptosis.^{3,7-9} The corneal epithelium functions as both a protective barrier and the eye's primary refractive interface; therefore, hyperosmolarity-induced damage at this site directly contributes to visual disturbance and disease progression (as described herein and^{7,10}). Further, increasing evidence indicates that tear hyperosmolarity not only triggers inflammatory and apoptotic signaling but also disrupts mitochondrial homeostasis, an important determinant of epithelial health.^{7,11}

Hyperosmolar conditions are known to elevate intracellular ionic strength, induce oxidative stress, and activate stress-response pathways, such as MAPK and NF-κB, which collectively impair mitochondrial function.¹² These changes promote an imbalance between oxidant production and antioxidant defense, resulting in excess mitochondrial reactive oxygen species (mtROS) generation and loss of mitochondrial membrane potential.¹³ In corneal epithelial cells, sustained osmotic stress reduces mitochondrial ATP production and alters bioenergetic profiles, suggesting a shift toward metabolic inefficiency.^{14,15} The resulting mitochondrial dysfunction exacerbates inflammation, barrier instability, and cell death, well-known hallmarks of DED pathology.^{3,16} Thus, identifying the molecular relationship between hyperosmolarity-induced stress signaling and mitochondrial injury is important for understanding the progression of epithelial damage associated with DED.

Mitochondrial β-oxidation, the principal pathway for fatty acid catabolism, depends on the carnitine shuttle system, which transports long-chain fatty acids (LCFAs) into the mitochondria for energy production.¹⁷ This system is regulated by carnitine palmitoyltransferase (CPT1 and CPT2) and carnitine acetyltransferase (CrAT) enzymes, which collectively control the balance between acyl-CoA and acetyl-CoA pools within the mitochondrial matrix. Impairment of this system reduces β-oxidation efficiency and ATP synthesis, compromising cellular energy homeostasis.¹⁷ Clinically, reduced levels of carnitine and its acyl derivatives have been reported in the tears of patients with DED.¹⁸

L-carnitine, a naturally occurring osmoprotectant, stabilizes corneal epithelial cells exposed to hyperosmolar environments without disrupting normal metabolism.^{19,20} Studies have also shown that L-carnitine mitigates hyper-osmolarity-induced inflammation, preserves cell viability, and maintains barrier integrity in human corneal epithelial cells.^{20,21} In addition, it was demonstrated that L-carnitine partially restores epithelial adherens junctions, improves barrier function, and enhances wound healing capacity under hyperosmolar stress (as described herein and¹⁰). Although hyperosmolarity can induce mitochondrial damage, the relationship among lipid instability, mitochondrial dysfunction, and carnitine homeostasis remains poorly understood. Therefore, this study investigated the molecular mechanisms by which L-carnitine restores mitochondrial function and lipid homeostasis in human corneal epithelial cells exposed to hyperosmolar stress.

Loss of tear film homeostasis and increased hyperosmolarity are important drivers of inflammation and ocular surface damage in dry eye disease.^{1,31,32} Several in vitro and in vivo studies have demonstrated that hyperosmolar conditions activate pro-inflammatory signaling and impair corneal epithelial wound healing and cell survival (as described herein and^{10,12,33-35}). Hyperosmolarity can disrupt corneal epithelial barrier function, whereas L-carnitine, an osmoprotectant, preserves barrier integrity and promotes epithelial wound healing (as described herein and¹⁰)

Clinical studies have also reported reduced carnitine levels in the tears of patients with DED.¹⁸ In addition, L-carnitine, betaine, and taurine have been shown to suppress hyperosmolarity-induced inflammatory mediators, such as TNF-α and IL-1β, and attenuate oxidative damage in human corneal epithelial cells.^{20,36} In the present study, a 6-hour hyperosmolarity model was used to examine the acute effects of increased osmolarity, an approach used in several in vitro DED studies.^{37,38} This short term exposure to NaCl has been shown to mimic clinically relevant features of DED, including pro-inflammatory cytokine production, mitochondrial dysfunction, oxidative stress, and cell death in corneal epithelial cells.^{16,32} The present study showed that exposure to 90 mM NaCl disrupted mitochondrial carnitine metabolism, as indicated by decreased levels of CPT1, CPT2, and CrAT enzymes. Hyperosmolarity induced free fatty acid uptake, neutral lipid and TG accumulation, lipid peroxidation, mtROS generation, and ATP depletion. Supplementation with L-carnitine restored the carnitine-pathway enzymes, reduced lipid dysregulation, lowered mitochondrial oxidative stress, and partially recovered ATP levels.

Hyperosmolar stress can disrupt lipid homeostasis in the corneal epithelium, contributing to cellular damage and cell death. Exposure to hyperosmolar conditions (approximately 500 mOsM) induces lipid droplet accumulation, ceramide and TG buildup, lipidomic remodeling, and ferroptotic cell death in corneal epithelial cells.^{11,39,40} Lipidomic studies further demonstrate a dose-dependent increase in TG levels following NaCl-induced hyperosmolarity (50 mM-40 mM), and increased number and size of lipid droplets.^{11,40} These lipidomic alterations disrupt membrane integrity and metabolic homeostasis, leading to lipotoxicity, in which excessive neutral lipids undergo peroxidation, damaging mitochondrial membranes and impairing oxidative energy metabolism.⁴¹ This process may suggest a feed-forward loop where lipid peroxidation exacerbates mitochondrial oxidative stress, further disrupting β-oxidation and ATP synthesis.

Significantly elevated TG content and lipid droplet accumulation was observed in NaCl-treated hTCEpi cells. TGs stored within lipid droplets are highly susceptible to oxidative degradation. Excessive TG accumulation predisposes cells to lipid peroxidation, generating cytotoxic aldehydes such as MDA and 4-hydroxynonenal (4-HNE), which promote inflammation and mitochondrial damage. Lipid peroxidation has been reported in corneal epithelial cells under hyperosmolar stress.^{9,42,43} For example, Deng et al. (2015) reported that exposure of human corneal epithelial cells to 400 to 450 mOsM media significantly increased MDA and 4-HNE levels, enhanced ROS production, and reduced antioxidant enzyme activity.⁹ Similarly, exposure to 4-HNE exacerbated ROS accumulation, downregulated superoxide dismutase 1 (SOD1), and activated NF-κB signaling, thereby upregulating IL-6 and IL-8 expression.⁴² In this study, MDA levels were significantly elevated following NaCl treatment, supporting the contribution of oxidative lipid injury to the pathophysiology of DED. Notably, L-carnitine treatment attenuated TG accumulation, lipid droplet formation, and MDA levels, indicating that L-carnitine and related osmoprotectants mitigate oxidative damage in corneal epithelial cells.

Carnitines play an important role in transporting LCFAs into mitochondria for β-oxidation, a key pathway for ATP generation. CPT1, located on the outer mitochondrial membrane, and CPT2, on the inner membrane, catalyze the conversion of acyl-carnitines, thereby facilitating the translocation of LCFAs into the mitochondrial matrix.^{44,45} CrAT regulates mitochondrial acetyl-CoA homeostasis by reversibly converting acetyl-CoA to acetyl carnitine, thereby maintaining the acetyl-CoA/CoA balance. In the present model, hyperosmolar stress suppressed total CPT and CrAT enzyme activities, which likely resulted in reduced LCFA import, cytosolic accumulation of acyl-intermediates, impaired β-oxidation, and increased mitochondrial metabolic burden.^{46,47} In the present study, it was found that treatment with 90 mM NaCl significantly reduced both CPT1 mRNA and protein levels, whereas L-carnitine supplementation was able to preserve only mRNA levels. This discrepancy between mRNA and protein levels may be due to translational control, altered protein turnover, or post-translational regulation. In addition, the total CPT activity that was restored after LCAR treatment reflects the combined function of both CPT1 and CPT2 enzymes.

The downregulation of CPT1/2 and CrAT enzymes may reflect transcriptional repression mediated by stress-activated signaling, including MAPK and NF-κB pathways, which are upregulated under hyperosmolar conditions and regulate genes involved in lipid metabolism.¹² Loss of CrAT activity can further promote acyl-CoA accumulation, inhibit key oxidative enzymes and exacerbate mitochondrial dysfunction. Collectively, impaired carnitine-mediated fatty acid trafficking may promote lipid accumulation, TG deposition, and oxidative damage to the electron transport chain, ultimately resulting in reduced ATP synthesis.⁴⁸

The suppression of carnitine pathway enzymes under hyperosmolar stress may also involve dysregulation of PPARα, a transcriptional activator of CPT1 and CrAT that supports mitochondrial β-oxidation and epithelial repair in ocular tissues.^{49,50} The present findings suggest that L-carnitine supplementation prevents the decrease in PPARα expression under osmotic stress. In parallel, elevated iNOS expression is associated with delayed corneal repair and heightened inflammation.

### Conclusions

This study demonstrates that hyperosmolar stress disrupts mitochondrial carnitine trafficking and induces mtROS generation and lipotoxicity in corneal epithelial cells. L-carnitine supplementation restores the activity of carnitine shuttle enzymes, improves mitochondrial function, and mitigates lipid accumulation and oxidative injury. Collectively, these findings highlight a role of the mitochondrial carnitine pathway as a promising therapeutic strategy to protect the ocular surface from hyperosmolar injury in DED.

### REFERENCES

1. Craig, et al. (2017), Ocul Surf(3): 276-283.
2. Stapleton, et al. (2017), Ocul Surf(3): 334-365.
3. Bron, et al. (2017), Ocul Surf(3): 438-510.
4. Golden (2025), StatPearls: Treasure Island.
5. Craig, et al. (2017), Ocul Surf(4): 802-812.
6. Lemp, et al. (2011), Am J Ophthalmol(5): 792-798.
7. Baudouin, et al. (2013), Ocul Surf(4): 246-258.
8. Igarashi, et al. (2014), Cornea(12): 1342-1347.
9. Deng, et al. (2015), PLoS One(5):e0126561.
10. Kontoh-Twumasi, et al. (2025), Exp Eye Res: 110499.
11. Magny, et al. (2020), Biochim Biophys Acta Mol Cell Biol Lipids(9): 158728.
12. Luo, et al. (2005), Eye Contact Lens(5): 186-193.
13. Png, et al. (2011), J Cell Physiol(3): 693-699.
14. Li, et al. (2017), PLoS One(3):e0174437.
15. Hou, et al. (2024), Front Pharmacol: 1407659.
16. Stuard Sambhariya, et al. (2023), J Biol Chem(11): 105239.
17. Sharma, et al. (2009), Drug Discov Today Dis Mech(1-4):e31-e39.
18. Pescosolido, et al. (2009), Optom Vis Sci(2):E132-E138.
19. Chen, et al. (2013), Invest Ophthalmol Vis Sci(9): 6287-6297.
20. Hua, et al. (2015), Curr Eye Res(7): 657-667.
21. Hua, et al. (2015), Invest Ophthalmol Vis Sci(9): 5503-5511.
22. Robertson, et al. (2005), Invest Ophthalmol Vis Sci(2): 470-478.
23. Folch, et al. (1957), J Biol Chem(1): 497-509.
24. Bieber, et al. (1972), Anal Biochem(2): 509-518.
25. Ling, et al. (2012), Nutr Metab (Lond)(1): 66.
26. Mao, et al. (2023), Nat Metab(8): 1382-1394.
27. Arciniega, et al. (2011), Exp Eye Res(4): 452-459.
28. Makowski, et al. (2009), FASEB J(2): 586-604.
29. Patsouris, et al. (2006), Endocrinology(3): 1508-1516.
30. Brand, et al. (2004), Free Radic Biol Med(6): 755-767.
31. Zuo, et al. (2024), Invest Ophthalmol Vis Sci(14): 12.
32. Bogdan, et al. (2021), Invest Ophthalmol Vis Sci(7): 11.
33. Li, et al. (2024), Ocul Surf:182-191.
34. Shen, et al. (2023), Exp Eye Res: 109418.
35. Li, et al. (2020), Exp Eye Res: 108113.
36. Lopez-Cano, et al. (2021), Exp Eye Res: 108723.
37. Stuard, et al. (2022), Int J Mol Sci(7): 4066.
38. Guindolet, et al. (2022), Int J Mol Sci(9): 4528.
39. Zuo, et al. (2022), Invest Ophthalmol Vis Sci(10): 3.
40. Robciuc, et al. (2012), J Lipid Res(11): 2286-2295.
41. Schrauwen, et al. (2010), Biochim Biophys Acta(3): 266-271.
42. Liu, et al. (2020), Graefes Arch Clin Exp Ophthalmol(8): 1717-1725.
43. Li, et al. (2016), Sci Rep: 19408.
44. Choi, et al. (2024), Cell Rep(12): 115006.
45. Kienesberger, et al. (2014), BMC Genomics(1): 514.
46. Seiler, et al. (2015), Cell Metab(1): 65-76.
47. Schlaepfer, et al. (2020), Endocrinology(2):bqz046.
48. Idrovo, et al. (2012), J Surg Res(1): 157-164.
49. Song, et al. (2010), Mol Cell Endocrinol(1-2): 54-63.
50. Goto, et al. (2011), J Lipid Res(5): 873-884.
51. Wu, et al. (2024), Mol Vis: 410-420.
52. Ding, et al. (2014), Am J Pathol(10): 2709-2720.
53. Liang, et al. (2023), Proc Natl Acad Sci USA(13):e2217576120.
54. Sun, et al. (2024), Exp Eye Res: 109886.

### Example 4: L-Carnitine Partially Restores Adherens Junction Integrity and Promotes Wound Healing in Human Corneal Epithelial Cells Exposed to Hyperosmolar Stress

### Materials and Methods

### Cell Culture and Hyperosmolar Model

Telomerase-immortalized human corneal epithelial cells (hTCEpi) developed and characterized by Dr. Robertson and colleagues (Robertson et al., 2005) were cultured in complete Keratinocyte Growth Medium-2 (KGM, C-20111, PromoCell, Heidelberg, Germany) supplemented with 0.15 mM calcium chloride (C-34006, PromoCell), bovine pituitary extract (0.0004 mL/mL), recombinant human epidermal growth factor (0.125 ng/mL), recombinant human insulin (5 μg/mL), hydrocortisone (0.33 μg/mL), epinephrine (0.39 μg/mL), and human holo-transferrin (10 μg/mL). Cells were seeded at 3×10⁵ cells per well in 12-well plates (Corning Incorporated, Kennebunk, ME, USA) and allowed to adhere overnight. Prior to experiments, cells were transferred to KGM media without supplements (basal) for 24 hours. To induce hyperosmolar stress, the culture medium was supplemented with 70, 90, or 120 mM NaCl, resulting in final osmolarities of approximately 450, 490, and 550 mOsm/L, respectively, as previously described (Luo, Li, & Pflugfelder, 2007). The hyperosmolar hTCEpi cells were treated with/without 200 mM L-carnitine supplementation (8400920025, Millipore Sigma, Burlington, MA, USA) to investigate its therapeutic potential.

### Epithelial Barrier Function Measurement

Trans-epithelial electrical resistance (TER) was measured using an ECIS Z-Theta instrument (Applied Biophysics, Inc., Troy, NY, USA) to evaluate epithelial barrier function in hTCEpi cells exposed to increasing hyperosmolar stress with and without L-carnitine supplementation. The 96-well ECIS arrays (96W1E+) were pre-coated with L-cysteine (10 μM; Alfa Aesar, Haverhill, MA, USA) and fibronectin (10 μg/mL; Thermo Fisher, Waltham, MA, USA), and baseline readings were stabilized in cell-free media. hTCEpi cells were seeded at 70,000 cells per well in complete KGM media until confluency and then switched to basal media for 24 hours. Hyperosmolar stress was induced by the addition of 70, 90, or 120 mM NaCl. Epithelial barrier function was assessed by recording resistance readings at 4000 Hz, every 5-minute for 24 hours and data normalized to the initial resistance values.

### Wound Healing Assay

Wound healing and cell migration were assessed using the automated wounding function of the ECIS Z-Theta system with 96W1E+ electrode arrays. Unlike the conventional scratch assay, which involves manually disrupting the monolayer with pipette tips or needles, ECIS offers advantages, including eliminating user variability, preserving the extracellular matrix, restricting the wound area to a defined region, and providing real-time, quantitative monitoring of both cell migration and barrier function. The coating, stabilization, cell seeding, and treatment protocols described above in the barrier function assay were used. Uniform wounds were generated by applying a 5000 μA current for 30 seconds across two circular electrodes (radius: 177 μm; area: 0.256 mm²). This method has been shown to selectively kill cells on the electrodes (Ebrahim et al., 2023; Ebrahim et al., 2022; Gamal et al., 2015; Keese et al., 2004; Vereb et al., 2016) without physically dislodging them as in a scratch assay. The automated electric fence function of ECIS was also applied for 1 minute to prevent immediate hTCEpi cell migration to the wounded areas. Wound closure was monitored every 5 minutes for 24 hours, with resistance values normalized to the baseline readings. Migration rate (V) was determined using the formula V=R/T, where R is the known radius of the electrodes and T is the migration time calculated from the TER drop at wounding to its recovery.

### RT-PCR Analysis

RNA was extracted from hTCEpi cells by the phenol-chloroform method using TRIzol reagent (Invitrogen, Carlsbad, CA, USA), followed by cDNA synthesis with the High-Capacity cDNA Reverse Transcriptase Kit (Applied Biosystems, Foster City, CA, USA). Primers were designed using NCBI Primer-BLAST and purchased from Integrated Device Technology (San Jose, CA, USA) (Table 2). RT-PCR was performed using 2×Universal SYBR Green Fast qPCR Mix (RK21203, Abclonal, USA) and Bio-Rad CFX Connect Thermocycler. Relative gene expression was calculated as 2^{-ΔΔCq} relative to GAPDH expression and expressed as fold change relative to untreated.

TABLE 2
List of Primers Used in the Study

| Gene | Forward Primer (5′ → 3′) | Reverse Primer (5′ → 3′) |
|---|---|---|
| E-cadherin (CDH1) | GCTGGACCGAGAGAGTTTCC (SEQ | GGTGTATACAGCCTCCCACG |
| | ID NO: 9) | (SEQ ID NO: 10) |
| β-catenin (CTNNB1) | ATTGTCCACGCTGGATTTTC (SEQ | TCGAGGACGGTCGGACT (SEQ ID |
| | ID NO: 11) | NO: 12) |
| α-catenin (CTNNA1) | GCTCACAGTGTAATAGGGGAGA | TGGACAGCAGTCATTTTGCAG |
| | (SEQ ID NO: 13) | (SEQ ID NO: 14) |
| p120-catenin | AGAGCGTTACCAAGAGGCAG | GGAGAACCACTCATCTTTGCC |
| (CTNND1) | (SEQ ID NO: 15) | (SEQ ID NO: 16) |
| Glyceraldehyde 3- | AATGAAGGGGTCATTGATGG | AAGGTGAAGGTCGGAGTCAA |
| phosphate | (SEQ ID NO: 17) | (SEQ ID NO: 18) |
| dehydrogenase | | |
| (GAPDH) | | |

### Western Blotting

Cell lysates were in RIPA buffer (AKR191, Cell Biolabs, Inc., San Diego, CA, USA) containing protease inhibitor (P8340, Sigma-Aldrich, St. Louis, MO, USA) and phosphatase inhibitor (P5726, Sigma-Aldrich) and lysed by sonication followed by centrifugation. Protein concentrations were determined by Bradford Protein Assay (23200, Thermo Scientific, Waltham, MA, USA). Equal amounts of protein (20 μg-30 μg) were separated on 4-20% SDS-PAGE gels and transferred to nitrocellulose membranes. Membranes were blocked with 5% non-fat milk dissolved in tris-buffered saline with 0.1% Tween-20 (TBS-T) and incubated overnight with primary antibodies against E-cadherin and β-catenin (1:1000, #3195 and #8480 respectively, Cell Signaling Technology, Danvers MA, USA). Membranes were washed and then developed using ECL substrate (Thermo Scientific), and bands were visualized using the ChemiDoc Imaging System (Bio-Rad). Densitometric analysis was performed using ImageJ (NIH), and protein levels were normalized to β-actin (1:5000, 8H10D10, Cell Signaling Technology). Data were presented as fold change relative to untreated hTCEpi cells.

### Immunofluorescence

hTCEpi cells were cultured on 96-well plates (Greiner 655090, Fisher Scientific, Waltham, MA) at a density of 70,000 cells per well and treated with 90 mM NaCl (490 mOsM) with or without L-carnitine as described above. Cells were washed with DPBS, fixed in 4% paraformaldehyde, permeabilized with 0.3% Triton X-100, and blocked with 3% BSA at room temperature. Primary antibodies against E-cadherin and β-catenin were applied at 1:1,000 and 1:400 dilutions, respectively, and Alexa Fluor 488 and 546-conjugated secondary antibodies (1:500, Invitrogen, Carlsbad, CA) were used for fluorescence detection. Images were acquired using a Leica STELLARIS Confocal Microscope (Buffalo Grove, IL) at 40× magnification and quantified using ImageJ software (NIH) measuring the relative fluorescence intensity (RFI) normalized to total cell count.

### Statistical Analysis

Statistical analyses were performed using GraphPad Prism software version 10 (GraphPad Software, Inc., San Diego, CA, USA), using one-way ANOVA with Tukey's post hoc test or a student's t-test. P-value of <0.05 was considered significant and data are expressed as mean±standard deviation (SD).

### Results

### Hyperosmolarity Induces a Dose-Dependent Decline in Epithelial Barrier Function

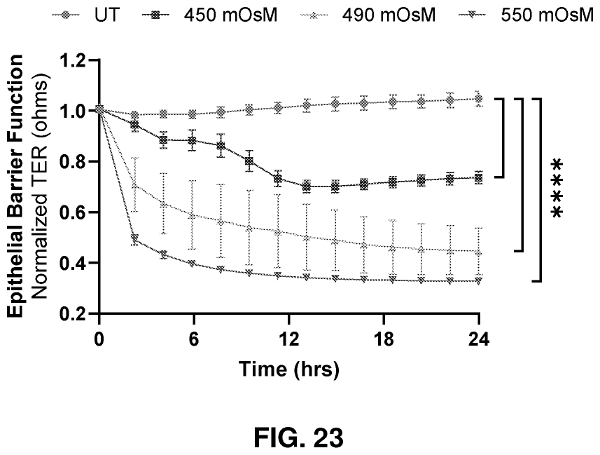

Corneal epithelial cells exposed to increasing concentrations of NaCl (70, 90, and 120 mM, corresponding to final osmolarities of ~450, 490, and 550 mOsm/L, respectively) for 24 hours exhibited a dose-dependent reduction in trans-epithelial resistance (TER), as measured by ECIS apparatus (FIG. 23). A significant decline of 30%, 54%, and 69% was observed in cells treated with 70, 90, and 120 mM NaCl respectively, relative to untreated controls. These findings indicate that elevated hyperosmolarity disrupts corneal epithelial barrier integrity.

### Hyperosmolar Stress Reduces Adherens Junction Protein Levels in Corneal Epithelial Cells

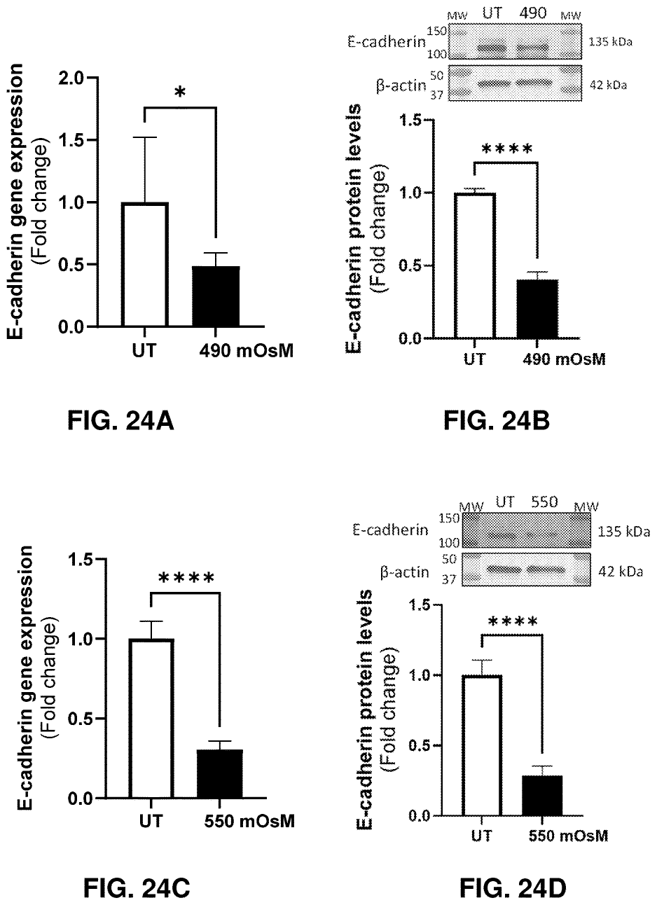

To investigate molecular mechanisms underlying barrier dysfunction, the expression of important adherens junction components, E-cadherin and β-catenin was analyzed. RT-PCR analysis showed significant downregulation in E-cadherin expression with a 0.49-fold decrease in the 490 mOsM group and a 0.31-fold decrease in the 550 mOsM group (FIG. 24A and FIG. 24C). Western blot analysis revealed corresponding decreases in protein levels of 0.41-fold and 0.28-fold in the 490 and 550 mOsM groups, respectively (FIGS. 24B and 24D; data not shown).

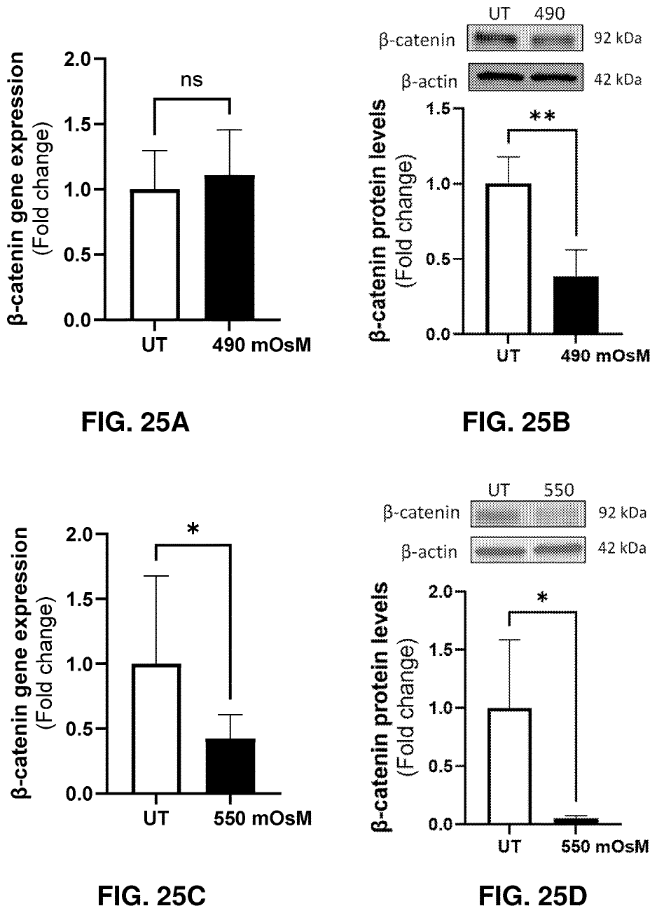

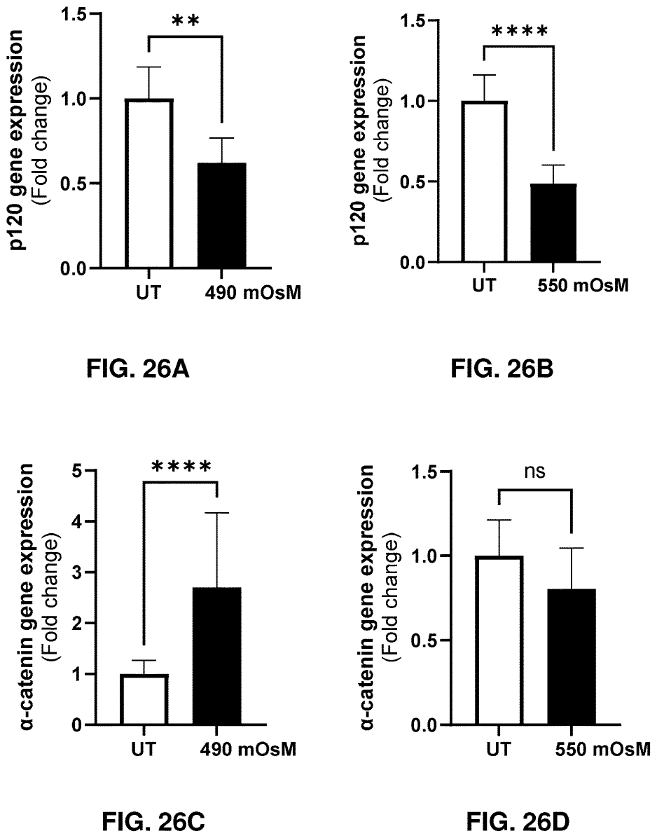

β-catenin RNA levels remained unchanged at 490 mOsM but decreased significantly by 0.42-fold at 550 mOsM (FIG. 25A and FIG. 25C). β-catenin protein levels decreased by 0.38-fold in 490 mOsM and 0.05-fold in the 550 mOsM groups (FIG. 25B and FIG. 25D). Analysis of other adherens junction proteins revealed that p120-catenin RNA expression decreased by 0.62-fold and 0.49-fold in 490 and 550 mOsM, respectively (FIG. 26A and FIG. 26B). In contrast, α-catenin expression increased by 2.7-fold at 490 mOsM but remained unchanged at 550 mOsM (FIG. 26C and FIG. 26D).

### L-Carnitine Supplementation Partially Restores Hyperosmolarity-Induced Loss of Adherens Junction Protein Integrity

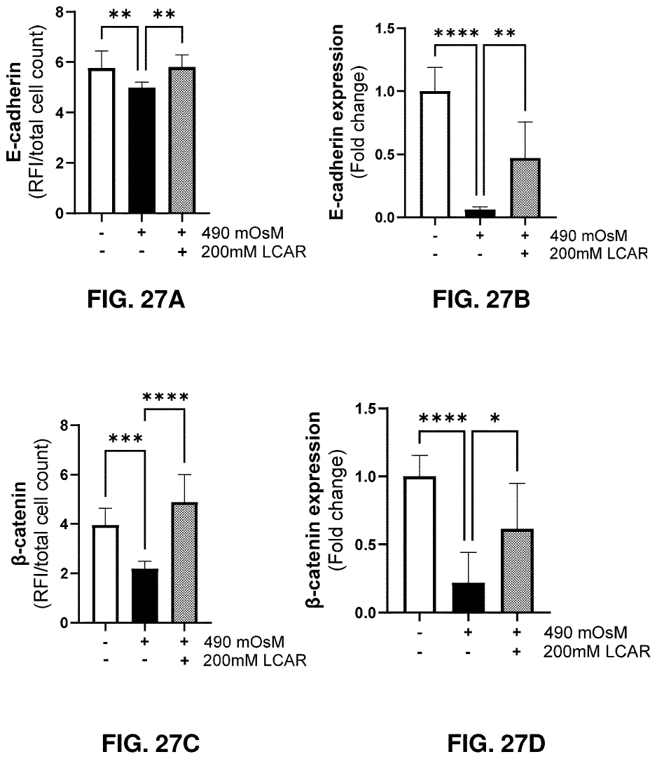

It was examined whether L-carnitine could protect adherens junction integrity under hyperosmolar stress. Immunofluorescence analysis revealed reduced fluorescence intensity of E-cadherin and β-catenin in cells exposed to 490 mOsM. L-carnitine supplementation restored fluorescence levels to near control values (0.99-fold and 1.23-fold, respectively) (FIG. 27A, FIG. 27C). Our RT-PCR analysis showed that L-carnitine enhanced E-cadherin expression by 7.56-fold and β-catenin expression by 2.8-fold as compared to cells treated with 490 mOsM alone (FIG. 27B and FIG. 27D). These findings indicate that L-carnitine mitigates the hyperosmolarity-induced damage and supports adherens junction stability and epithelial barrier function.

### L-Carnitine Partially Improves Epithelial Barrier Function and Wound Healing Under Hyperosmolar Stress

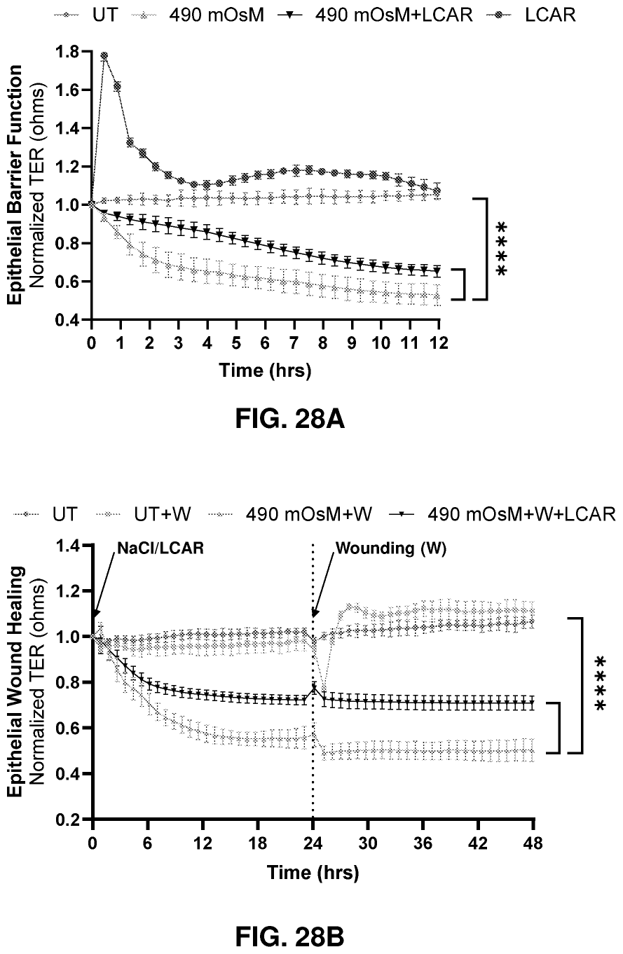

To study the osmoprotective effects of L-carnitine on corneal epithelial barrier integrity under hyperosmolar conditions, TER analysis was performed. Cells exposed to 490 mOsM exhibited a significant reduction in corneal epithelial cell resistance. However, supplementation with 200 mM L-carnitine significantly enhanced TER, partially restoring epithelial barrier function (FIG. 28A).

Furthermore, the ability of the corneal epithelial cells to repair themselves after injury was evaluated using the ECIS wound healing assay, which continuously measures impedance over time, reflecting both cell migration into the wounded area and the reformation of intercellular junctions. After induction of the electric wound, the untreated cells exhibited a substantial drop in TER, which was quickly recovered, corresponding to a migration rate (V) of 59 μm/hr, indicating efficient repair. With 490 mOsM exposure, TER readings dropped considerably, and migration was markedly delayed, with a reduced migration rate of 7.6 μm/hr. Interestingly, the administration of L-carnitine protected hTCEpi cells from the effects of the electrical wound, as TER measurements remain stable despite hyperosmolar conditions (FIG. 28B). Together, these findings suggest that hyperosmolar stress severely impairs the corneal epithelium's intrinsic repair mechanisms while L-carnitine may protect epithelial cells from damage and promote repair.

### DISCUSSION

Adherens junction proteins are important for maintaining cell-to-cell adhesion in corneal epithelial cells (Kimura et al., 2013; Ma et al., 2021). These proteins include E-cadherin and catenins (α-catenin, β-catenin, and p120-catenin), which work together to maintain epithelial function and regulate various signal transduction pathways. Among these, E-cadherin and β-catenin play an important role in epithelial cell proliferation and migration (Maltseva et al., 2013; Wang et al., 2025).

Despite advances in understanding the molecular pathways involved in DED, the precise mechanisms by which hyperosmolarity disrupts adherens junction complex and modulates wound healing in the corneal epithelium remain unclear. This study investigated the effect of hyperosmotic stress on adherens junction proteins and wound healing using an in vitro DED model. It also evaluated therapeutic potential of L-carnitine, a candidate osmoprotectant (Chen et al., 2013; Hua, Deng, et al., 2015; Hua, Su, et al., 2015; Khajavi et al., 2014), in restoring corneal epithelial barrier function and modulating its molecular targets.

Ocular surface damage in dry eye disease is associated with increased tear osmolarity, epithelial barrier dysfunction, and impaired wound healing (Guzman-Aranguez, Perez de Lara, & Pintor, 2017; Pflugfelder et al., 2005; Redfern et al., 2013). In this study, using the telomerase-immortalized human corneal epithelial (hTCEpi) cells, the effects of hyperosmolar stress on adherens junction proteins, wound healing, and corneal epithelial integrity were investigated. Additionally, the therapeutic potential of L-carnitine as an osmoprotectant to mitigate the adverse effects of hyperosmolarity was evaluated.

The findings of the present study indicate that hyperosmolar stress leads to a progressive decline in corneal epithelial barrier function. Chronic, unmanaged DED can cause a gradual breakdown of the corneal epithelial barrier, increasing the risk of microbial infections and environmental insults that further exacerbate ocular surface pathology. Furthermore, the present results demonstrate that E-cadherin, β-catenin, and p120-catenin were significantly reduced under hyperosmolar conditions, highlighting their role in corneal epithelial homeostasis.

Several studies have focused on the role of other junctional proteins in various DED models. For example, elevated matrix metalloproteinase (MMP)-9 has been shown to degrade occludin, a tight junction protein, contributing to increased corneal epithelial permeability in mouse models of DED (Pflugfelder et al., 2005). Similarly, ZO-1 disruption was observed in corneal epithelial cells exposed to 450 mOsM hyperosmolar stress (Li et al., 2024). In the present study, on the focus was on adherens junction proteins due to their role in regulating cell-cell adhesion through the formation of the cadherin/catenin complex, which is linked to actin cytoskeleton and contributes to the epithelial barrier integrity (Giasson et al., 2014; Kimura et al., 2013). E-cadherin/β-catenin complex regulates several signal transduction pathways, including the PI3K/Akt, NF-κB and Wnt/β-catenin signaling, which are involved in important biological processes such as proliferation and wound healing (Liu et al., 2022; Van den Bossche et al., 2012; Wang et al., 2025).

In this study, an electrically induced wound healing assay was employed that provides real time assessment of cell migration. The results demonstrate that hyperosmolar stress impairs migration of corneal epithelial cells, compromising their ability to repair ocular surface damage and exacerbating corneal barrier dysfunction.

Current DED therapies primarily offer symptomatic relief, including artificial tears, anti-inflammatory drugs, and tear retention strategies, with emerging options such as intense pulse light therapy (Shen et al., 2023; Yurttaser Ocak et al., 2020). L-carnitine aids in transporting long-chain fatty acids into the mitochondria for β-oxidation(Caballero-Garcia et al., 2023). It has also been studied as an osmoprotectant where it counters the effects of osmotic imbalance between cells and their environment (Khajavi et al., 2014). Studies have shown that L-carnitine is effective in suppressing inflammation and reducing oxidative stress in corneal epithelial cells exposed to hyperosmotic stress with the exact mechanism not known (Chen et al., 2013; Hua, Deng, et al., 2015; Hua, Su, et al., 2015).

Overall, the present studies demonstrate the therapeutic potential of L-carnitine supplementation to improve corneal epithelial barrier function, restoring adherens junction protein levels, and enhancing wound healing disrupted by NaCl-induced hyperosmolar stress.

### Conclusion

This study demonstrates that hyperosmolarity disrupts adherens junction proteins, leading to corneal epithelial barrier dysfunction and impaired wound healing. The present findings suggest that L-carnitine supplementation can partially repair the disrupted cadherin-catenin complex and restore barrier function. Thus, the present studies demonstrate the long-term protective effects of L-carnitine in animal models and combinatorial therapies targeting inflammatory and oxidative pathways can further advance treatment strategies for DED.

### REFERENCES

Bian, et al. (2015), Invest Ophthalmol Vis Sci(8): 4908-4918.
Bildin, et al. (2003), J Membr Biol(1): 1-13.
Caballero-Garcia, et al. (2023), Nutrients(11).
Chen, et al. (2013), Invest Ophthalmol Vis Sci(9): 6287-6297.
Craig, et al. (2017), Ocul Surf(4): 802-812.
Ebrahim, et al. (2023), Biosensors (Basel)(11).
Ebrahim, et al. (2022), Sci Rep(1): 14126.
Farrand, et al. (2017), Am J Ophthalmol: 90-98.
Gamal, et al. (2015), Biosens Bioelectron: 445-455.
Giasson, et al. (2014), Mol Vis: 386-394.
Guindolet, et al. (2022), Int J Mol Sci(9).
Guzman-Aranguez, et al. (2017), Purinergic Signal(2): 249-258.
Hua, et al. (2015), Invest Ophthalmol Vis Sci(9): 5503-5511.
Hua, et al. (2015), Curr Eye Res(7): 657-667.
Keese, et al. (2004), Proc Natl Acad Sci USA(6): 1554-1559.
Khajavi, et al. (2014), Cell Physiol Biochem(3): 790-803.
Kimura, et al. (2013), Invest Ophthalmol Vis Sci(4): 2572-2760.
Lemp, et al. (2011), Am J Ophthalmol(5): 792-798.
Li, et al. (2024), Ocul Surf: 182-191.
Li, et al. (2021), Ocul Surf:163-171.
Liu, et al. (2022), Signal Transduct Target Ther(1): 3.
Luo, et al. (2007), Cornea(4): 452-460.
Ma, et al. (2021), Exp Biol Med (Maywood)(10): 1167-1176.
Maltseva, et al. (2013), PLoS One(8):e69642.
Pflugfelder, et al. (2005), Am J Pathol(1): 61-71.
Raz, et al. (2024), Invest Ophthalmol Vis Sci(6): 14.
Redfern, et al. (2013), Invest Ophthalmol Vis Sci(2): 1554-1563.
Robertson, et al. (2005), Invest Ophthalmol Vis Sci(2): 470-478.
Sharma, et al. (2023), Indian J Ophthalmol(4): 1154-1166.
Shen, et al. (2023), Exp Eye Res: 109418.
Song, et al. (2024), Cells(15).
Stuard Sambhariya, et al. (2023), J Biol Chem(11): 105239.
Van den Bossche, et al. (2012), Blood(7): 1623-1633.
Vereb, et al. (2016), Sci Rep: 26227.
Voss, et al. (2023), Int J Mol Sci(2).
Wang, et al. (2025), Int J Biol Macromol: 138267.
Woodward, et al. (2012), Exp Eye Res: 98-100.
Yurttaser Ocak, et al. (2020), Int Ophthalmol(5): 1135-1141.
Zhou, et al. (2022), JAMA Ophthalmol(4): 392-399.

### Example 5: L-Carnitine Mitigates Benzalkonium Chloride-Induced Ocular Surface Damage in a Murine Model of Dry Eye Disease

### Materials and Methods

### Marine Model of Dry Eye Disease

Male and female C57BL/6 mice (8-12 weeks, Jackson Laboratory, Bar Harbor, ME, USA) received topical administration of 5 μL of 0.2% BAC (B6295, Sigma-Aldrich, St. Louis, MO) in both eyes twice daily (9 am-9 pm) for 7 days. Each mouse was randomly assigned an experimental group including vehicle (water), BAC, BAC+L-carnitine, and L-carnitine alone. Mice in the treatment group received 0.2% BAC concurrently with 200 mM L-carnitine (840092, Sigma-Aldrich, St. Louis, MO) for 7 days. On the eighth day, ocular surface health was assessed, followed by euthanization and isolation of whole eyes or corneas. All animal studies were conducted in compliance with the Association for Research in Vision and Ophthalmology Statement (ARVO) Statement for the Use of Animals in Ophthalmic and Vision Research, approved by the Institutional Animal Care and Use Committee at Augusta University (protocol no. 2023-1107).

### Slit Lamp Microscopy and Fluorescein Staining

Mice were anesthetized using an intraperitoneal injection of ketamine and xylazine (100 mg/kg and 10 mg/kg respectively) then mounted on SL-DR slit-lamp (Topcon Medical Systems, NJ, USA) under white light at 40× magnification to examine corneal opacity. Scoring was done by multiplying the degree of opacity by its area, for a total maximum score of 16. Hence, the degree of opacity was scored as follows: 0, No corneal haze; 1, hazy but iris details visible; 2, pupillary margin visible but iris details not visible; 3, Pupillary margin not visible; and 4, cornea totally opaque. The area of opacity was scored as follows: 1, one quarter (or less) but not zero; 2, greater than one quarter, but less than one half, 3, greater than one half, but less than three quarters; and 4, greater than three quarters, up to the entire area.

To investigate corneal damage, 20 μL of sodium fluorescein dye from BioGlo strips (NC2128736, Thermo Fisher Scientific) was instilled onto each eye for 60 seconds. The dye was removed and then rinsed twice with DPBS and drained with Kimwipes (Ogawa, et al. (2013), Scientific Reports, 5:3:3419). The mice were mounted on the slit lamp and their corneas imaged using a cobalt blue filter. Images were taken at 40× magnification. To score, the corneal image was divided into five regions (nasal, temporal, superior, inferior, and central), and each scored using a previously validated grading system with modifications for mice; 0=no staining, 1=minimal staining, 2=mild/moderate staining, and 3=severe staining. Values in all regions were summed to a maximum score of 15.

### Measurement of Tear Volume

Tear production was measured by the phenol red thread test using cotton threads soaked in phenol red dye (Zone-Quick; Oasis, Glendora, CA, USA) for 5 minutes and air-dried. Mice were anesthetized and using curved forceps (Katena Products, Inc., Denville, NJ, USA), thread was gently placed in the lateral cantus of the conjunctival fornix of each eye for 20 sec. The length of wetting, evidenced by the red color change, was measured in millimeters (Lin, et al. (2011), Mol. Vis., 25:17:257-64).

### Hematoxylin and Eosin (H & E) Staining

Following euthanasia, whole eyes were enucleated and embedded in Tissue-Tek OCT compound (Sakura Finetek USA, Torrance, CA, USA), flash frozen in dry ice-ethanol and stored at −80 °C until further analysis. 7 m sections were stained with H & E for histological analysis. Slides were imaged at 2× and 20× magnification with the Keyence BZ-X710 microscope (Itasca, IL, USA). The thickness of the total cornea and the epithelial layer only was measured using the ImageJ (NIH, Bethesda, MD, USA).

Immunofluorescence Staining Corneal sections (7 μm) were fixed in 4% paraformaldehyde, permeabilized with 1% Triton X-100 in PBS for 5 minutes at room temperature, and blocked with 10% normal goat serum (NGS) for 1 hour. Incubation with primary antibodies against CPT1, CPT2, and CrAT was done at 4 °C overnight in a humidified chamber to prevent drying. Slides were incubated with Alexa Fluor 488 or 546 secondary antibodies (Invitrogen) for 1 hour at room temperature, mounted with DAPI-containing medium and fluorescent images captured at 20× magnification using a Keyence BZ-X710 microscope (Itasca, IL, USA). Mean fluorescence intensity was quantified with ImageJ (NIH, Bethesda, MD, USA).

### Western Blotting Analysis

Mice corneas were isolated, homogenized in cold RIPA buffer containing protease inhibitor and phosphatase inhibitor by sonication to extract proteins. Protein concentration was determined by BCA Assay. Samples were separated on 4-15% SDS-PAGE gels and transferred to nitrocellulose membranes using a Trans-Blot Turbo system (Bio-Rad). Membranes were blocked in 5% milk in TBS-T for 2 hours, incubated overnight at 4 °C with antibodies against E-cadherin (1:1,000; Cat #sc-376751, Santa Cruz), ALDH3A1 (1:1,000; Cat #sc-376751, Santa Cruz), and Keratocan (1:1,000; Cat #sc-376751, Santa Cruz) followed by HRP-conjugated secondary antibody (1:1,000; Cat #sc-376751, Santa Cruz) for 1 hour at room temperature. Blots were developed using ECL substrate and imaged with a ChemiDoc system (Bio-Rad). Band intensities were quantified using ImageJ and normalized to β-actin, with expression reported as fold change relative to the vehicle.

### In Situ TUNEL Assay

Terminal deoxynucleotidyl transferase dUTP nick end labeling (TUNEL) staining was performed using ApopTag Assay kit to assess apoptosis in corneal cryosections. Briefly, sections were fixed in 4% paraformaldehyde, permeabilized with 0.1% Triton X-100/0.1% sodium citrate solution for 2 minutes at 4 °C. Sections were rinsed in PBS equilibrated for 10 minutes at room temperature prior to labeling. The TUNEL reaction mixture, containing TdT enzyme and fluorescein-dUTP reaction buffer (1:9), was applied and incubated for 1 hour in a humidified chamber at 37 °C. Slides were then washed and incubated with anti-digoxigenin-FITC antibody for 30 minutes at 37 °C. Nuclei were counterstained with DAPI and images were acquired at 40× magnification using the Leica STELLARIS confocal microscope. TUNEL-positive cells were quantified by averaging the TUNEL-positive cells from four sections per eye per animal (Ferrington, et al. (2013), PloS One, 8(1):e54347).

### Statistical Analysis

Statistical analyses were performed with GraphPad Prism software 10.0 (GraphPad Software, Inc., San Diego, CA, USA) using One-way ANOVA with Tukey's multiple comparison's test. p-values <0.05 were considered statistically significant.

### Results

### L-Carnitine Ameliorates BAC-Induced Corneal Opacity in Male and Female Mice.

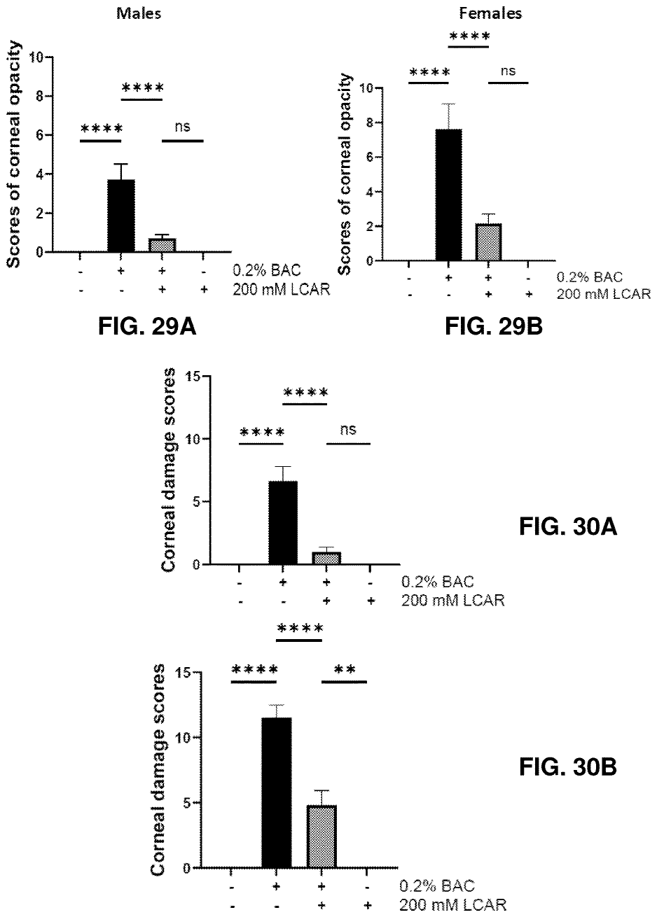

To characterize the sex-specific effects of BAC, the extent of corneal opacity was examined in male and female mice exposed to 0.2% BAC and the potential protective effects of L-carnitine (LCAR). Using the slit lamp, severe hazing of the cornea was observed in both male and female mice, with the females showing more corneal haze (data not shown). These changes were evaluated by scoring which revealed mean opacity scores of 4 and 7 in male and female DED mice respectively (FIGS. 29A and 29B). Treatment with LCAR significantly reduced the opacity scores to 1 in males and 2 in females (p<0.001), indicating protection against BAC's effects. Mice that received LCAR alone showed no hazing of the cornea.

### L-Carnitine Improves BAC-Associated Corneal Damage and Tear Insufficiency in Male and Female Mice

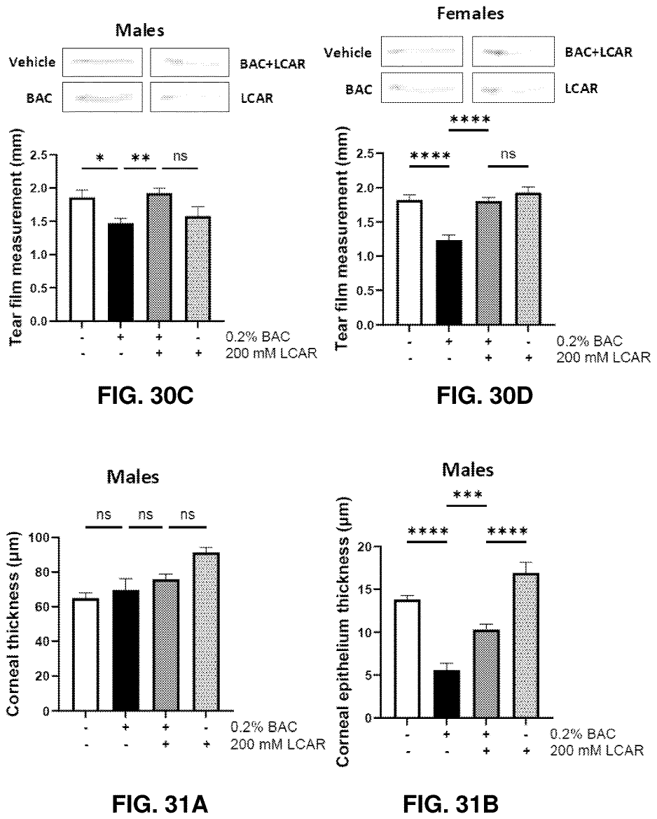

Using the fluorescein sodium dye under the slit lamp, the degree of corneal damage was examined in both male and female mice following BAC with or without LCAR treatments (data not shown). Intense dye staining was observed in both male and female BAC-only treated mice compared to the vehicle group, indicating severe epithelial damage. However, the females demonstrated higher degree of staining than the males. Scores of the corneal damage revealed similar observations where DED male mice recorded a score of 6 whilst females recorded 11 compared to the vehicle group (FIGS. 30A and 30B). Mice that received both BAC and LCAR recorded lower corneal damage scores, with the males recording 1 whilst females recorded 5, demonstrating LCAR's efficiency in mitigating the BAC-induced damage. Treatment with LCAR did not induce any corneal damage.

Inadequate tear volume is a central characteristic of DED, hence the effects of BAC and LCAR treatments on tear production were investigated. Using the phenol red thread method, significantly decreased tear production was observed in both male and female DED mice compared to vehicle controls, with females recording lower tear production than males (FIGS. 30C and 30D). Mice that received BAC and LCAR showed significantly increased restoration of tear levels relative to the BAC only groups. This highlights LCAR's protection against the BAC-induced tear insufficiency. LCAR alone did not alter tear levels in both mice sexes.

### Corneal Morphology is Preserved by L-Carnitine in Male and Female BAC-Treated Mice.

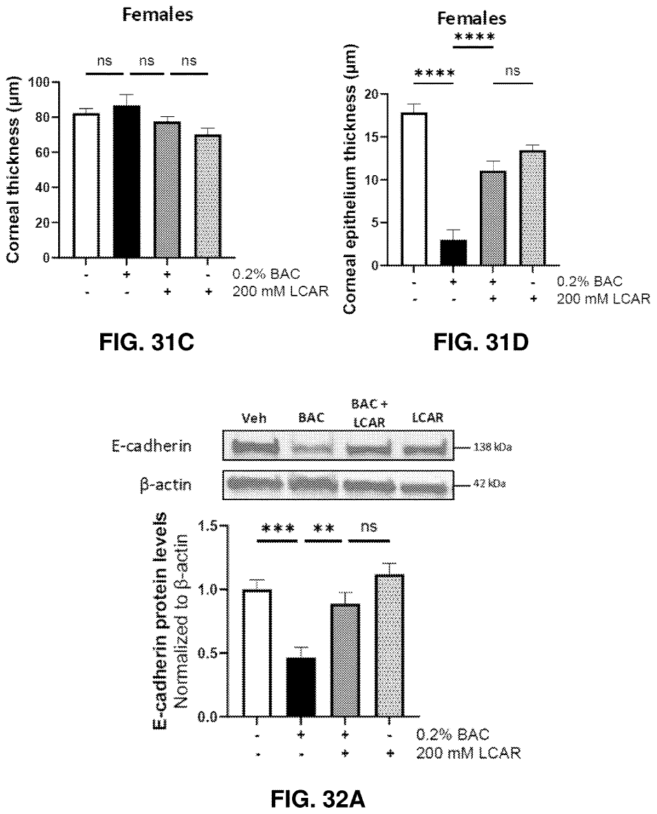

Histological examinations of the corneal morphology in both male and female mice after exposure to BAC treatment revealed significant alterations in the corneal layers including the epithelium and stroma (data not shown). Compared to the vehicle group, the BAC only group had prominent thinning of the epithelial layer with the female DED mice showing more significant thinning than males consistent with the measurements of the corneal epithelial layer (FIGS. 31B and 31F). Similarly, the BAC group demonstrated disorderly and misarranged stromal fibers with more nuclear staining than the controls suggesting immune cell infiltration and was more severe in females than males. Administration of BAC and LCAR showed significant alleviation of these corneal defects where LCAR significantly improved corneal epithelium thickness relative to the BAC only group. Measurement of total corneal thickness showed no significant changes across groups in both males and females (FIGS. 31A and 31E) which may be due to the cellular infiltration and edema making up for the loss of the epithelium in the BAC only group. Again, LCAR only treatment did not produce any corneal defects.

BAC-Impaired Corneal Epithelial and Stromal Layers are Protected with L-Carnitine Treatment.

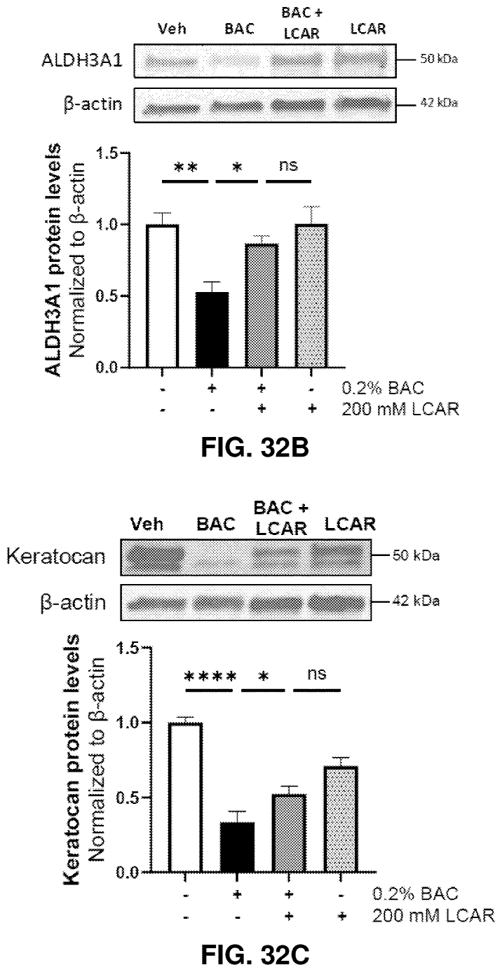

Following the above findings, the molecular changes that occur with BAC and LCAR treatments on markers of the corneal epithelium and stroma using combined corneal homogenates of male and female mice were investigated. Western blot analysis revealed that corneal epithelial integrity marker, E-cadherin, was significantly reduced in the BAC only group compared to the vehicle (FIG. 32A). LCAR treatment significantly restored E-cadherin levels relative to the BAC only group, emphasizing its ability to mitigate the BAC-induced impairment of corneal epithelial integrity.

Stromal markers ALDH3A1 and Keratocan were also assessed for the protein levels in corneas exposed to BAC with or without LCAR. ADH3A1 levels were significantly reduced in the BAC group relative to the vehicle group (FIG. 32B). Concurrent treatment with BAC and LCAR increased its levels relative to the BAC only group. Similarly, BAC induced the downregulation of Keratocan levels compared to the vehicle (FIG. 32C). However, corneas exposed to BAC and LCAR had higher Keratocan levels compared to BAC only group. In all, these findings suggest the dysregulation of the corneal stroma as was observed in our histological investigations and LCAR is effective in alleviating these BAC defects.

### L-Carnitine Restores BAC-Induced Dysregulation of Carnitine Metabolism Enzymes.

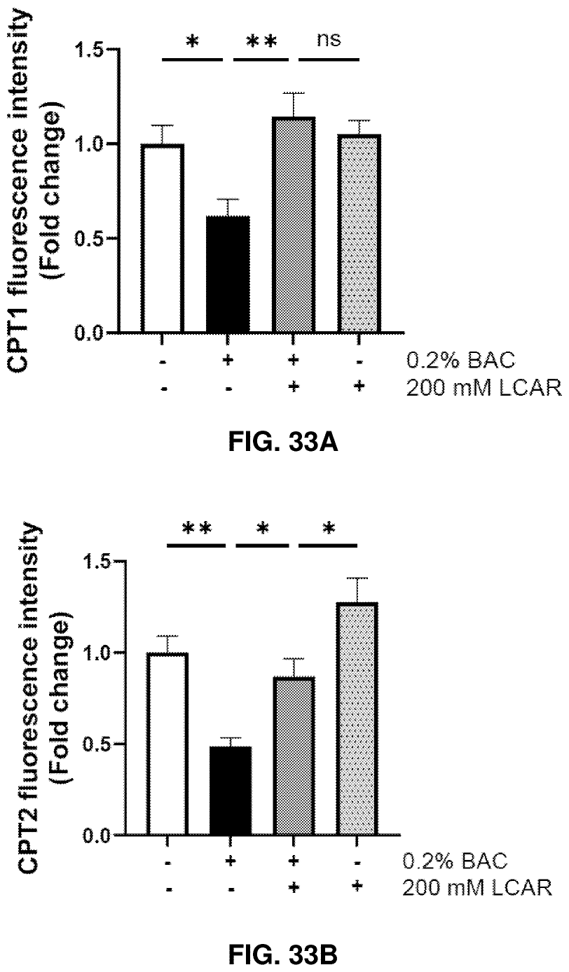

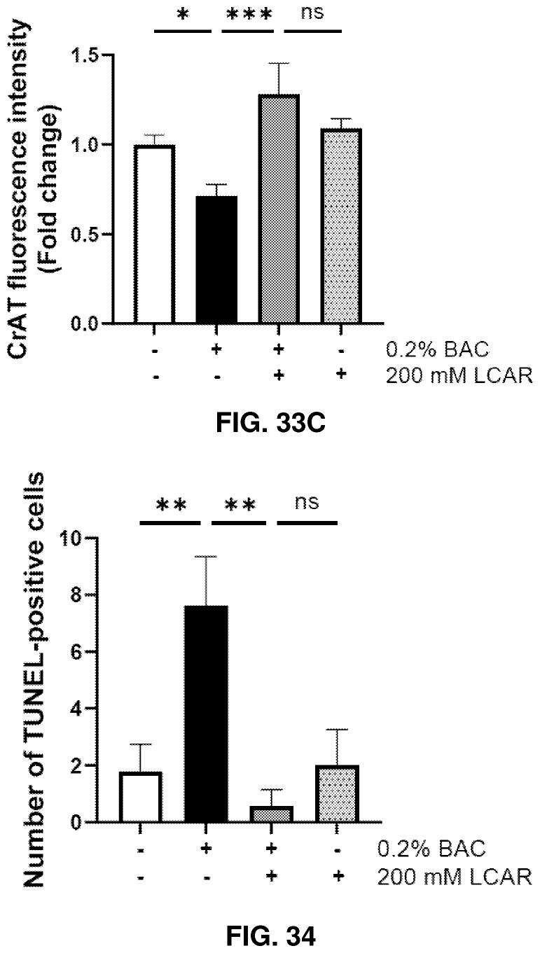

To understand the mechanisms by which LCAR may be mediating its protective effects the carnitine metabolism pathway was examined. It was evaluated whether L-carnitine, targeted the mitochondrial carnitine shuttle. Immunofluorescence staining of carnitine palmitoyltransferase (CPT1, CPT2) and carnitine acetyltransferase (CrAT) enzymes were performed, which together regulate the acyl-CoA and acetyl-CoA balance in the mitochondria. It was observed that CPT1, CPT2 and CrAT enzymes decreased in expression after BAC only treatments compared to the vehicle group (data not shown). Quantitative assessment showed significantly reduced fluorescence intensity in CPT1, CPT2, and CrAT. Administration of LCAR significantly improved the levels of CPT1, CPT2, and CrAT compared to the BAC only group (FIG. 33A, 33B, 33C). This demonstrates the protective effect of LCAR against the BAC-induced downregulation of carnitine enzymes.

BAC-Associated Apoptosis in Male and Female Corneas is Rescued with L-Carnitine Administration.

The protective effects of LCAR against BAC-induced cell death using the TUNEL assay in corneal sections was also investigated. The results showed that BAC caused apoptosis in the mice corneas compared to the vehicle (FIG. 34). Treatment with BAC and LCAR significantly mitigated the cellular apoptosis demonstrating LCAR's protection against BAC-induced damage (FIG. 34). Administration of LCAR alone showed no significant induction of apoptosis in the mice corneas (FIG. 34).

### Discussion

Benzalkonium chloride (BAC), a quaternary ammonium compound widely used as a preservative in more than 70% of topical ophthalmic drugs, has been shown to induce ocular surface toxicity with chronic exposure (Goldstein, et al. (2022), Eye (London), 36(2):361-368)). Clinical and experimental studies provide evidence that BAC can result in corneal epithelial damage, conjunctival inflammation, goblet cell loss, tear film instability, and increased ocular surface defects, all important hallmarks of DED (Liu, et al. (2015), Chin Med J, 128(18):2444-9; Muzyka-Wozniak, et al. (2023), Science Reports, 13(1):18625; Zhang, et al. (2020), Ocul Surf, 18(1):158-169; Datta, et al., (2017), Invest Ophthalmol Vis Sci., 58(4):2406-2412; Goldstein, et al. (2022), Eye (London), 36(2):361-368)). Mechanistically, BAC can disrupt epithelial cellular homeostasis, and promote mitochondrial dysfunction and apoptosis (Zheng, et al. (2025), Trans Vis Sci Technol., 14(11):16; Pauly, et al. (2011), Mol Vis. 17:745-55; Rogov, et al. (2020), Oxid Med Cell Longev., 10:2020:8956504).

L-carnitine is endogenously present in human tears and contributes to osmotic regulation at the ocular surface, with reduced levels reported in patients with dry eye disease (Pescosolido, et al. (2009), Optom Vis Sci., 86(2):E132-8). As demonstrated in Example 3 above, L-carnitine, an osmoprotectant, mitigates hyperosmolarity induced oxidative stress, lipotoxicity and mitochondrial dysfunction by preserving epithelial junctions, and promoting wound healing in human corneal epithelial cells.

Biological sex is increasingly recognized as an important determinant of ocular surface physiology and DED susceptibility. Epidemiological studies show that DED is more prevalent and severe in women, with higher symptom scores and a weaker correlation between symptoms and signs as compared to men (Vehof, et al. (2018), Ocul Surf 16(2): 242-248). These differences affect tear film stability, meibomian and lacrimal gland function, and lipid layer characteristics suggesting their modulation by sex steroid hormones that regulate tear production, lipid secretion, and inflammation (Noland, et al. (2021), Ocul Surf, 19:68-73; Kastelan, et al. (2025), Int J Mol Sci., 26(8):3833).

In the present study, a BAC-induced mouse model was used to investigate sex-specific differences in corneal pathology and the mechanisms by which L-carnitine restores tear production, prevents epithelial thinning and protects against BAC-induced corneal damage, thus highlighting its therapeutic potential for DED.

It was demonstrated that topical benzalkonium chloride (BAC) exposure causes characteristic features of dry eye disease (DED), including corneal damage, tear film dysfunction, and epithelial remodeling, with significantly more severe pathology in female mice compared to males. Importantly, it was shown that L-carnitine, a mitochondrial osmoprotectant, mitigates these BAC-induced changes by preserving epithelial integrity, tear volume, and expression of carnitine metabolism enzymes. These results provide mechanistic insight into BAC toxicity, therapeutic potential of L-carnitine, and highlight sex as an important biological variable in preclinical ocular surface models.

BAC is widely used as a preservative in nearly 70% of all multidose ophthalmic solutions due to its antimicrobial efficacy, yet it is also a well-characterized cytotoxic agent to ocular surface tissues (Goldstein, et al. (2022), Eye (London), 36(2):361-368). Its surfactant properties disrupt epithelial membranes, increase inflammatory mediator expression, and reduce mucin-producing goblet cells, leading to tear film instability and ocular surface staining characteristic of DED in both clinical and experimental contexts. Existing evidence indicates that chronic use of BAC-preserved glaucoma medications is associated with increased ocular surface staining, reduced tear break-up time, and worsened ocular surface disease symptoms (Liu, et al. 2015), Chin Med J, 128(18):2444-9). Preclinical work using murine and rabbit models indicate that topical BAC application produces punctate epithelial erosions, goblet cell loss, and increased barrier permeability indicative of DED-like pathology (Xiong, et al. (2008), Invest Ophthalmol Vis Sci., 49(5):1850-6; Zhang, et al. (2020), Ocul Surf, 18(1):158-169). In-vitro evidence indicates that BAC can inhibit mitochondrial respiratory function in corneal epithelial cells and triggers cytotoxicity at concentrations relevant to pharmaceutical use, suggesting that mitochondrial-related toxic mechanisms may underlie BAC-associated ocular surface injury (Datta, et al., (2017), Invest Ophthalmol Vis Sci., 58(4):2406-2412).

Moreover, BAC toxicity has been shown to be dose-dependent, with higher concentrations and repeated exposure producing more severe epithelial defects and inflammation, while even low concentrations can impair cell viability and colony formation in cultured ocular epithelial cells (Zhang, et al. (2020), Ocul Surf, 18(1):158-169). In the present model, chronic application of 0.2% BAC produced clinically and histologically detectable surface damage within one week, consistent with its use as an acute DED model in C57BL/6 mice. Together, these data support the validity of BAC-induced ocular surface injury as a robust model for studying the mechanisms and potential interventions for preservative-associated DED.

DED is fundamentally associated with tear film hyperosmolarity, oxidative stress, and inflammation. Hyperosmolar stress increases reactive oxygen species (ROS) in corneal epithelial cells, triggering inflammatory cascades and apoptosis that further destabilize the tear film and compromise barrier integrity (Hua, et al. (2015), Invest Ophthalmol Vis Sci., 56(9):5503-5511). L-carnitine is an endogenous osmolyte present in human tear fluid and has been implicated in tear osmolarity regulation, suggesting a physiological role in ocular surface homeostasis (Pescosolido, et al. (2009), Optom Vis Sci(2):E132-E138). L-carnitine supplementation can reduce ROS production and oxidative damage in human corneal epithelial cells exposed to hyperosmolar stress, preserving antioxidant enzyme activity and reducing mitochondrial DNA damage. These antioxidative and osmoprotective properties provide a credible mechanistic basis for its observed protective effects in the present BAC model.

The present findings extend these observations by showing that L-carnitine not only mitigates epithelial thinning and surface opacity but also prevents downregulation of key mitochondrial carnitine pathway enzymes (CPT1, CPT2, and CrAT). This implicates mitochondrial metabolic preservation as a component of L-carnitine's protective mechanism, suggesting that preserving mitochondrial function may represent a viable therapeutic strategy against preservative-induced ocular surface toxicity.

Sex differences in DED prevalence, symptoms, and underlying physiology are well established in clinical populations, with women disproportionately affected. It has been shown that women are more likely to report severe symptoms and exhibit distinct patterns of signs and symptoms compared to men, highlighting sex as a relevant factor in ocular surface disease (Vehof, et al. (2018), Ocul Surf 16(2): 242-248). Sex hormones, including androgens and estrogens, influence the lacrimal and meibomian glands, tear production, and ocular surface immune responses, thereby contributing to differential susceptibility and disease progression (Gorimanipalli, et al. (2023), Indian J Ophthalmol, 71(4):1276-1284; Noland, et al. (2021), Ocul Surf, 19:68-73). In particular, the lacrimal gland exhibits sexual dimorphism in structure and function, with evidence suggesting that androgen deficiency and age-related hormonal changes disproportionately impact female ocular surface health. (Kastelan, et al. (2025), Int J Mol Sci., 26(8):3833)

Animal studies further illustrate the importance of sex differences in ocular surface biology. For example, studies revealed that corneal innervation and nerve regeneration exhibit sex-linked variation in mice, with differences in nerve density and regenerative capacity which may influence injury responses (Pham, et al. (2018), FASEB J, 33(3):4598-4609). In experimental autoimmune dry eye models, female mice have been reported to develop more severe disease earlier than males, mirroring clinical sex disparities in human DED (Li, et al., (2022), Invest Ophthalmol Vis Sci., 63(6):18). Another study demonstrated sex-specific responses to ocular surface stressors where female mice exhibited greater corneal hypersensitivity and DED signs in response to lacrimal gland excision than males, suggesting intrinsic differences in ocular immune and pain signaling pathways. (Mecum, et al. (2020), Scientific Reports, 10, Article number: 17225)

The present observation that female mice exhibit greater BAC-induced corneal opacity and damage scores than males therefore support the inclusion of sex as a biological variable in preclinical models of ocular surface injury. Considering sex differences can reveal distinct pathways of susceptibility, injury repair, and response to therapeutics that would otherwise be obscured in studies conducted in a single sex.

The findings of the present study also demonstrate that L-carnitine reduces BAC-associated pathology suggest that osmoprotective and mitochondrial-targeted approaches can be leveraged to mitigate preservative toxicity in clinical settings. Given the increasing emphasis on developing preservative-free formulations and alternative preservatives with better safety profiles, utilizing metabolic-targeting strategies like L-carnitine supplementation could complement existing therapeutic interventions. Additionally, the sex differences observed in the present study emphasize the need for sex-balanced experimental design and suggest that sex-specific mechanisms should be considered when translating preclinical findings to clinical treatments for DED.

In conclusion, benzalkonium chloride induces hallmark features of dry eye disease, with more severe ocular surface damage observed in females than males, underscoring the importance of sex as a biological variable in preclinical models. Consistent with these effects, long-term use of BAC-preserved eye drops, particularly in glaucoma therapy, is associated with increased symptoms and signs of ocular surface disease, prompting efforts to reduce or eliminate preservative exposure in chronic treatment regimens. L-carnitine effectively mitigates BAC-induced corneal epithelial injury, tear film dysfunction, and metabolic disruption, supporting its role as a protective osmoprotective and mitochondrial-targeted intervention. Together, these findings provide mechanistic understanding into preservative-induced dry eye and support further evaluation of L-carnitine as a therapeutic strategy for managing ocular surface disease. L-carnitine can reduce corneal epithelial apoptosis, suppress inflammatory cytokine expression, and improves tear film homeostasis.

Kontoh-Twumasi R, Aliste A, Scheid A, Glass J, Sharma A, Sharma S. L-carnitine partially restores adherens junction integrity and promotes wound healing in human corneal epithelial cells exposed to hyperosmolar stress. Exp Eye Res. 2025 September; 258:110499. doi: 10.1016/j.exer.2025.110499. Epub 2025 Jun. 26. PMID: 40581139; and Richard Kontoh-Twumasi, Antonella Aliste, Alexander Scheid, Karina Lax, Joshua Glass, Ashok Sharma, Shruti Sharma; Altered Carnitine Homeostasis Modulates Hyperosmolarity-Induced Mitochondrial Dysfunction and Lipotoxicity in Human Corneal Epithelial Cells. Invest. Ophthalmol. Vis. Sci. 2026; 67(2):39. doi.org/10.1167/iovs.67.2.39 are specifically incorporated by reference in there entireties.

Those skilled in the art will recognize, or be able to ascertain, using no more than routine experimentation, many equivalents to the specific embodiments of the method and compositions described herein. Such equivalents are intended to be encompassed by the following claims.

All of the references cited herein are specifically incorporated by reference in their entireties.

We claim:

1. An ophthalmic composition comprising:
(i) one or more preservatives that causes hyperosmolarity in an amount effective to prevent or reduce microbial contamination, and
(ii) one or more osmoprotective agents in an amount effective to prevent or reduce preservative-induced hyperosmolarity and/or ocular surface damage.

2. The ophthalmic composition of claim 1, wherein
the one or more osmoprotective agents are selected from L-carnitine, L-carnitine derivative or analog thereof, wherein the L-carnitine derivative or analog is selected from the group consisting of acetylcarnitine (C2:0), propionylcarnitine (C3:0), butyrylcarnitine (C4:0), and valerylcarnitine (C5:0), and combinations thereof and/or
the one or more preservatives is selected from a quaternary ammonium optionally benzalkonium chloride (BAC), polyquaternium-1, polyquaternium-42, or cetrimide; an oxidizing agent optionally sodium perborate or stabilized oxochloro complex; an ionic buffer optionally borate, sorbital, propylene glycol, or zing; an amidine optionally chlorhexidine; a paraben optionally methylparaben or propylparaben; an alcohol optionally chlorobutanol or phenylethanol; a mercury-based chemical optionally thimerosal or phenylmercuric nitrate/acetate; and combinations thereof.

3. The ophthalmic composition of claim 1, wherein preservative is present in the composition in a concentration ranging from 0.001% wt/v to 0.02% wt/v.

4. The ophthalmic composition of claim 1, wherein the one or more osmoprotective agents are present in the composition in a concentration ranging from 0.1% wt/v to 5.0% wt/v.

5. The ophthalmic composition of claim 1, further comprising a pharmaceutically acceptable carrier suitable for ocular administration.

6. The ophthalmic composition of claim 5, wherein the pharmaceutically acceptable carrier comprises an aqueous buffer solution with a pH between 6.5 and 7.8.

7. The ophthalmic composition of claim 1, further comprising one or more additional agents selected from the group consisting of viscosity-enhancing agents; chelating agents; pH-adjusting agents; stabilizers; buffering agents, lubricants, tonicity adjusting agents, demulcents, and preservatives.

8. The ophthalmic composition of claim 1, further comprising one or more additional active agents selected from the group consisting of anti-glaucoma agents, anti-inflammatory agents, antihistamines or mast cell stabilizers, antibiotics or antiviral agents.

9. The ophthalmic composition of claim 1, wherein the ophthalmic composition is formulated as an aqueous solution, suspension, gel, ointment, or emulsion suitable for ocular administration.

10. The ophthalmic composition of claim 1, wherein the ophthalmic composition is incorporated into an ocular delivery system selected from the group consisting of ocular wound bandages, ocular inserts, ocular gel drops, contact lenses, contact lens solution, and periocular patches.

11. The ophthalmic composition of claim 1, wherein the composition is formulated as artificial tears, an anti-glaucoma medication, an ophthalmic lubricant, an anti-allergy eye drop, an anti-inflammatory eye drop, a post-operative ophthalmic treatment, an ocular wound healing aid, an antimicrobial ophthalmic preparation, a mydriatic eye drop, cycloplegic eye drops, a prophylactic or therapeutic product for treating or managing ocular surface or anterior segment conditions, or a combination thereof.

12. The ophthalmic composition of claim 1, having an osmolality ranging from 150 mOsm/kg to 500 mOsm/kg, optionally wherein the osmolality ranges from 280 mOsm/kg to 320 mOsm/kg.

13. A method of reducing or preventing or treating preservative-induced ocular damage, comprising administering to one or both eyes of a subject in need thereof, the ophthalmic composition of claim 1.

14. A method of reducing or preventing or treating preservative-induced hyperosmolarity in a subject in need thereof, comprising administering to one or both eyes of a subject in need thereof, the ophthalmic composition of claim 1.

15. The method of claim 13, wherein the composition is administered to a subject undergoing topical ophthalmic therapy requiring medication.

16. The method of claim 13, wherein the ophthalmic composition is administered to treat, prevent, or as part of a therapeutic regimen for a condition associated with ocular surface hyperosmolarity, wherein the condition is selected from the group consisting of dry eye disease, keratoconjunctivitis sicca, meibomian gland dysfunction, allergic conjunctivitis, blepharitis, corneal epithelial defects, superficial punctate keratitis, conjunctival inflammation, corneal abrasion, ocular graft-versus-host disease, post-surgical ocular surface inflammation, contact lens-related discomfort, post-operative ocular irritation, environmentally induced dry eye, or any combination thereof.

17. The method of claim 13, wherein the composition reduces one or more markers of ocular surface toxicity selected from the group consisting of epithelial cell apoptosis, adherens junction disruption, tear film instability, corneal staining, and inflammation, in the subject, compared to the same subject prior to administration or a subject not administered the composition or the subject administered the same composition without the osmoprotective agent.

18. The method of claim 13, wherein the subject is selected from the group consisting of patients with ocular surface disorders, individuals undergoing long-term topical ophthalmic therapy, post-operative patients recovering from refractive or cataract surgery, elderly individuals with age-related tear film instability, contact lens wearers, and patients with autoimmune conditions affecting the eye (such as Sjögren's syndrome or ocular graft-versus-host disease), or a combination thereof.

19. The method of claim 13, wherein the ophthalmic composition is administered topically to the ocular surface one to six times daily.

20. The method of claim 13, wherein the ophthalmic composition is administered as part of an acute or chronic treatment regimen tailored to the severity of the ocular surface condition.

21. The method of claim 13, wherein administering the ophthalmic composition to the subject increases expression of one or more enzymes selected from CPT1, CPT2, and CrAT in corneal epithelial cells of the subject, compared to the same subject prior to administration of the ophthalmic composition or a different subject not administered the ophthalmic composition.

22. The method of claim 13, wherein administering the ophthalmic composition to the subject increases transepithelial electrical resistance (TER) in corneal epithelial cells of the subject, compared to the same subject prior to administration of the ophthalmic composition or a different subject not administered the ophthalmic composition.


SEQUENCE LISTING (18 sequences)

SEQ ID NO: 1 - DNA, 20 bp, synthetic construct
  source 1..20: mol_type = other DNA
cttgccctgagacggggatt

SEQ ID NO: 2 - DNA, 19 bp, synthetic construct
  source 1..19: mol_type = other DNA
tccagcccagcacatgaac

SEQ ID NO: 3 - DNA, 20 bp, synthetic construct
  source 1..20: mol_type = other DNA
gccaccatgaaaaccctcac

SEQ ID NO: 4 - DNA, 15 bp, synthetic construct
  source 1..15: mol_type = other DNA
gccctggccagcact

SEQ ID NO: 5 - DNA, 19 bp, synthetic construct
  source 1..19: mol_type = other DNA
gaccgtggtgaagcctctg

SEQ ID NO: 6 - DNA, 20 bp, synthetic construct
  source 1..20: mol_type = other DNA
accactcagacagccagttc

SEQ ID NO: 7 - DNA, 20 bp, synthetic construct
  source 1..20: mol_type = other DNA
aatgaaggggtcattgatgg

SEQ ID NO: 8 - DNA, 20 bp, synthetic construct
  source 1..20: mol_type = other DNA
aaggtgaaggtcggagtcaa

SEQ ID NO: 9 - DNA, 20 bp, synthetic construct
  source 1..20: mol_type = other DNA
gctggaccgagagagtttcc

SEQ ID NO: 10 - DNA, 20 bp, synthetic construct
  source 1..20: mol_type = other DNA
ggtgtatacagcctcccacg

SEQ ID NO: 11 - DNA, 20 bp, synthetic construct
  source 1..20: mol_type = other DNA
attgtccacgctggattttc

SEQ ID NO: 12 - DNA, 17 bp, synthetic construct
  source 1..17: mol_type = other DNA
tcgaggacggtcggact

SEQ ID NO: 13 - DNA, 22 bp, synthetic construct
  source 1..22: mol_type = other DNA
gctcacagtgtaataggggaga

SEQ ID NO: 14 - DNA, 21 bp, synthetic construct
  source 1..21: mol_type = other DNA
tggacagcagtcattttgcag

SEQ ID NO: 15 - DNA, 20 bp, synthetic construct
  source 1..20: mol_type = other DNA
agagcgttaccaagaggcag

SEQ ID NO: 16 - DNA, 21 bp, synthetic construct
  source 1..21: mol_type = other DNA
ggagaaccactcatctttgcc

SEQ ID NO: 17 - DNA, 20 bp, synthetic construct
  source 1..20: mol_type = other DNA
aatgaaggggtcattgatgg

SEQ ID NO: 18 - DNA, 20 bp, synthetic construct
  source 1..20: mol_type = other DNA
aaggtgaaggtcggagtcaa